(19) United States
(12) Patent Application Publication
(10) Pub. No.: US 2016/0339026 A1
(43) Pub. Date: Nov. 24, 2016

(54) TOPICAL COMPOSITIONS COMPRISING MODULATORS OF TRPM8

(71) Applicant: Senomyx, Inc., San Diego, CA (US)

(72) Inventors: Alain Noncovich, San Diego, CA (US); Jane Ung, San Diego, CA (US); Timothy Davis, Santee, CA (US); Joseph R. Fotsing, San Diego, CA (US); Rachel D.A. Kimmich, San Diego, CA (US); Chad Priest, Leucadia, CA (US); Donald S. Karanewsky, Escondido, CA (US); Lyudmyla Chumakova, San Diego, CA (US); Andrew Patron, San Marcos, CA (US); Paul Brust, San Diego, CA (US); Guy Servant, San Diego, CA (US)

(21) Appl. No.: 14/874,258

(22) Filed: Oct. 2, 2015

(51) Int. Cl. A61K 31/515; A61K 31/4155; A61K 31/4152; A61K 31/4406; A61K 31/513; A61K 31/4166; A61K 31/165; A61K 9/00; A61K 31/4439

(52) U.S. Cl. CPC A61K 31/515; A61K 9/0014; A61K 31/4155; A61K 31/4152; A61K 31/4439; A61K 31/513; A61K 31/4166; A61K 31/165; A61K 31/4406

(57) ABSTRACT

The present disclosure relates to topical compositions comprising one or more compounds which are cooling agents.

### BACKGROUND

1. Field of the Invention

The present disclosure relates to the field of topical compositions. More specifically, the present disclosure relates to topical compositions comprising compounds useful as cooling agents.

2. Background Description

Several formulations suitable for topical application contain agents that provide a cooling sensation. Among these cooling agents are compounds that are modulators of the Melastatin Transient Receptor Potential Channel 8 (TRPM8). TRPM8 is a channel involved in the chemesthetic sensation, such as cool to cold temperatures as well as the sensation of known cooling agents, such as Menthol and Icilin. However, many of the currently known TRPM8 modulators have deficiencies with regard to strength and or duration of effect, skin and/or mucosa irritation, odor, taste, solubility, and/or toxicity. Thus, there is a need for topical formulations having improved cooling agents.

### SUMMARY

In some embodiments of the present disclosure a topical composition is provided comprising a carrier and a compound of Formula (I):

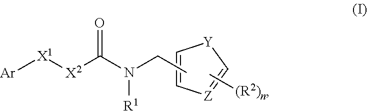

or a salt or solvate thereof;
wherein
Ar is optionally substituted aryl, optionally substituted carbocyclyl, or
optionally substituted heteroaryl;
Y is oxygen or sulfur;
Z is nitrogen or CR;
R is hydrogen or lower alkyl;
X¹-X² is O—CR^{2a}R^{2b}, CHR³—CHR⁴, or CR⁵═CR⁶;
R^{2a}, R^{2b}, R³, R⁴, R⁵, and R⁶ are independently hydrogen or lower alkyl;
R¹ is an optionally substituted five-membered heteroaryl;
n is 0, 1, 2, or 3; and
each R² is independently optionally substituted alkyl, optionally substituted heteroalkyl, optionally substituted alkenyl, alkoxy, hydroxyl, amino, N-alkyl amino, N-dialkyl amino, halo, nitro, cyano, acyl, carboxyl, carboxyl ester, or amide,
wherein each optional substituent is selected from the group consisting of alkyl, heteroalkyl, alkenyl, alkoxy, hydroxyl, amino, N-alkyl amino, N-dialkyl amino, halo, nitro, cyano, acyl, carboxyl, carboxyl ester, or amide; or two substituents, together with the atoms to which they are attached, form a carbocyclyl optionally substituted with alkyl or alkoxy; or two substituents, together with the atoms to which they are attached, form a heterocyclyl containing one or more heteroatom(s) selected from nitrogen, oxygen, and sulfur.

In some embodiments a topical composition is provided comprising a carrier and a compound of Formula (IIa) or (IIb):

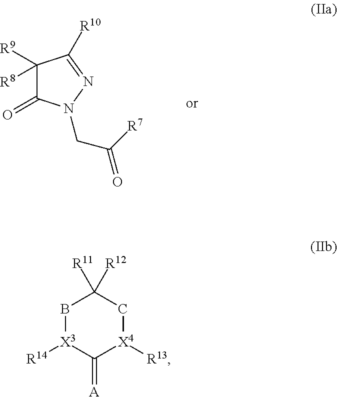

or a salt or solvate thereof;
wherein
   R⁷ is optionally substituted aryl, optionally substituted carbocyclyl, optionally substituted heteroaryl, or optionally substituted heterocyclyl;
   R⁸ and R⁹ are independently selected from the group consisting of optionally substituted alkyl, optionally substituted alkenyl, optionally substituted aryl, optionally substituted carbocyclyl, optionally substituted heteroaryl, and optionally substituted heterocyclyl;
   R¹⁰ is optionally substituted alkyl, optionally substituted alkenyl, optionally substituted aryl, optionally substituted carbocyclyl, optionally substituted heteroaryl, or optionally substituted heterocyclyl;
   either R¹¹ or R¹² is optionally substituted C₁-C₃ alkyl; and the remaining R¹¹ or R¹² is selected from the group consisting of optionally substituted alkyl, optionally substituted alkenyl, optionally substituted alkynyl, optionally substituted alkoxy, optionally substituted alkylaryl, optionally substituted alkoxyaryl, optionally substituted aryl, optionally substituted aryloxy, optionally substituted heteroalkyl, optionally substituted carbocyclyl, or optionally substituted heterocylcyl; or alternatively, R¹¹ and R¹², taken together with the atoms to which they are attached, form an optionally substituted carbocyclyl;
   X³ and X⁴ are independently CH or N; provided that X³ and X⁴ are not both CH;
   R¹³ and R¹⁴ are independently selected from the group consisting of hydrogen, halogen, hydroxyl, cyano, carboxy, optionally substituted C₁-C₈ alkyl, optionally substituted alkenyl, optionally substituted alkynyl, optionally substituted heteroalkyl, optionally substituted alkylaryl, optionally substituted alkoxyaryl, optionally substituted aryl, optionally substituted aryloxy, optionally substituted heteroaryl, optionally substituted heteroaryloxy, optionally substituted carbocyclyl, or optionally substituted heterocyclyl, -alkylene-carbonyl-aryl, -alkylene-carbonyl-heteroaryl, -alkylene-carbonyl-(substituted aryl), -alkylene-carbonyl-(substituted heteroaryl), -alkylene-carbonyl-O-aryl, -alkylene-carbonyl-O-(substituted aryl), -alkylene-carbonyl-NR¹⁵-aryl, -alkylene-carbonyl-NR¹⁵-(substituted aryl), -alkylene-carbonyl-O-heteroaryl, -alkylene-carbonyl-O-(substituted heteroaryl), -alkylene-carbonyl-NR¹⁵-heteroaryl, -alkylene-carbonyl-NR¹⁵-(substituted heteroaryl), OR¹⁵, and NR¹⁵R¹⁶; or alternatively, X³ and R¹⁴, or X⁴ and R¹³, taken together, is independently O or S;
   A is O, S, or NR¹¹;

B and C are independently CH₂, C═O, or a covalent bond; provided that B and C are not both covalent bonds; and

R¹⁵ and R¹⁶ are independently selected from the group consisting of hydrogen, optionally substituted alkyl, optionally substituted aryl, optionally substituted arylalkyl, optionally substituted heteroalkyl, optionally substituted heteroaryl, optionally substituted heteroarylalkyl, optionally substituted acylamido, and optionally substituted diacylamido; or alternatively, R⁹ and R¹⁰, together with the atoms to which they are bonded, form an optionally substituted cycloheteroalkyl.

In some embodiments, a topical composition is provided comprising a carrier and a compound of Formula (III):

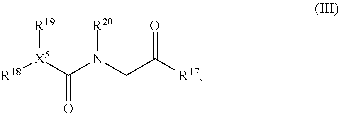

or a salt or solvate thereof;

wherein

X⁵ is CR²¹ or N;

R¹⁷ is optionally substituted aryl, optionally substituted carbocyclyl, optionally substituted heteroaryl, or optionally substituted heterocyclyl;

R¹⁸ and R¹⁹ are the same or different and are independently selected form the group consisting of hydrogen, optionally substituted alkyl, optionally substituted alkenyl, optionally substituted aryl, optionally substituted carbocyclyl, optionally substituted heteroaryl, and optionally substituted heterocyclyl;

R²⁰ is hydrogen, hydroxyl, alkoxy, optionally substituted alkyl, optionally substituted alkenyl, optionally substituted aryl, optionally substituted carbocyclyl, optionally substituted heteroaryl, or optionally substituted heterocyclyl; and

R²¹ is hydrogen, optionally substituted alkyl, optionally substituted alkenyl, optionally substituted aryl, optionally substituted carbocyclyl, optionally substituted heteroaryl, or optionally substituted heterocyclyl; or alternatively, R¹⁹ and R²¹, or R¹⁹ and R²⁰, taken together with the atoms to which they are attached, form an optionally substituted carbocyclyl or optionally substituted heterocyclyl.

### DETAILED DESCRIPTION

### Definitions

Unless defined otherwise, all technical and scientific terms used herein have the same meaning as is commonly understood by one of ordinary skill in the art to which this disclosure belongs. All patents, applications, published applications, and other publications are incorporated by reference in their entirety. In the event that there is a plurality of definitions for a term herein, those in this section prevail unless stated otherwise.

“Solvate” refers to the compound formed by the interaction of a solvent and a compound described herein or salt thereof. Suitable solvates are physiologically acceptable solvates including hydrates.

As used herein, “Cₐ to C_{b}” or “C_{a-b}” in which “a” and “b” are integers refer to the number of carbon atoms in the specified group. That is, the group can contain from “a” to “b”, inclusive, carbon atoms. Thus, for example, a “C₁ to C₄ alkyl” or “C₁₋₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—.

The term “halogen” or “halo,” as used herein, means any one of the radio-stable atoms of column 7 of the Periodic Table of the Elements, e.g., fluorine, chlorine, bromine, or iodine, with fluorine and chlorine being preferred.

As used herein, “alkyl” refers to a straight or branched hydrocarbon chain that is fully saturated (i.e., contains no double or triple bonds). The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 9 carbon atoms. The alkyl group could also be a lower alkyl having 1 to 4 carbon atoms. The alkyl group may be designated as “C₁₋₄ alkyl” or similar designations. By way of example only, “C₁₋₄ alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, and the like.

As used herein, “substituted alkyl” refers to an alkyl group substituted with one or more substituents independently selected from C₁-C₆ alkenyl, C₁-C₆ alkynyl, C₁-C₆ heteroalkyl, C₃-C₇ carbocyclyl (optionally substituted with halo, C₁-C₆ alkyl, C₁-C₆ alkoxy, C₁-C₆ haloalkyl, and C₁-C₆ haloalkoxy), 3-10 membered heterocyclyl (optionally substituted with halo, C₁-C₆ alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆ haloalkyl, and C₁-C₆ haloalkoxy), 5-10 membered heteroaryl (optionally substituted with halo, C₁-C₆ alkyl, C₁-C₆ alkoxy, C₁-C₆ haloalkyl, and C₁-C₆ haloalkoxy), halo, cyano, hydroxy, C₁-C₆ alkoxy, aryloxy (optionally substituted with halo, C₁-C₆ alkyl, C₁-C₆ alkoxy, C₁-C₆ haloalkyl, and C₁-C₆ haloalkoxy), C₃-C₇ carbocyclyloxy (optionally substituted with halo, C₁-C₆ alkyl, C₁-C₆ alkoxy, C₁-C₆ haloalkyl, and C₁-C₆ haloalkoxy), 3-10 membered heterocyclyl-oxy (optionally substituted with halo, C₁-C₆ alkyl, C₁-C₆ alkoxy, C₁-C₆ haloalkyl, and C₁-C₆ haloalkoxy), 5-10 membered heteroaryl-oxy (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkoxy (optionally substituted with halo, C₁-C₆ alkyl, C₁-C₆ alkoxy, C₁-C₆ haloalkyl, and C₁-C₆ haloalkoxy), 3-10 membered heterocyclyl-C₁-C₆-alkoxy (optionally substituted with halo, C₁-C₆ alkyl, C₁-C₆ alkoxy, C₁-C₆ haloalkyl, and C₁-C₆ haloalkoxy), aryl(C₁-C₆)alkoxy (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkoxy (optionally substituted with halo, C₁-C₆ alkyl, C₁-C₆ alkoxy, C₁-C₆ haloalkyl, and C₁-C₆ haloalkoxy), sulfhydryl (mercapto), halo(C₁-C₆)alkyl (e.g., —CF₃), halo(C₁-C₆)alkoxy (e.g., —OCF₃), C₁-C₆ alkylthio, arylthio (optionally substituted with halo, C₁-C₆ alkyl, C₁-C₆ alkoxy, C₁-C₆ haloalkyl, and C₁-C₆ haloalkoxy), C₃-C₇ carbocyclylthio (optionally substituted with halo, C₁-C₆ alkyl, C₁-C₆ alkoxy, C₁-C₆ haloalkyl, and C₁-C₆ haloalkoxy), 3-10 membered heterocyclyl-thio (optionally substituted with halo, C₁-C₆ alkyl, C₁-C₆ alkoxy, C₁-C₆ haloalkyl, and C₁-C₆ haloalkoxy), 5-10 membered heteroaryl-thio (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkylthio (optionally substituted with halo, C₁-C₆ alkyl, C₁-C₆ alkoxy, C₁-C₆ haloalkyl, and C₁-C₆ haloalkoxy), 3-10 membered heterocyclyl-C₁-C₆-alkylthio (optionally substituted with halo, C₁-C₆ alkyl, C₁-C₆ alkoxy, C₁-C₆ haloalkyl, and C₁-C₆ haloalkoxy), aryl(C₁-C₆)alkylthio (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkylthio (optionally substituted with halo, C₁-C₆ alkyl, C₁-C₆ alkoxy, C₁-C₆ haloalkyl, and C₁-C₆ haloalkoxy), amino, nitro, O-carbamyl, N-carbamyl, O-thiocarbamyl, N-thiocarbamyl, C-amido, N-amido, S-sulfonamido, N-sulfonamido, C-carboxy, O-carboxy, acyl, cyanato, isocyanato, thiocyanato, isothiocyanato, sulfinyl, sulfonyl, and oxo (═O).

As used herein, “alkoxy” refers to the formula —OR wherein R is an alkyl as is defined above, such as “C₁₋₉ alkoxy”, including but not limited to methoxy, ethoxy, n-propoxy, 1-methylethoxy (isopropoxy), n-butoxy, iso-butoxy, sec-butoxy, and tert-butoxy, and the like.

As used herein, “alkylthio” refers to the formula —SR wherein R is an alkyl as is defined above, such as “C₁₋₉ alkylthio” and the like, including but not limited to methylmercapto, ethylmercapto, n-propylmercapto, 1-methylethylmercapto (isopropylmercapto), n-butylmercapto, iso-butylmercapto, sec-butylmercapto, tert-butylmercapto, and the like.

As used herein, “alkenyl” refers to a straight or branched hydrocarbon chain containing one or more double bonds. The alkenyl group may have 2 to 20 carbon atoms, although the present definition also covers the occurrence of the term “alkenyl” where no numerical range is designated. The alkenyl group may also be a medium size alkenyl having 2 to 9 carbon atoms. The alkenyl group could also be a lower alkenyl having 2 to 4 carbon atoms. The alkenyl group may be designated as “C₂₋₄ alkenyl” or similar designations. By way of example only, “C₂₋₄ alkenyl” indicates that there are two to four carbon atoms in the alkenyl chain, i.e., the alkenyl chain is selected from the group consisting of ethenyl, propen-1-yl, propen-2-yl, propen-3-yl, buten-1-yl, buten-2-yl, buten-3-yl, buten-4-yl, 1-methyl-propen-1-yl, 2-methyl-propen-1-yl, 1-ethyl-ethen-1-yl, 2-methyl-propen-3-yl, buta-1,3-dienyl, buta-1,2,-dienyl, and buta-1,2-dien-4-yl. Typical alkenyl groups include, but are in no way limited to, ethenyl, propenyl, butenyl, pentenyl, and hexenyl, and the like.

As used herein, “alkynyl” refers to a straight or branched hydrocarbon chain containing one or more triple bonds. The alkynyl group may have 2 to 20 carbon atoms, although the present definition also covers the occurrence of the term “alkynyl” where no numerical range is designated. The alkynyl group may also be a medium size alkynyl having 2 to 9 carbon atoms. The alkynyl group could also be a lower alkynyl having 2 to 4 carbon atoms. The alkynyl group may be designated as “C₂₋₄ alkynyl” or similar designations. By way of example only, “C₂₋₄ alkynyl” indicates that there are two to four carbon atoms in the alkynyl chain, i.e., the alkynyl chain is selected from the group consisting of ethynyl, propyn-1-yl, propyn-2-yl, butyn-1-yl, butyn-3-yl, butyn-4-yl, and 2-butynyl. Typical alkynyl groups include, but are in no way limited to, ethynyl, propynyl, butynyl, pentynyl, and hexynyl, and the like.

As used herein, “heteroalkyl” refers to a straight or branched hydrocarbon chain containing one or more heteroatoms, that is, an element other than carbon, including but not limited to, nitrogen, oxygen and sulfur, in the chain backbone. The heteroalkyl group may have 1 to 20 carbon atom, although the present definition also covers the occurrence of the term “heteroalkyl” where no numerical range is designated. The heteroalkyl group may also be a medium size heteroalkyl having 1 to 9 carbon atoms. The heteroalkyl group could also be a lower heteroalkyl having 1 to 4 carbon atoms. The heteroalkyl group may be designated as “C₁₋₄ heteroalkyl” or similar designations. The heteroalkyl group may contain one or more heteroatoms. By way of example only, “C₁₋₄ heteroalkyl” indicates that there are one to four carbon atoms in the heteroalkyl chain and additionally one or more heteroatoms in the backbone of the chain.

As used herein, “alkylene” means a branched, or straight chain fully saturated di-radical chemical group containing only carbon and hydrogen that is attached to the rest of the molecule via two points of attachment (i.e., an alkanediyl). The alkylene group may have 1 to 20 carbon atoms, although the present definition also covers the occurrence of the term alkylene where no numerical range is designated. The alkylene group may also be a medium size alkylene having 1 to 9 carbon atoms. The alkylene group could also be a lower alkylene having 1 to 4 carbon atoms. The alkylene group may be designated as “C₁₋₄ alkylene” or similar designations. By way of example only, “C₁₋₄ alkylene” indicates that there are one to four carbon atoms in the alkylene chain, i.e., the alkylene chain is selected from the group consisting of methylene, ethylene, ethan-1,1-diyl, propylene, propan-1,1-diyl, propan-2,2-diyl, 1-methyl-ethylene, butylene, butan-1,1-diyl, butan-2,2-diyl, 2-methyl-propan-1,1-diyl, 1-methyl-propylene, 2-methyl-propylene, 1,1-dimethyl-ethylene, 1,2-dimethyl-ethylene, and 1-ethyl-ethylene.

As used herein, “alkenylene” means a straight or branched chain di-radical chemical group containing only carbon and hydrogen and containing at least one carbon-carbon double bond that is attached to the rest of the molecule via two points of attachment. The alkenylene group may have 2 to 20 carbon atoms, although the present definition also covers the occurrence of the term alkenylene where no numerical range is designated. The alkenylene group may also be a medium size alkenylene having 2 to 9 carbon atoms. The alkenylene group could also be a lower alkenylene having 2 to 4 carbon atoms. The alkenylene group may be designated as “C₂₋₄ alkenylene” or similar designations. By way of example only, “C₂₋₄ alkenylene” indicates that there are two to four carbon atoms in the alkenylene chain, i.e., the alkenylene chain is selected from the group consisting of ethenylene, ethen-1,1-diyl, propenylene, propen-1,1-diyl, prop-2-en-1,1-diyl, 1-methyl-ethenylene, but-1-enylene, but-2-enylene, but-1,3-dienylene, buten-1,1-diyl, but-1,3-dien-1,1-diyl, but-2-en-1,1-diyl, but-3-en-1,1-diyl, 1-methyl-prop-2-en-1,1-diyl, 2-methyl-prop-2-en-1,1-diyl, 1-ethyl-ethenylene, 1,2-dimethyl-ethenylene, 1-methyl-propenylene, 2-methyl-propenylene, 3-methyl-prop enylene, 2-methyl-propen-1,1-diyl, and 2,2-dimethyl-ethen-1,1-diyl.

The term “aromatic” refers to a ring or ring system having a conjugated pi electron system and includes both carbocyclic aromatic (e.g., phenyl) and heterocyclic aromatic groups (e.g., pyridine). The term includes monocyclic or fused-ring polycyclic (i.e., rings which share adjacent pairs of atoms) groups provided that the entire ring system is aromatic.

As used herein, “aryl” refers to an aromatic ring or ring system (i.e., two or more fused rings that share two adjacent carbon atoms) containing only carbon in the ring backbone. When the aryl is a ring system, every ring in the system is aromatic. The aryl group may have 6 to 18 carbon atoms, although the present definition also covers the occurrence of the term “aryl” where no numerical range is designated. In some embodiments, the aryl group has 6 to 10 carbon atoms. The aryl group may be designated as “C₆₋₁₀ aryl,” “C₆ or C₁₀ aryl,” or similar designations. Examples of aryl groups include, but are not limited to, phenyl, naphthyl, azulenyl, and anthracenyl.

As used herein, “aryloxy” and “arylthio” refers to RO— and RS—, in which R is an aryl as is defined above, such as “C₆₋₁₀ aryloxy” or “C₆₋₁₀ arylthio” and the like, including but not limited to phenyloxy.

An “aralkyl” or “arylalkyl” is an aryl group connected, as a substituent, via an alkylene group, such as “C₇₋₁₄ aralkyl” and the like, including but not limited to benzyl, 2-phenylethyl, 3-phenylpropyl, and naphthylalkyl. In some cases, the alkylene group is a lower alkylene group (i.e., a C₁₋₄ alkylene group).

As used herein, “heteroaryl” refers to an aromatic ring or ring system (i.e., two or more fused rings that share two adjacent atoms) that contain(s) one or more heteroatoms, that is, an element other than carbon, including but not limited to, nitrogen, oxygen and sulfur, in the ring backbone. When the heteroaryl is a ring system, every ring in the system is aromatic. The heteroaryl group may have 5-18 ring members (i.e., the number of atoms making up the ring backbone, including carbon atoms and heteroatoms), although the present definition also covers the occurrence of the term “heteroaryl” where no numerical range is designated. In some embodiments, the heteroaryl group has 5 to 10 ring members or 5 to 7 ring members. The heteroaryl group may be designated as “5-7 membered heteroaryl,” “5-10 membered heteroaryl,” or similar designations. Examples of heteroaryl rings include, but are not limited to, furyl, thienyl, phthalazinyl, pyrrolyl, oxazolyl, thiazolyl, imidazolyl, pyrazolyl, isoxazolyl, isothiazolyl, triazolyl, thiadiazolyl, pyridinyl, pyridazinyl, pyrimidinyl, pyrazinyl, triazinyl, quinolinyl, isoquinlinyl, benzimidazolyl, benzoxazolyl, benzothiazolyl, indolyl, isoindolyl, and benzothienyl.

A “heteroaralkyl” or “heteroarylalkyl” is heteroaryl group connected, as a substituent, via an alkylene group. Examples include but are not limited to 2-thienylmethyl, 3-thienylmethyl, furylmethyl, thienylethyl, pyrrolylalkyl, pyridylalkyl, isoxazollylalkyl, and imidazolylalkyl. In some cases, the alkylene group is a lower alkylene group (i.e., a C₁₋₄ alkylene group).

As used herein, “carbocyclyl” means a non-aromatic cyclic ring or ring system containing only carbon atoms in the ring system backbone. When the carbocyclyl is a ring system, two or more rings may be joined together in a fused, bridged or spiro-connected fashion. Carbocyclyls may have any degree of saturation provided that at least one ring in a ring system is not aromatic. Thus, carbocyclyls include cycloalkyls, cycloalkenyls, and cycloalkynyls. The carbocyclyl group may have 3 to 20 carbon atoms, although the present definition also covers the occurrence of the term “carbocyclyl” where no numerical range is designated. The carbocyclyl group may also be a medium size carbocyclyl having 3 to 10 carbon atoms. The carbocyclyl group could also be a carbocyclyl having 3 to 6 carbon atoms. The carbocyclyl group may be designated as “C₃₋₆ carbocyclyl” or similar designations. Examples of carbocyclyl rings include, but are not limited to, cyclopropyl, cyclobutyl, cyclopentyl, cyclohexyl, cyclohexenyl, 2,3-dihydro-indene, bicycle[2.2.2]octanyl, adamantyl, and spiro[4.4]nonanyl.

A “(carbocyclyl)alkyl” is a carbocyclyl group connected, as a substituent, via an alkylene group, such as “C₄₋₁₀ (carbocyclyl)alkyl” and the like, including but not limited to, cyclopropylmethyl, cyclobutylmethyl, cyclopropylethyl, cyclopropylbutyl, cyclobutylethyl, cyclopropylisopropyl, cyclopentylmethyl, cyclopentylethyl, cyclohexylmethyl, cyclohexylethyl, cycloheptylmethyl, and the like. In some cases, the alkylene group is a lower alkylene group.

As used herein, “cycloalkyl” means a fully saturated carbocyclyl ring or ring system. Examples include cyclopropyl, cyclobutyl, cyclopentyl, and cyclohexyl.

As used herein, “cycloalkenyl” means a carbocyclyl ring or ring system having at least one double bond, wherein no ring in the ring system is aromatic. An example is cyclohexenyl.

As used herein, “heterocyclyl” means a non-aromatic cyclic ring or ring system containing at least one heteroatom in the ring backbone. Heterocyclyls may be joined together in a fused, bridged or spiro-connected fashion. Heterocyclyls may have any degree of saturation provided that at least one ring in the ring system is not aromatic. The heteroatom(s) may be present in either a non-aromatic or aromatic ring in the ring system. The heterocyclyl group may have 3 to 20 ring members (i.e., the number of atoms making up the ring backbone, including carbon atoms and heteroatoms), although the present definition also covers the occurrence of the term “heterocyclyl” where no numerical range is designated. The heterocyclyl group may also be a medium size heterocyclyl having 3 to 10 ring members. The heterocyclyl group could also be a heterocyclyl having 3 to 6 ring members. The heterocyclyl group may be designated as “3-6 membered heterocyclyl” or similar designations. In preferred six membered monocyclic heterocyclyls, the heteroatom(s) are selected from one up to three of 0, N or S, and in preferred five membered monocyclic heterocyclyls, the heteroatom(s) are selected from one or two heteroatoms selected from O, N, or S. Examples of heterocyclyl rings include, but are not limited to, azepinyl, acridinyl, carbazolyl, cinnolinyl, dioxolanyl, imidazolinyl, imidazolidinyl, morpholinyl, oxiranyl, oxepanyl, thiepanyl, piperidinyl, piperazinyl, dioxopiperazinyl, pyrrolidinyl, pyrrolidonyl, pyrrolidionyl, 4-piperidonyl, pyrazolinyl, pyrazolidinyl, 1,3-dioxinyl, 1,3-dioxanyl, 1,4-dioxinyl, 1,4-dioxanyl, 1,3-oxathianyl, 1,4-oxathiinyl, 1,4-oxathianyl, 2H-1,2-oxazinyl, trioxanyl, hexahydro-1,3,5-triazinyl, 1,3-dioxolyl, 1,3-dioxolanyl, 1,3-dithiolyl, 1,3-dithiolanyl, isoxazolinyl, isoxazolidinyl, oxazolinyl, oxazolidinyl, oxazolidinonyl, thiazolinyl, thiazolidinyl, 1,3-oxathiolanyl, indolinyl, isoindolinyl, tetrahydrofuranyl, tetrahydropyranyl, tetrahydrothiophenyl, tetrahydrothiopyranyl, tetrahydro-1,4-thiazinyl, thiamorpholinyl, dihydrobenzofuranyl, benzimidazolidinyl, and tetrahydroquinoline.

A “(heterocyclyl)alkyl” is a heterocyclyl group connected, as a substituent, via an alkylene group. Examples include, but are not limited to, imidazolinylmethyl and indolinylethyl.

As used herein, “acyl” refers to —C(═O)R, wherein R is hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocycyl, as defined herein. Non-limiting examples include formyl, acetyl, propanoyl, benzoyl, and acryl.

An “O-carboxy” group refers to a “—OC(═O)R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocycyl, as defined herein.

A “C-carboxy” group refers to a “—C(═O)OR” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocycyl, as defined herein. A non-limiting example includes carboxyl (i.e., —C(═O)OH).

A “cyano” group refers to a “—CN” group.

A “cyanato” group refers to an “—OCN” group.

An “isocyanato” group refers to a “—NCO” group.

A “thiocyanato” group refers to a “—SCN” group.

An “isothiocyanato” group refers to an “—NCS” group.

A “sulfinyl” group refers to an “—S(═O)R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocycyl, as defined herein.

A “sulfonyl” group refers to an “—SO₂R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocycyl, as defined herein.

An “S-sulfonamido” group refers to a “—SO₂NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocycyl, as defined herein.

An “N-sulfonamido” group refers to a “—N(R_{A})SO₂R_{B}” group in which R_{A} and R_{b} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocycyl, as defined herein.

An “O-carbamyl” group refers to a “—OC(═O)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocycyl, as defined herein.

An “N-carbamyl” group refers to an “—N(R_{A})C(═O)OR_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocycyl, as defined herein.

An “O-thiocarbamyl” group refers to a “—OC(═S)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocycyl, as defined herein.

An “N-thiocarbamyl” group refers to an “—N(R_{A})C(═S)OR_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocycyl, as defined herein.

A “C-amido” group refers to a “—C(═O)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocycyl, as defined herein.

An “N-amido” group refers to a “—N(R_{A})C(═O)R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocycyl, as defined herein.

An “amino” group refers to a “—NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocycyl, as defined herein. A non-limiting example includes free amino (i.e., —NH₂).

An “aminoalkyl” group refers to an amino group connected via an alkylene group.

An “alkoxyalkyl” group refers to an alkoxy group connected via an alkylene group, such as a “C₂₋₈ alkoxyalkyl” and the like.

As used herein, a substituted group is derived from the unsubstituted parent group in which there has been an exchange of one or more hydrogen atoms for another atom or group. Unless otherwise indicated, when a group is deemed to be “substituted,” it is meant that the group is substituted with one or more substituents independently selected from C₁-C₆ alkyl, C₁-C₆ alkenyl, C₁-C₆ alkynyl, C₁-C₆ heteroalkyl, C₃-C₇ carbocyclyl (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkyl (optionally substituted with halo, C₁-C₆ alkyl, C₁-C₆ alkoxy, C₁-C₆ haloalkyl, and C₁-C₆ haloalkoxy), 3-10 membered heterocycyl (optionally substituted with halo, C₁-C₆ alkyl, C₁-C₆ alkoxy, C₁-C₆ haloalkyl, and C₁-C₆ haloalkoxy), 3-10 membered heterocycyl-C₁-C₆-alkyl (optionally substituted with halo, C₁-C₆ alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆ haloalkyl, and C₁-C₆ haloalkoxy), aryl(C₁-C₆)alkyl (optionally substituted with halo, C₁-C₆ alkyl, C₁-C₆ alkoxy, C₁-C₆ haloalkyl, and C₁-C₆ haloalkoxy), 5-10 membered heteroaryl (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkyl (optionally substituted with halo, C₁-C₆ alkyl, C₁-C₆ alkoxy, C₁-C₆ haloalkyl, and C₁-C₆ haloalkoxy), halo, cyano, hydroxy, C₁-C₆ alkoxy, C₁-C₆ alkoxy(C₁-C₆)alkyl (i.e., ether), aryloxy (optionally substituted with halo, C₁-C₆ alkyl, C₁-C₆ alkoxy, C₁-C₆ haloalkyl, and C₁-C₆ haloalkoxy), C₃-C₇ carbocyclyloxy (optionally substituted with halo, C₁-C₆ alkyl, C₁-C₆ alkoxy, C₁-C₆ haloalkyl, and C₁-C₆ haloalkoxy), 3-10 membered heterocyclyl-oxy (optionally substituted with halo, C₁-C₆ alkyl, C₁-C₆ alkoxy, C₁-C₆ haloalkyl, and C₁-C₆ haloalkoxy), 5-10 membered heteroaryl-oxy (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkoxy (optionally substituted with halo, C₁-C₆ alkyl, C₁-C₆ alkoxy, C₁-C₆ haloalkyl, and C₁-C₆ haloalkoxy), 3-10 membered heterocyclyl-C₁-C₆-alkoxy (optionally substituted with halo, C₁-C₆ alkyl, C₁-C₆ alkoxy, C₁-C₆ haloalkyl, and C₁-C₆ haloalkoxy), aryl(C₁-C₆)alkoxy (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkoxy (optionally substituted with halo, C₁-C₆ alkyl, C₁-C₆ alkoxy, C₁-C₆ haloalkyl, and C₁-C₆ haloalkoxy), sulfhydryl (mercapto), halo(C₁-C₆)alkyl (e.g., —CF₃), halo(C₁-C₆)alkoxy (e.g., —OCF₃), C₁-C₆ alkylthio, arylthio (optionally substituted with halo, C₁-C₆ alkyl, C₁-C₆ alkoxy, C₁-C₆ haloalkyl, and C₁-C₆ haloalkoxy), C₃-C₇ carbocyclylthio (optionally substituted with halo, C₁-C₆ alkyl, C₁-C₆ alkoxy, C₁-C₆ haloalkyl, and C₁-C₆ haloalkoxy), 3-10 membered heterocyclyl-thio (optionally substituted with halo, C₁-C₆ alkyl, C₁-C₆ alkoxy, C₁-C₆ haloalkyl, and C₁-C₆ haloalkoxy), 5-10 membered heteroaryl-thio (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkylthio (optionally substituted with halo, C₁-C₆ alkyl, C₁-C₆ alkoxy, C₁-C₆ haloalkyl, and C₁-C₆ haloalkoxy), 3-10 membered heterocyclyl-C₁-C₆-alkylthio (optionally substituted with halo, C₁-C₆ alkyl, C₁-C₆ alkoxy, C₁-C₆ haloalkyl, and C₁-C₆ haloalkoxy), aryl(C₁-C₆)alkylthio (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkylthio (optionally substituted with halo, C₁-C₆ alkyl, C₁-C₆ alkoxy, C₁-C₆ haloalkyl, and C₁-C₆ haloalkoxy), amino, amino(C₁-C₆)alkyl, nitro, O-carbamyl, N-carbamyl, O-thiocarbamyl, N-thiocarbamyl, C-amido, N-amido, S-sulfonamido, N-sulfonamido, C-carboxy, O-carboxy, acyl, cyanato, isocyanato, thiocyanato, isothiocyanato, sulfinyl, sulfonyl, and oxo (═O). Wherever a group is described as “optionally substituted” that group can be substituted with the above substituents.

It is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂—, and the like. Other radical naming conventions clearly indicate that the radical is a di-radical such as “alkylene” or “alkenylene.”

Wherever a substituent is depicted as a di-radical (i.e., has two points of attachment to the rest of the molecule), it is to be understood that the substituent can be attached in any directional configuration unless otherwise indicated. Thus, for example, a substituent depicted as -AE- or

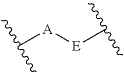

includes the substituent being oriented such that the A is attached at the leftmost attachment point of the molecule as well as the case in which A is attached at the rightmost attachment point of the molecule.

### Compositions

The present disclosure provides topical compositions comprising compounds that provide a cooling sensation and/or are modulators of TRPM8.

In some embodiments, the topical composition can comprise a carrier and a compound of Formula (I):

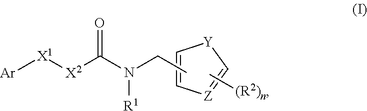

or a salt or solvate thereof;
wherein
Ar is optionally substituted aryl, optionally substituted carbocyclyl, or optionally substituted heteroaryl;
Y is oxygen or sulfur;
Z is nitrogen or CR;
R is hydrogen or lower alkyl;
X¹-X² is O—CR^{2a}R^{2b}, CHR³—CHR⁴, or CR⁵═CR⁶;
R^{2a}, R^{2b}, R³, R⁴, R⁵, and R⁶ are independently hydrogen or lower alkyl;
R¹ is an optionally substituted five-membered heteroaryl;
n is 0, 1, 2, or 3; and
each R² is independently optionally substituted alkyl, optionally substituted heteroalkyl, optionally substituted alkenyl, alkoxy, hydroxyl, amino, N-alkyl amino, N-dialkyl amino, halo, nitro, cyano, acyl, carboxyl, carboxyl ester, or amide,
wherein each optional substituent is selected from the group consisting of alkyl, heteroalkyl, alkenyl, alkoxy, hydroxyl, amino, N-alkyl amino, N-dialkyl amino, halo, nitro, cyano, acyl, carboxyl, carboxyl ester, or amide; or two substituents, together with the atoms to which they are attached, form a carbocyclyl optionally substituted with alkyl or alkoxy; or two substituents, together with the atoms to which they are attached, form a heterocyclyl containing one or more heteroatom(s) selected from nitrogen, oxygen, and sulfur.

Examples of compounds of Formula (I) suitable for use in a topical compositions include, but are not limited to, the following structures:

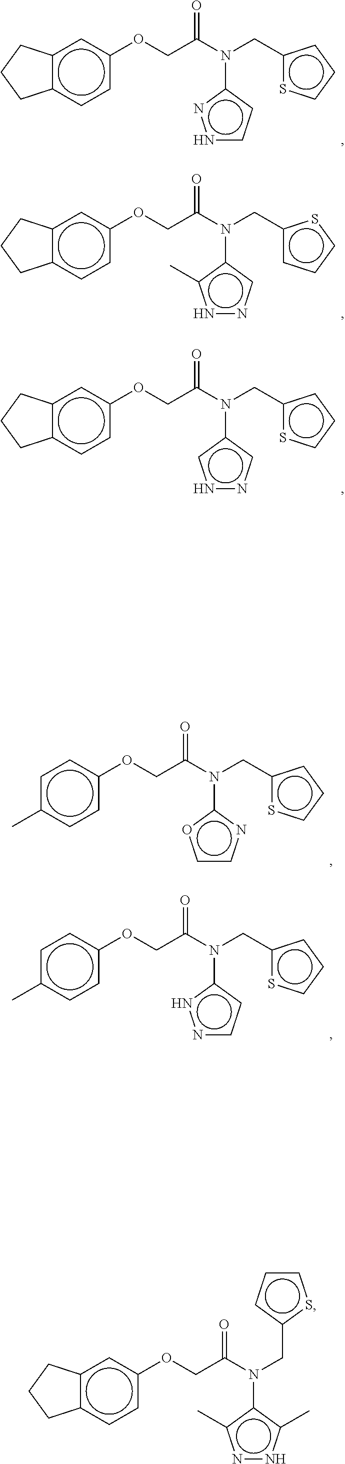

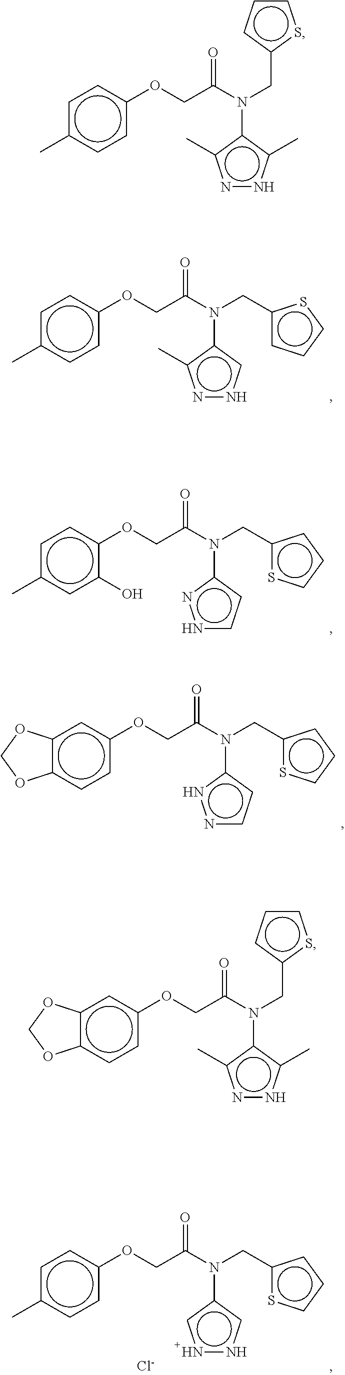

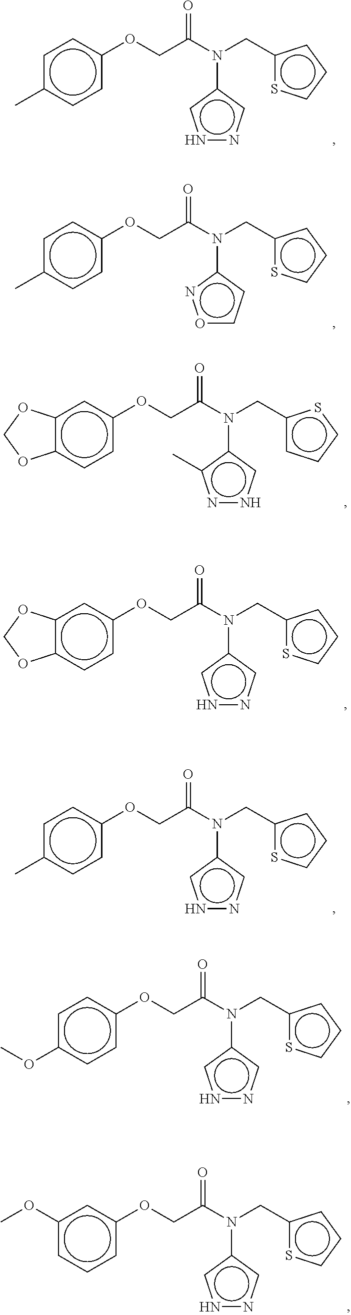

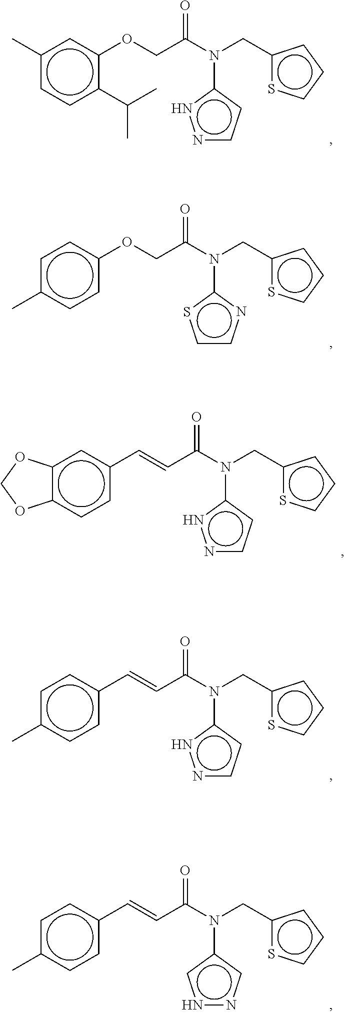

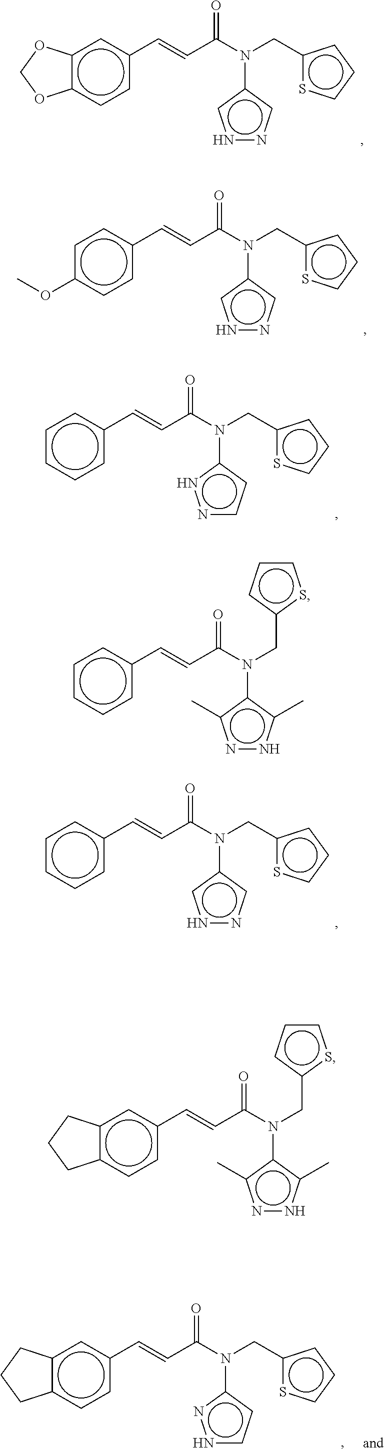

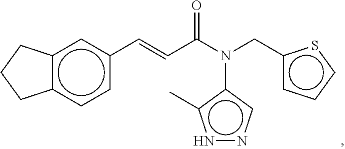

or a salt or solvate thereof.

In some embodiments the compound of Formula (I) can be

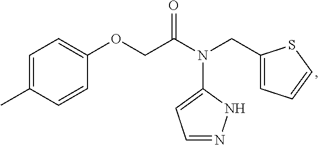 [SMILES: Cc1ccc(OCC(=O)N(Cc2cccs2)c2ccn[nH]2)cc1]

or a salt or solvate thereof.

In some embodiments the compound of Formula (I) can be selected from

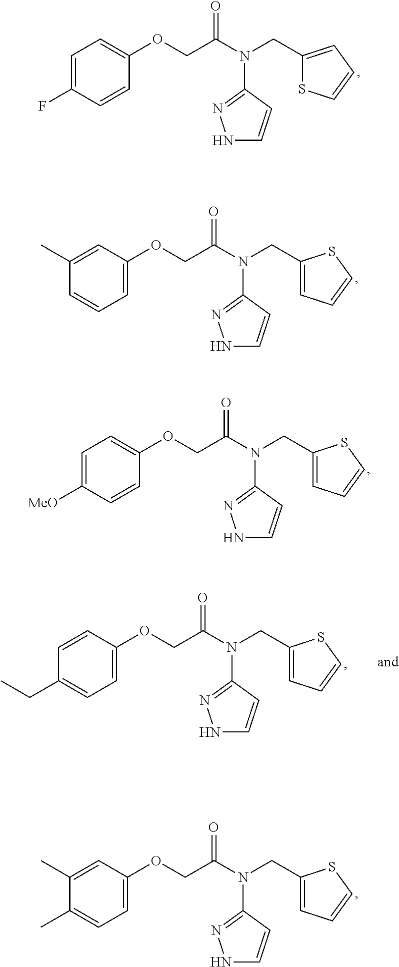 [SMILES: O=C(COc1ccc(F)cc1)N(Cc1cccs1)c1cc[nH]n1 ; Cc1cccc(OCC(=O)N(Cc2cccs2)c2cc[nH]n2)c1 ; COc1ccc(OCC(=O)N(Cc2cccs2)c2cc[nH]n2)cc1 ; CCc1ccc(OCC(=O)N(Cc2cccs2)c2cc[nH]n2)cc1 ; Cc1ccc(OCC(=O)N(Cc2cccs2)c2cc[nH]n2)cc1C]

or a salt or solvate thereof.

In some embodiments, the topical composition can comprise a carrier and a compound of Formula (IIa) or (IIb):

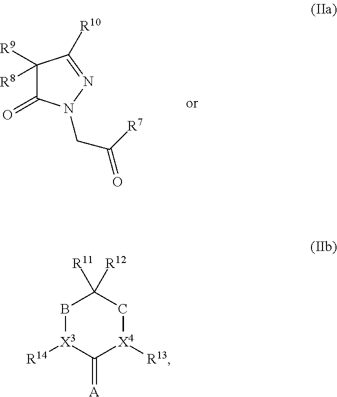

or a salt or solvate thereof;
wherein
   R⁷ is optionally substituted aryl, optionally substituted carbocyclyl, optionally substituted heteroaryl, or optionally substituted heterocyclyl;
   R⁸ and R⁹ are independently selected from the group consisting of optionally substituted alkyl, optionally substituted alkenyl, optionally substituted aryl, optionally substituted carbocyclyl, optionally substituted heteroaryl, and optionally substituted heterocyclyl;
   R¹⁰ is optionally substituted alkyl, optionally substituted alkenyl, optionally substituted aryl, optionally substituted carbocyclyl, optionally substituted heteroaryl, or optionally substituted heterocyclyl;
   either R¹¹ or R¹² is optionally substituted C₁-C₃ alkyl; and the remaining R¹¹ or R¹² is selected from the group consisting of optionally substituted alkyl, optionally substituted alkenyl, optionally substituted alkynyl, optionally substituted alkoxy, optionally substituted alkylaryl, optionally substituted alkoxyaryl, optionally substituted aryl, optionally substituted aryloxy, optionally substituted heteroalkyl, optionally substituted carbocyclyl, or optionally substituted heterocylcyl; or alternatively, R¹¹ and R¹², taken together with the atoms to which they are attached, form an optionally substituted carbocyclyl;
   X³ and X⁴ are independently CH or N; provided that X³ and X⁴ are not both CH;
   R¹³ and R¹⁴ are independently selected from the group consisting of hydrogen, halogen, hydroxyl, cyano, carboxy, optionally substituted C₁-C₈ alkyl, optionally substituted alkenyl, optionally substituted alkynyl, optionally substituted heteroalkyl, optionally substituted alkylaryl, optionally substituted alkoxyaryl, optionally substituted aryl, optionally substituted aryloxy, optionally substituted heteroaryl, optionally substituted heteroaryloxy, optionally substituted carbocyclyl, or optionally substituted heterocyclyl, -alkylene-carbonyl-aryl, -alkylene-carbonyl-heteroaryl, -alkylene-carbonyl-(substituted aryl), -alkylene-carbonyl-(substituted heteroaryl), -alkylene-carbonyl-O-aryl, -alkylene-carbonyl-O-(substituted aryl), -alkylene-carbonyl-NR¹⁵-aryl, -alkylene-carbonyl-NR¹⁵-(substituted aryl), -alkylene-carbonyl-O-heteroaryl, -alkylene-carbonyl-O-(substituted heteroaryl), -alkylene-carbonyl-NR¹⁵-heteroaryl, -alkylene-carbonyl-NR¹⁵-(substituted heteroaryl), OR¹⁵, and NR¹⁵R¹⁶; or alternatively, X³ and R¹⁴, or X⁴ and R¹³, taken together, is independently O or S;
   A is O, S, or NR¹¹;
   B and C are independently CH₂, C═O, or a covalent bond; provided that B and C are not both covalent bonds; and
   R¹⁵ and R¹⁶ are independently selected from the group consisting of hydrogen, optionally substituted alkyl, optionally substituted aryl, optionally substituted arylalkyl, optionally substituted heteroalkyl, optionally substituted heteroaryl, optionally substituted heteroarylalkyl, optionally substituted acylamido, and optionally substituted diacylamido; or alternatively, R⁹ and R¹⁰, together with the atoms to which they are bonded, form an optionally substituted cycloheteroalkyl.

Examples of suitable compounds of Formula (IIa) or (IIb) for use in a topical composition include, but are not limited to, the following structures:

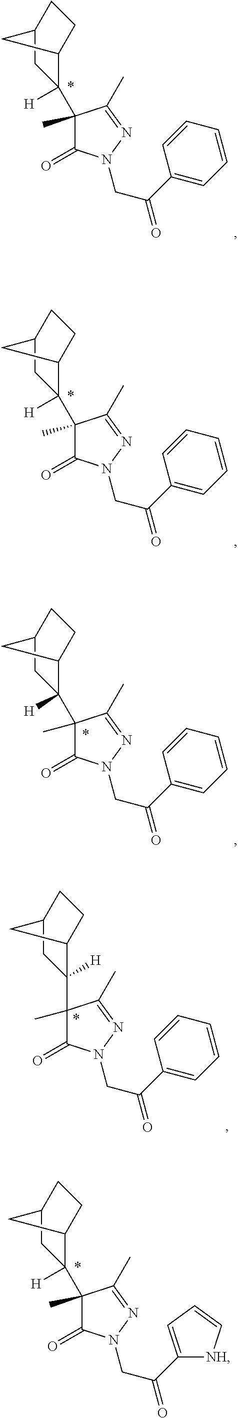

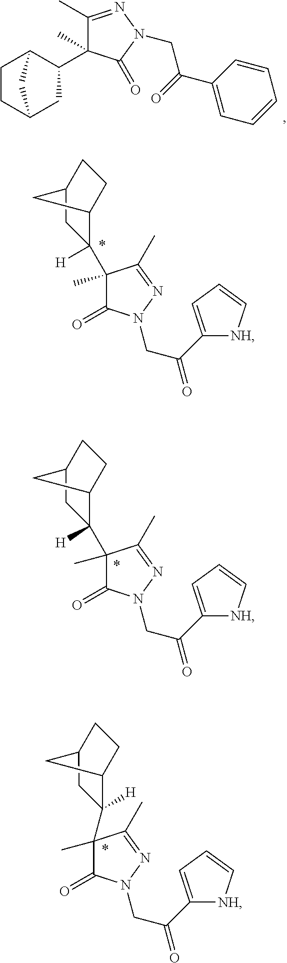 [SMILES: CC1=NN(CC(=O)c2ccccc2)C(=O)[C@@]1(C)[C@@H]1C[C@H]2CC[C@@H]1C2 ; CC1=NN(CC(=O)c2ccc[nH]2)C(=O)[C@@]1(C)C1CC2CCC1C2 ; CC1=NN(CC(=O)c2ccc[nH]2)C(=O)C1(C)[C@@H]1CC2CCC1C2 ; CC1=NN(CC(=O)c2ccc[nH]2)C(=O)C1(C)[C@H]1CC2CCC1C2]

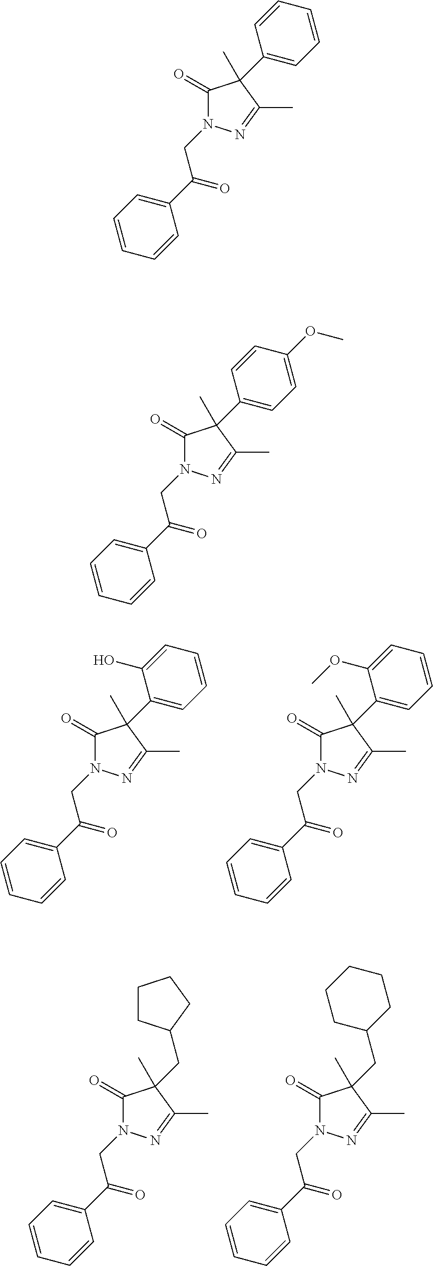 [SMILES: CC1=NN(CC(=O)c2ccccc2)C(=O)C1(C)c1ccccc1 ; COc1ccc(C2(C)C(=O)N(CC(=O)c3ccccc3)N=C2C)cc1 ; CC1=NN(CC(=O)c2ccccc2)C(=O)C1(C)c1ccccc1O ; COc1ccccc1C1(C)C(=O)N(CC(=O)c2ccccc2)N=C1C ; CC1=NN(CC(=O)c2ccccc2)C(=O)C1(C)CC1CCCC1 ; CC1=NN(CC(=O)c2ccccc2)C(=O)C1(C)CC1CCCCC1]

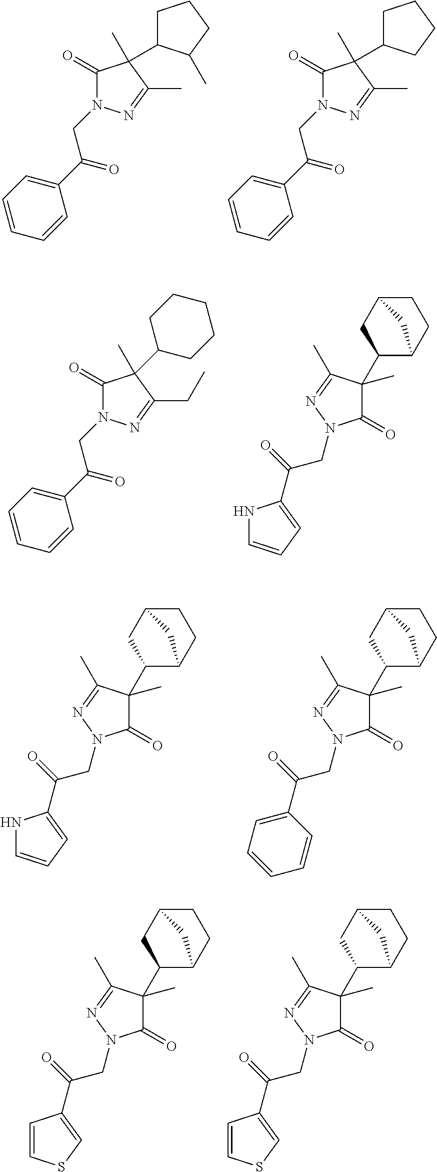

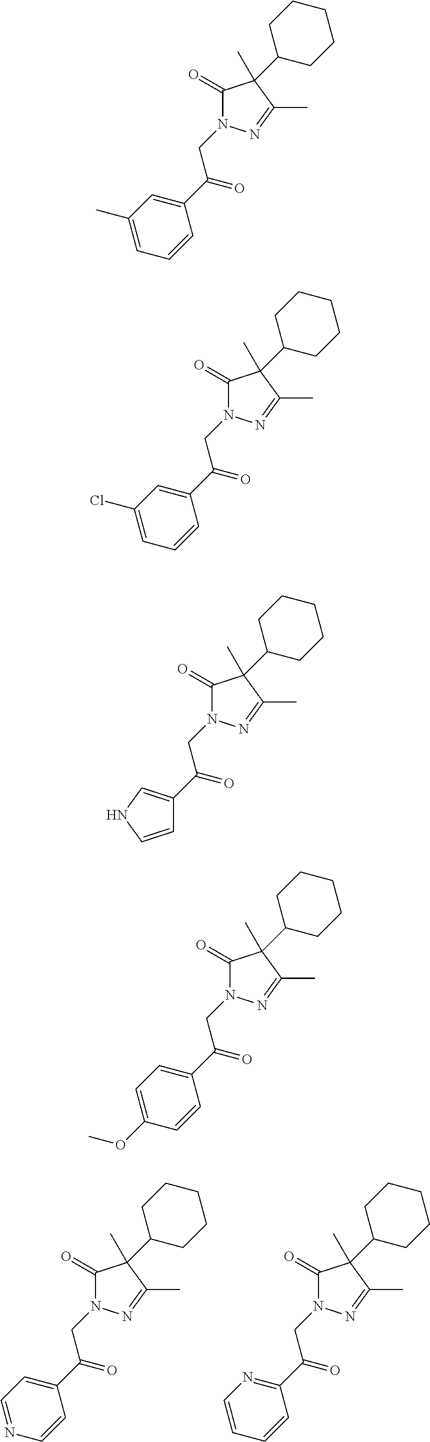 [SMILES: CC1=NN(CC(=O)c2cccc(C)c2)C(=O)C1(C)C1CCCCC1 ; CC1=NN(CC(=O)c2cccc(Cl)c2)C(=O)C1(C)C1CCCCC1 ; CC1=NN(CC(=O)c2cc[nH]c2)C(=O)C1(C)C1CCCCC1 ; COc1ccc(C(=O)CN2N=C(C)C(C)(C3CCCCC3)C2=O)cc1 ; CC1=NN(CC(=O)c2ccncc2)C(=O)C1(C)C1CCCCC1 ; CC1=NN(CC(=O)c2ccccn2)C(=O)C1(C)C1CCCCC1]

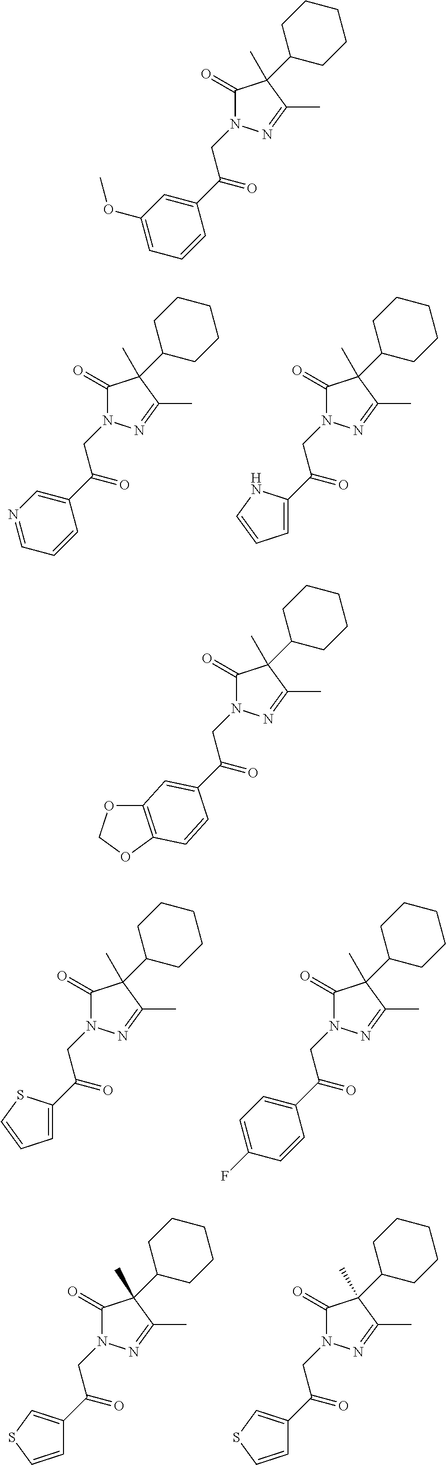

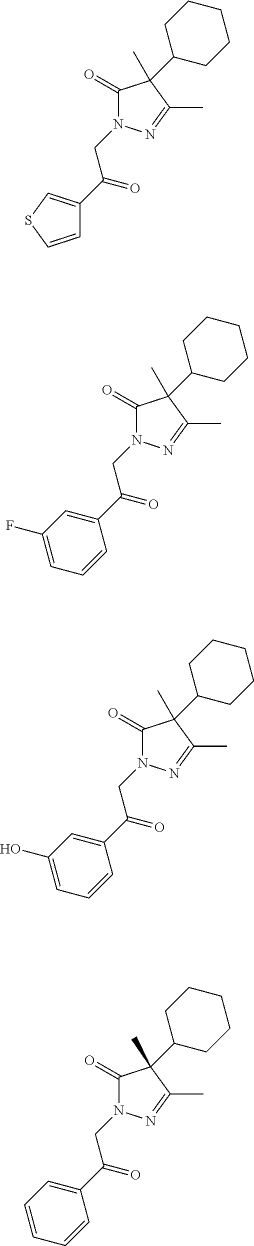 [SMILES: CC1=NN(CC(=O)c2ccsc2)C(=O)C1(C)C1CCCCC1 ; CC1=NN(CC(=O)c2cccc(F)c2)C(=O)C1(C)C1CCCCC1 ; CC1=NN(CC(=O)c2cccc(O)c2)C(=O)C1(C)C1CCCCC1 ; CC1=NN(CC(=O)c2ccccc2)C(=O)[C@]1(C)C1CCCCC1]

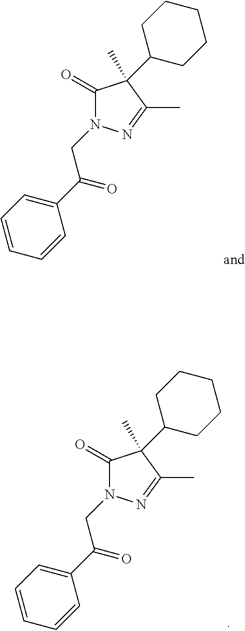 [SMILES: CC1=NN(CC(=O)c2ccccc2)C(=O)[C@@]1(C)C1CCCCC1 ; "and" ; CC1=NN(CC(=O)c2ccccc2)C(=O)[C@@]1(C)C1CCCCC1]

Further examples of suitable compounds of Formula (IIa) or (IIb) include, but are not limited to, the following structures:

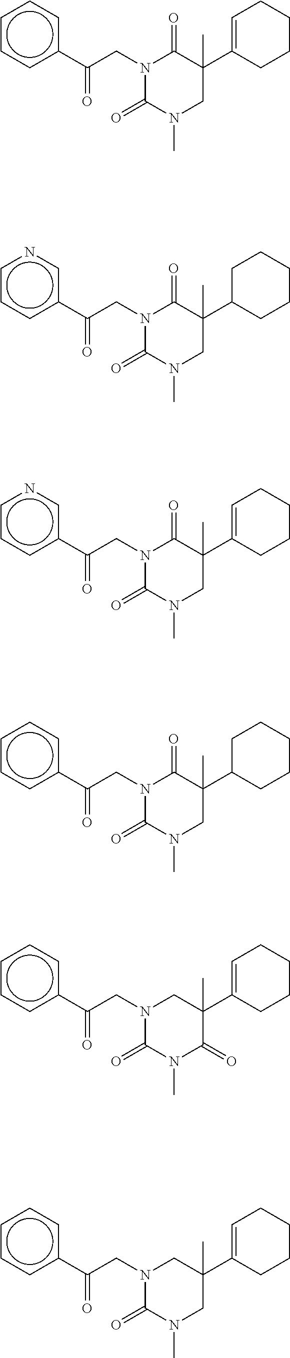

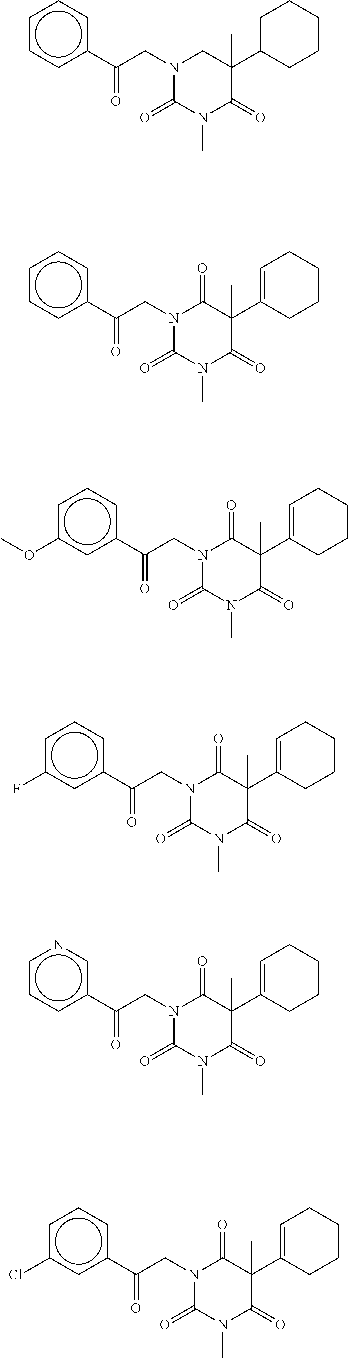

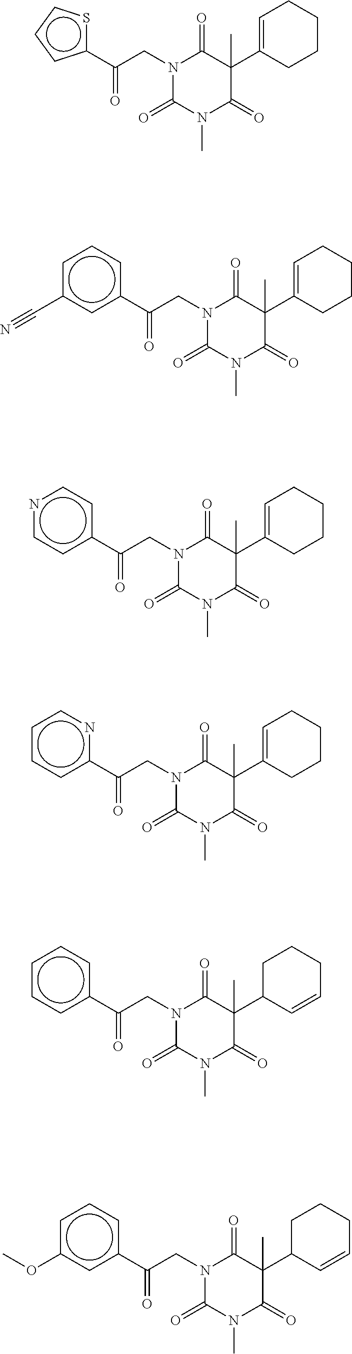

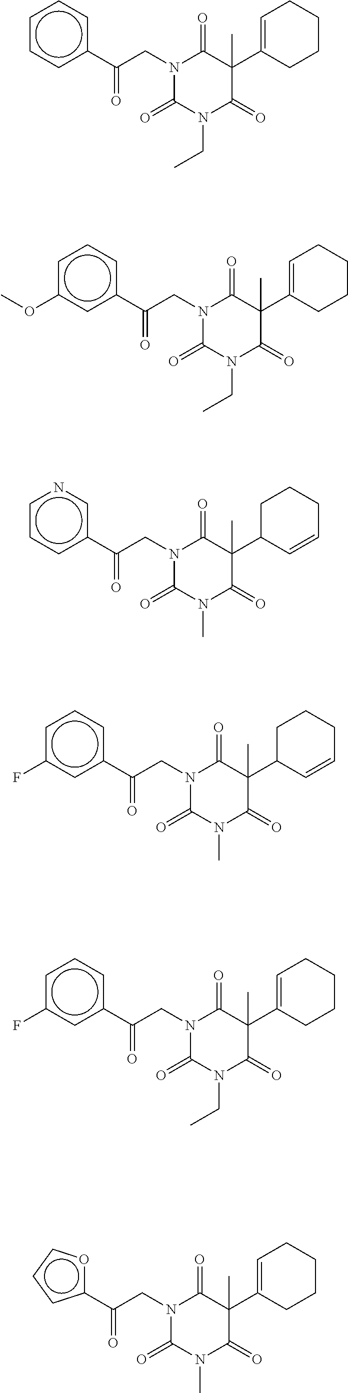

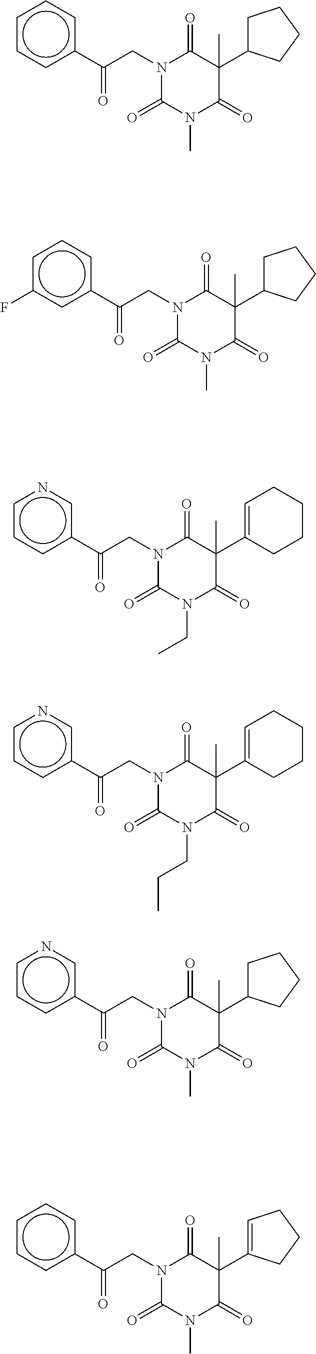

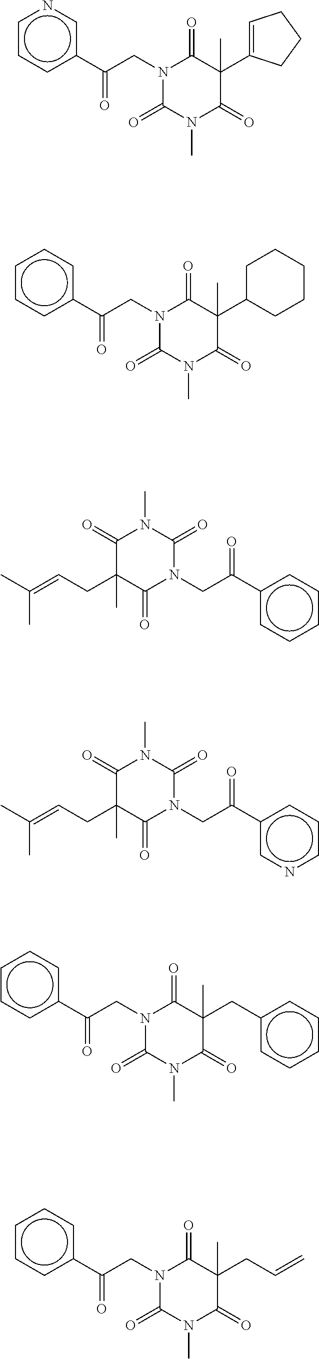

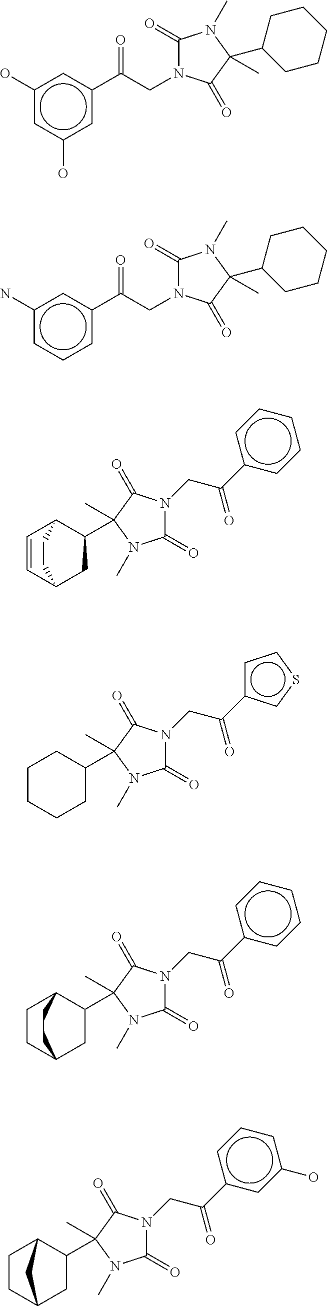

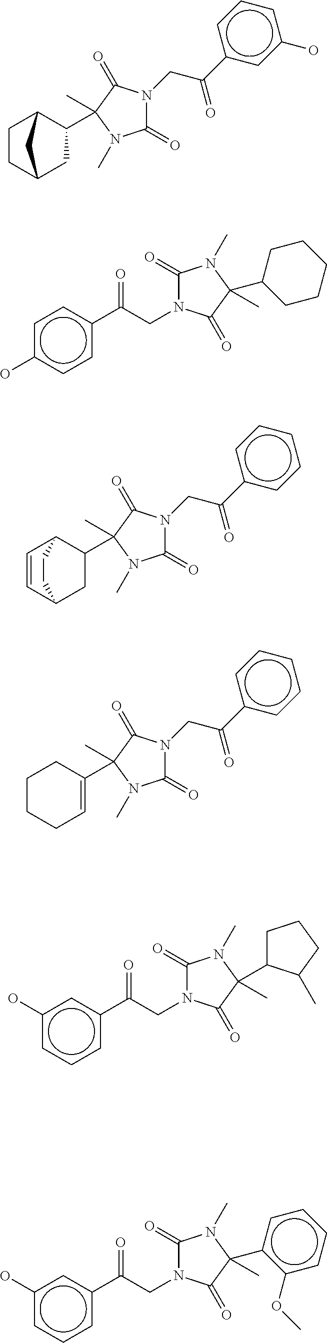

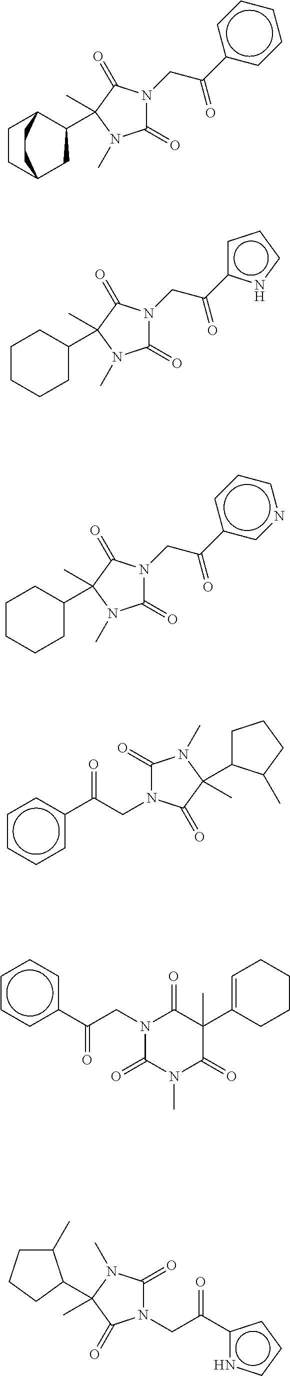

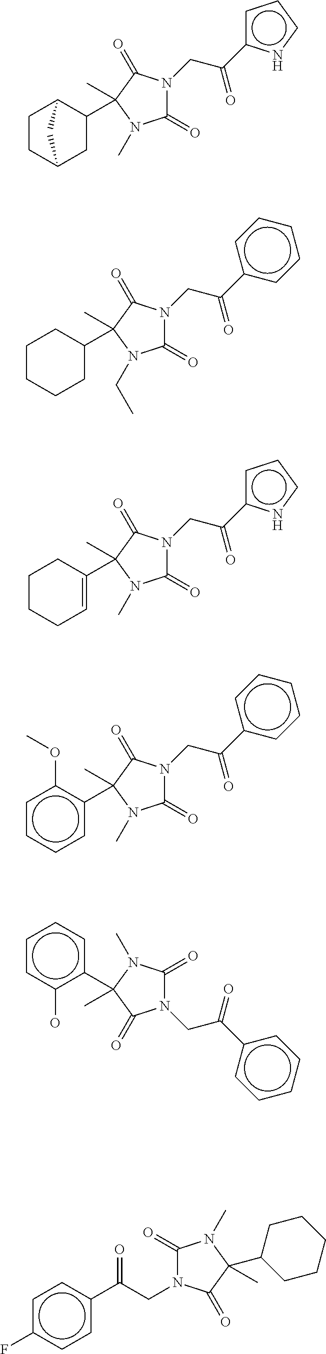

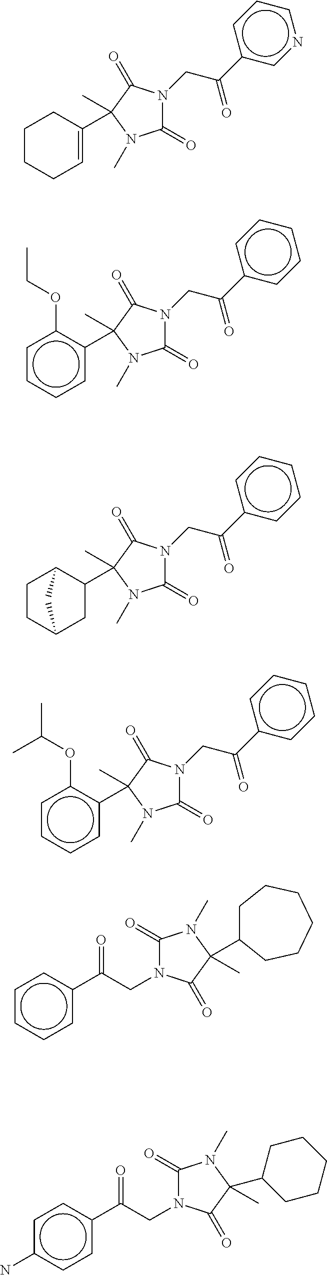

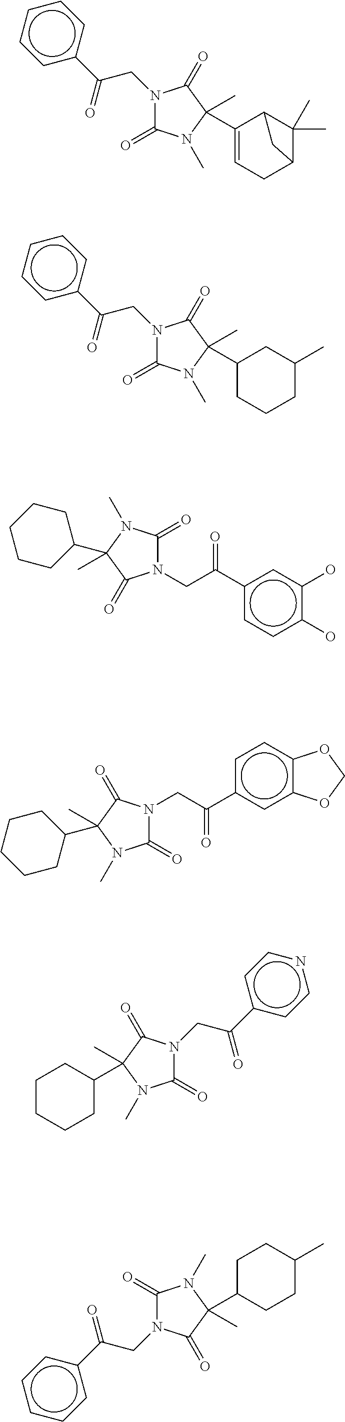

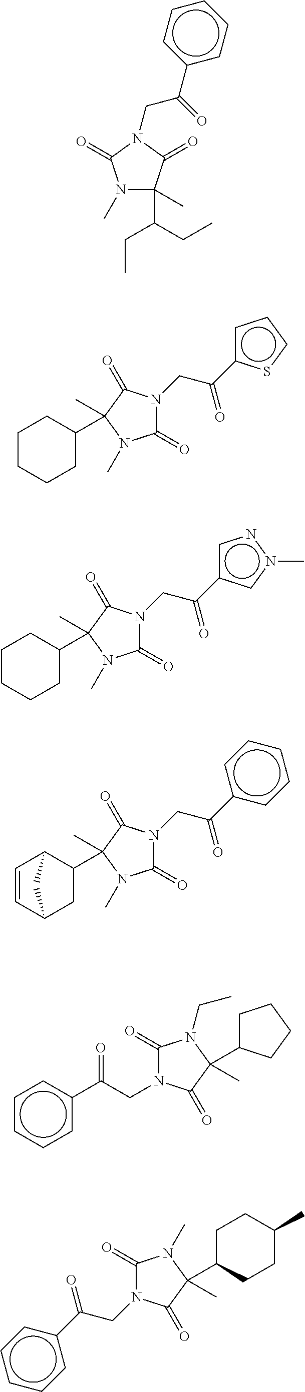

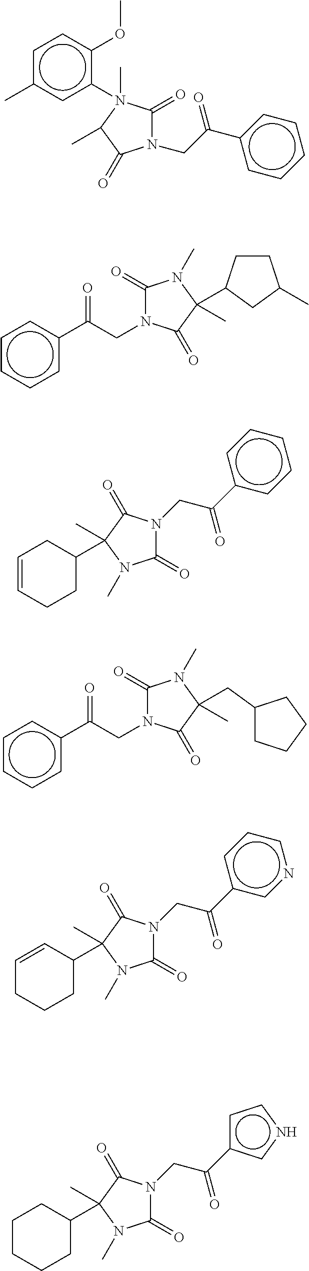

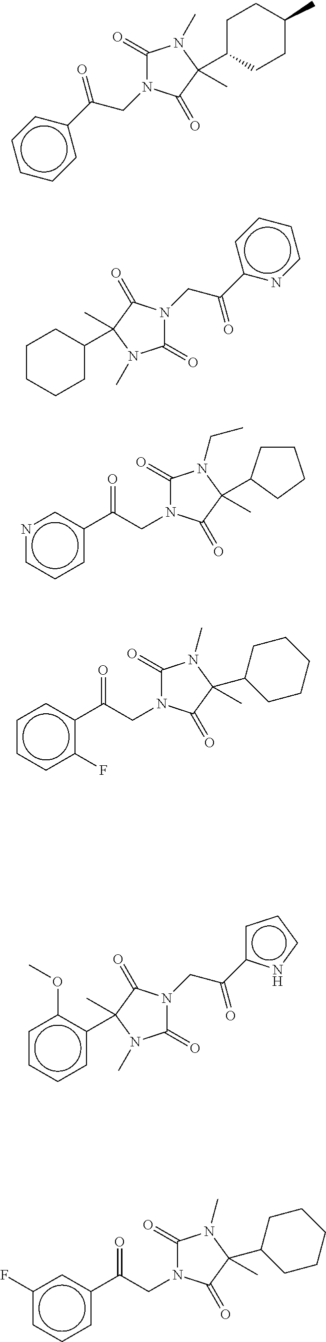

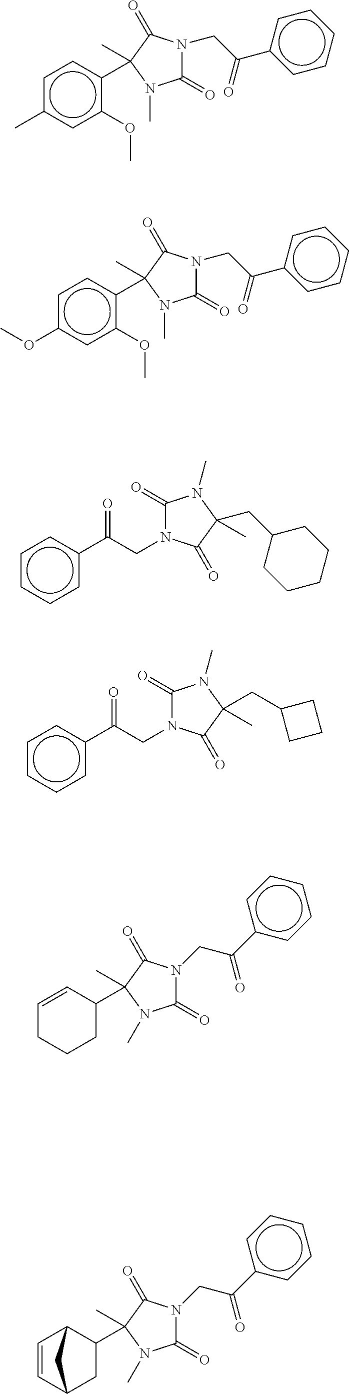

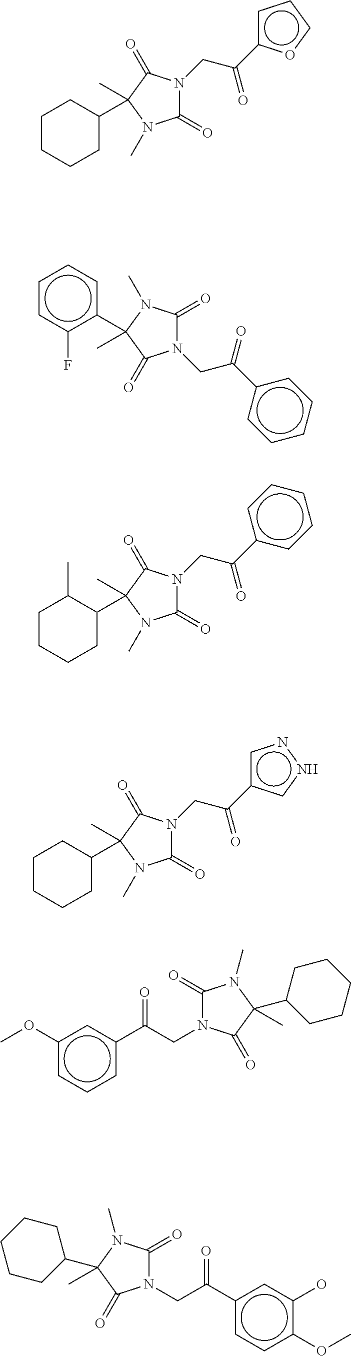

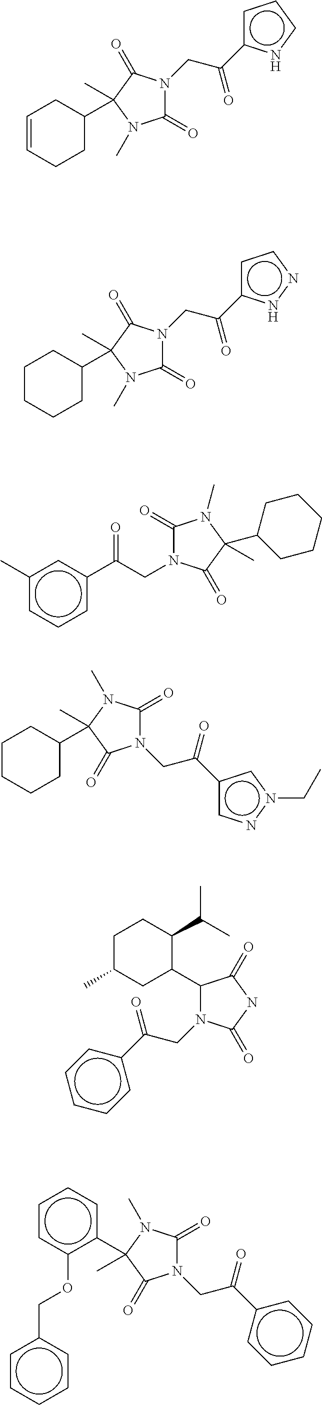

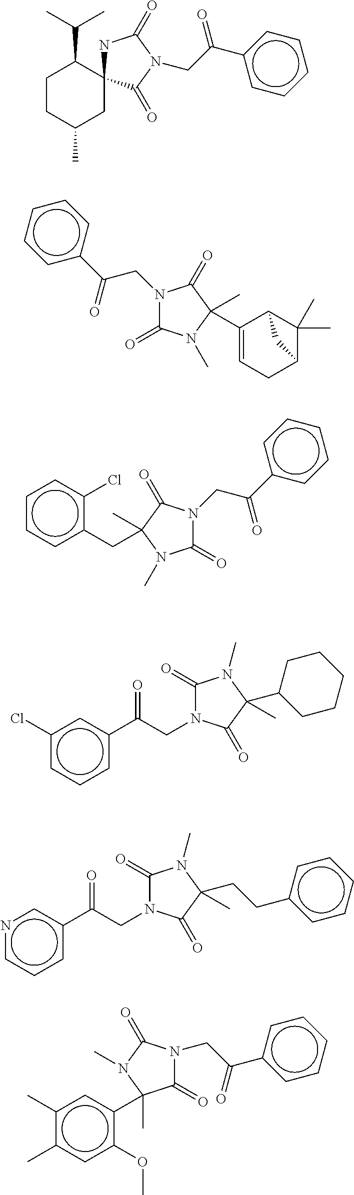

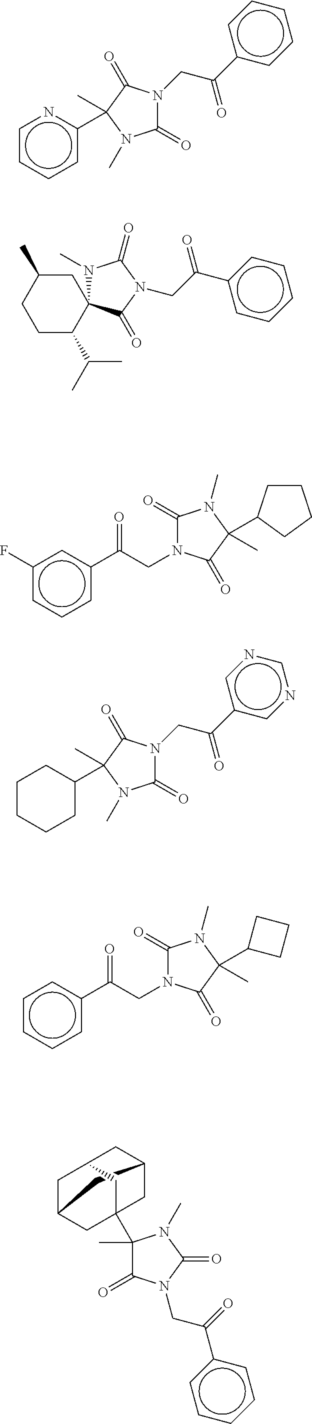

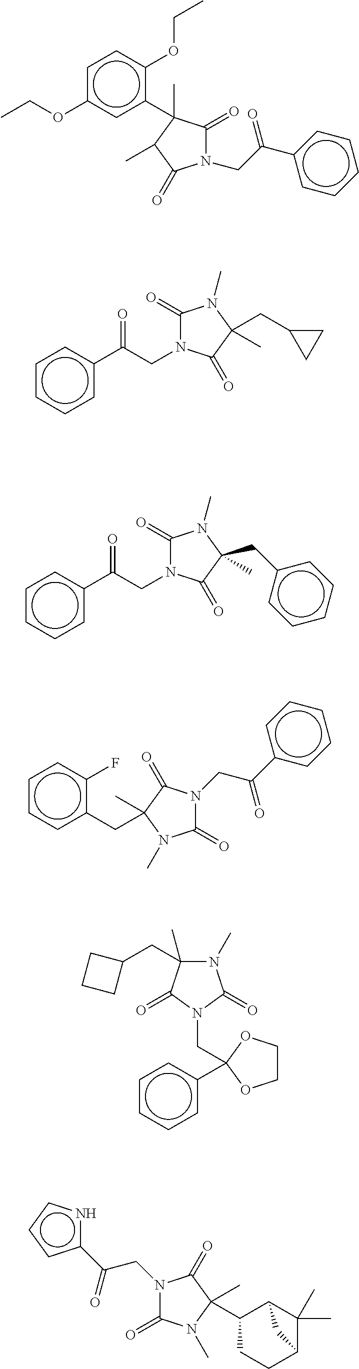

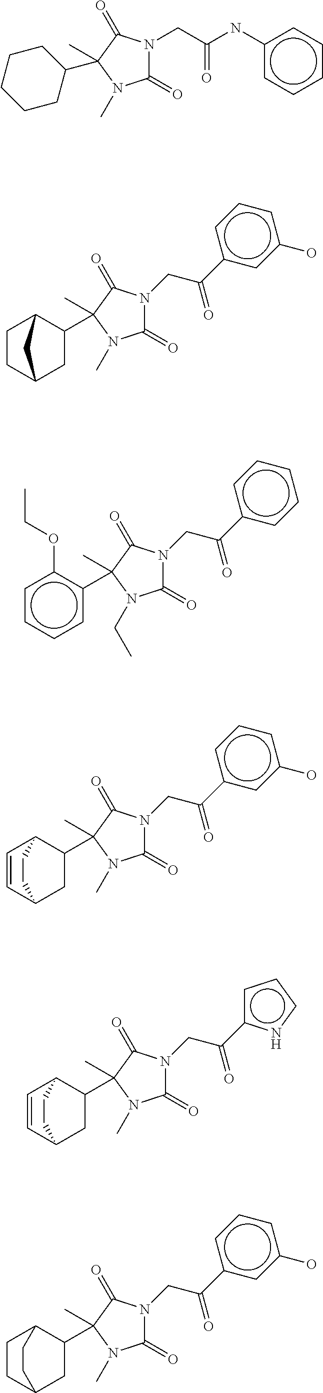

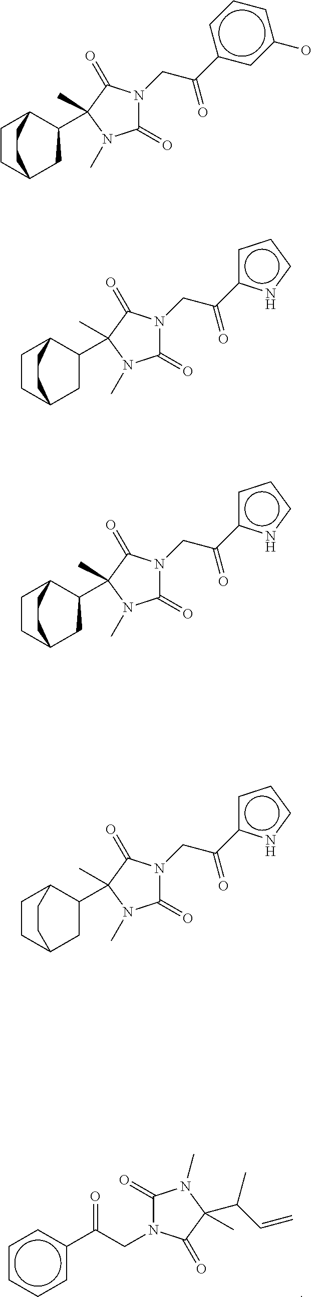

In some embodiments, the topical composition can comprise a carrier and a compound of Formula (III):

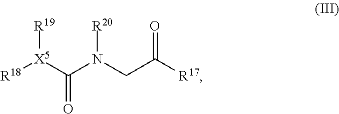

or a salt or solvate thereof;
wherein
X⁵ is CR²¹ or N;
R¹⁷ is optionally substituted aryl, optionally substituted carbocyclyl, optionally substituted heteroaryl, or optionally substituted heterocyclyl;
R¹⁸ and R¹⁹ are the same or different and are independently selected form the group consisting of hydrogen, optionally substituted alkyl, optionally substituted alkenyl, optionally substituted aryl, optionally substituted carbocyclyl, optionally substituted heteroaryl, and optionally substituted heterocyclyl;
R²⁰ is hydrogen, hydroxyl, alkoxy, optionally substituted alkyl, optionally substituted alkenyl, optionally substituted aryl, optionally substituted carbocyclyl, optionally substituted heteroaryl, or optionally substituted heterocyclyl; and
R²¹ is hydrogen, optionally substituted alkyl, optionally substituted alkenyl, optionally substituted aryl, optionally substituted carbocyclyl, optionally substituted heteroaryl, or optionally substituted heterocyclyl; or alternatively, R¹⁹ and R²¹, or R¹⁹ and R²⁰, taken together with the atoms to which they are attached, form an optionally substituted carbocyclyl or optionally substituted heterocyclyl.

Examples of suitable compounds of Formula (III) for use in a topical composition include, but are not limited to, the following structures:

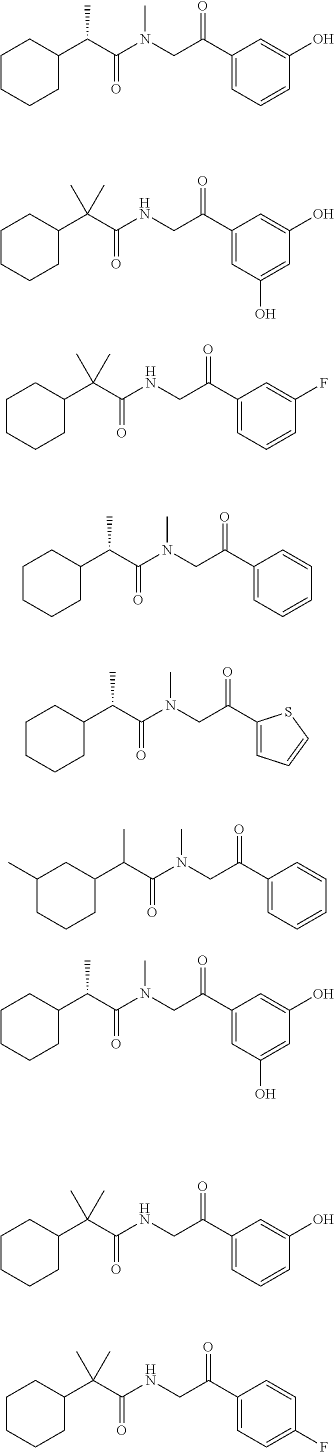

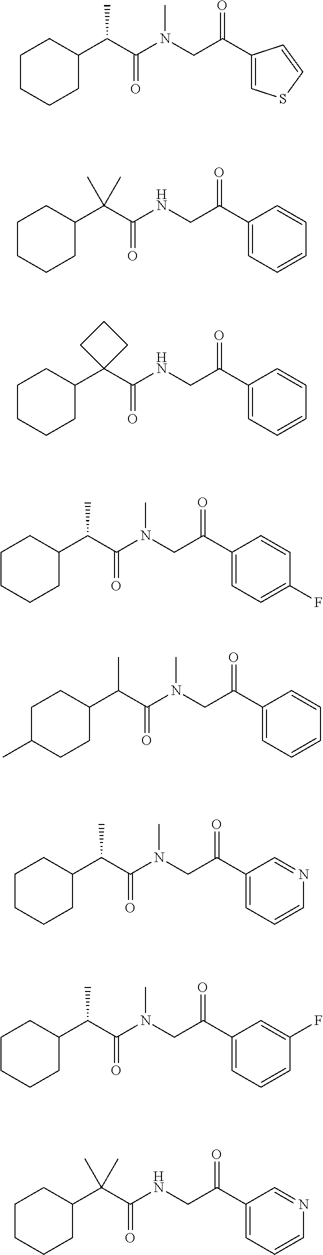

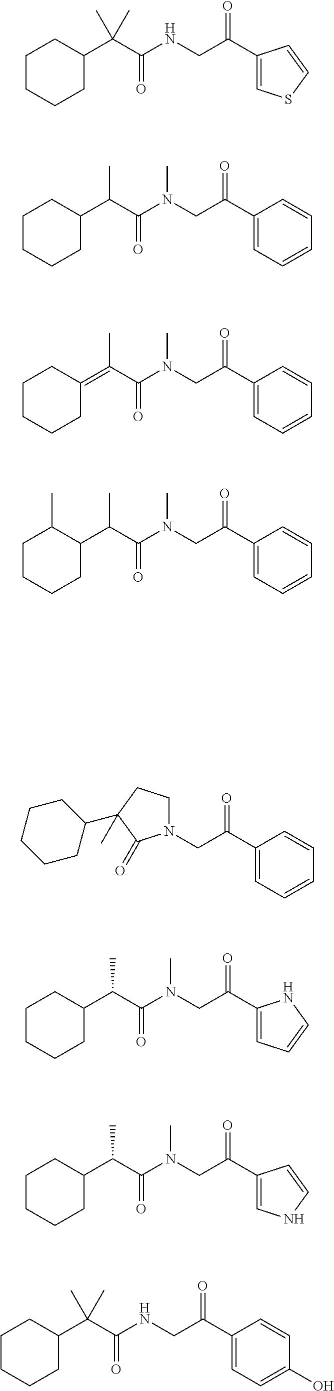

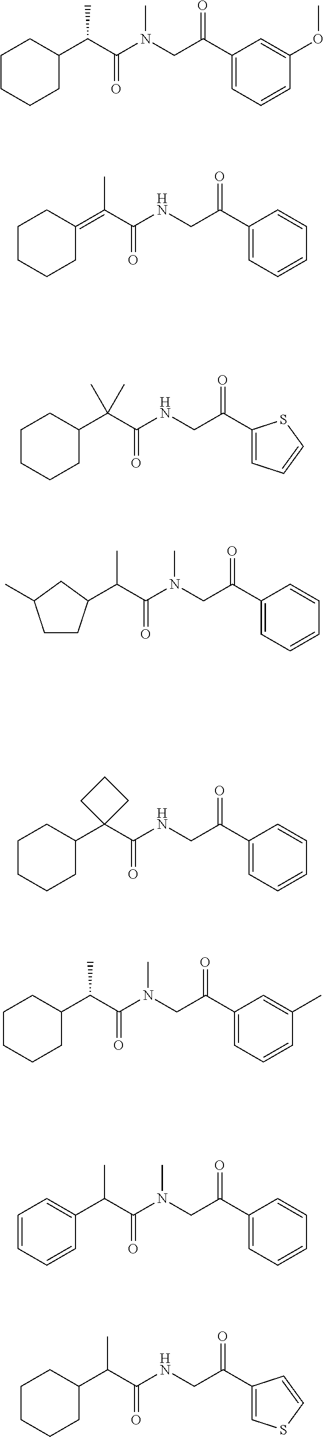

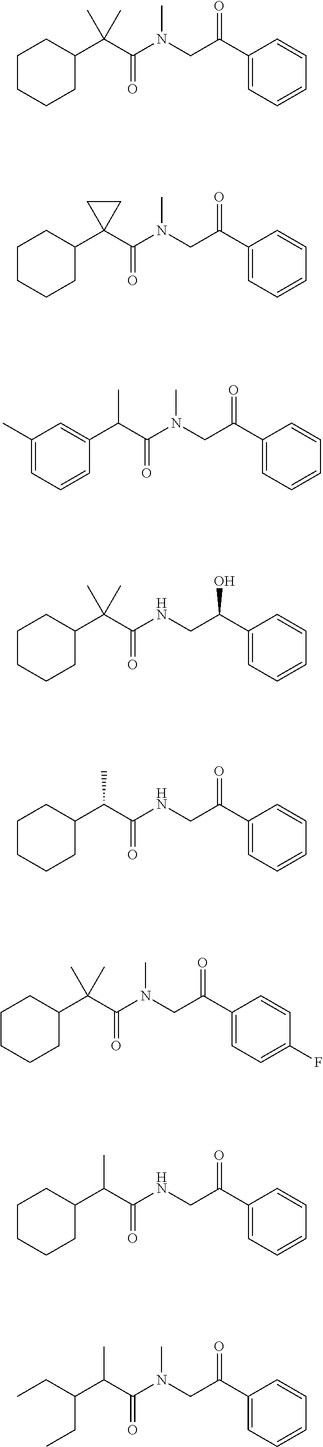

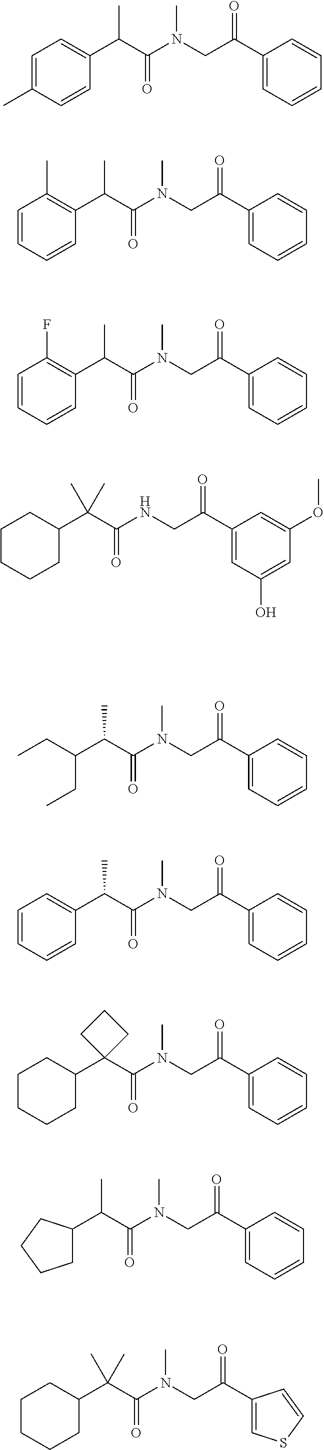

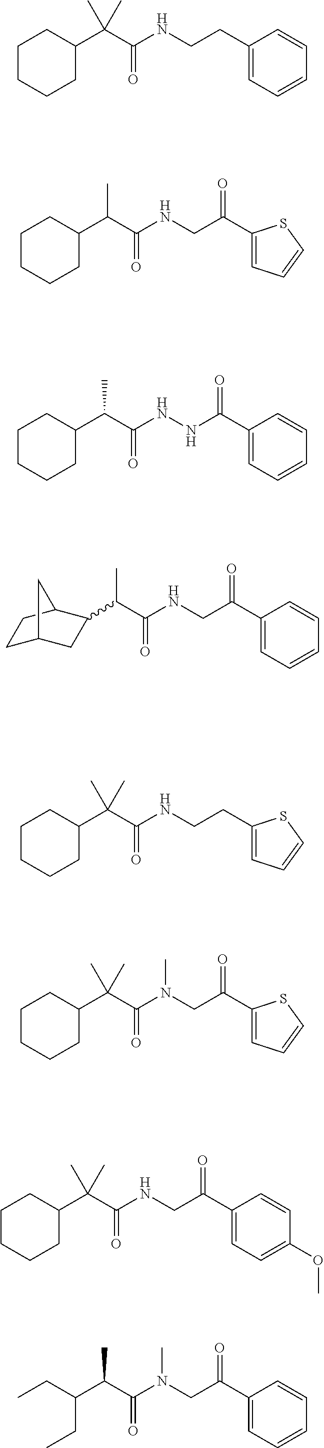

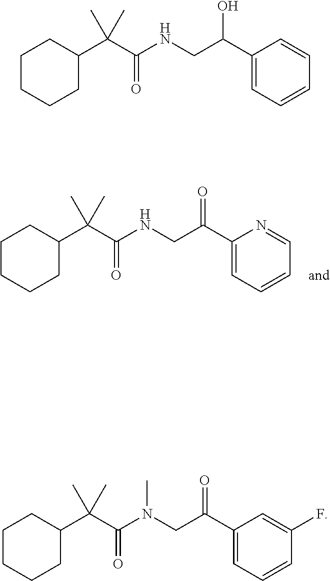 [SMILES: CC(C)(C(=O)NCC(O)c1ccccc1)C1CCCCC1 ; CC(C)(C(=O)NCC(=O)c1ccccn1)C1CCCCC1 ; "and" ; CN(CC(=O)c1cccc(F)c1)C(=O)C(C)(C)C1CCCCC1]

In some embodiments, the topical composition can be in the form of a solid, semi-solid, plaster, solution, suspension, lotion, cream, foam, gel, paste, poultice, emulsion, or a combination thereof.

In some embodiments, a method of treating insect bites, insect stings, allergenic effects, burns, scrapes, cuts, abrasions, psoriasis, dandruff, pruritus, itching, nasal complaints, sore throats, upper respiratory ailments, acne, athlete's foot, or skin irritation as result of contact with poison ivy, poison oak, or poison sumac is provided comprising applying the a composition disclosed herein to a subject in need thereof.

In some embodiments, the topical composition can be selected from a poultice for the treatment of burns, a topical cough suppressant, an anti-itch cream, an antibiotic ointment, an after-sun gel, or an after-sun lotion.

In some embodiments, the topical composition can be a personal care product selected from the group consisting of shaving products, deodorants, odorants, insect repellants, facial care products, body care products, cosmetics, soap products, and lip products.

In some embodiments, the topical composition can be selected from the group consisting of a shaving cream, a shaving lotion, and after-shave lotion, a roll-on deodorant, a spray deodorant, an air freshener, a room deodorizer, a perfume, a cologne, a hand-soap, a facial soap, a lipstick, a lip balm, a lip gloss, a body lotion, and a shower gel.

In some embodiments, a method for enhancing the massage therapy treatment is provided comprising applying a topical composition disclosed herein to a subject in need thereof. In some embodiments, the topical composition can be an aphrodisiac.

The above-disclosed compounds, or a salt or solvate thereof, can be used as modulators, e.g., agonists, of the TRPM8 receptor in personal products for modulating, e.g., inducing, chemesthetic sensations, particularly the cold, cool, or tingling sensations. These compounds are also important to the flavorings and fragrance industry because they can increase or induce/generate a cooling or cold sensation which is often associated with freshness and cleanliness. As modulators of the TRPM8 receptor, the above-disclosed compounds also have repellent effect on insects, therapeutic effect in antitumor treatments (e.g., an influencing of prostate tumors), activity in the treatment of inflammatory pain/hyperalgesia, and efficacy (as TRPM8 antagonists) in the treatment of bladder syndrome or overactive bladder.

In one embodiment of the present invention, the composition comprises a chemesthetic sensation modulating amount of the present compound. In another embodiment of the present invention, the composition comprises a chemesthetic sensation inducing amount of the present compound. In some embodiments, the chemesthetic sensation is a cold or cooling sensation. In one embodiment of the composition, the present compound is in a concentration ranging from about 0.0001 ppm to 100,000 ppm. In another embodiment of the composition, the present compound is in a concentration ranging from about 0.001 ppm to 10,000 ppm. In another embodiment of the composition, the present compound is in a concentration ranging from about 0.01 ppm to 1,000 ppm. In another embodiment of the composition, the present compound is in a concentration ranging from about 0.1 ppm to 500 ppm. In another embodiment of the composition, the present compound is in a concentration ranging from about 1 ppm to 500 ppm. In another embodiment of the composition, the present compound is in a concentration ranging from about 10 ppm to 500 ppm. In another embodiment of the composition, the present compound is in a concentration ranging from about 1 ppm to 400 ppm.

The present topical compositions typically comprise one or more compounds of Formula (I), Formula (IIa), Formula (IIb), and/or Formula (III). The topical composition can be a “pharmaceutical composition” or a composition intended for “personal care.” By “pharmaceutical composition”, it is meant a composition which is used to treat the symptoms and/or root causes of a disease or ailment. In one embodiment, the pharmaceutical composition comprises one or more compounds of the present invention and at least one pharmaceutically acceptable carrier. The pharmaceutical composition category includes both the prescription medications and the over-the-counter medications. The present compound may or may not be the therapeutically active ingredient in the pharmaceutical composition. In general, over the counter (OTC) product and oral hygiene product generally refer to product for household and/or personal use which may be sold without a prescription and/or without a visit to a medical professional. Examples of the OTC products include, but are not limited to topical analgesics and/or anesthetic; cough, cold and allergy remedies; antihistamines and/or allergy remedies; and combinations thereof. Topical analgesics and/or anesthetic include any topical creams/ointments/gels used to alleviate superficial or deep-seated aches and pains, e.g. muscle pain; teething gel; sports creams; patches with analgesic ingredient; and combinations thereof. In some embodiments, the pharmaceutical composition is hemorrhoid product. Cough, cold and allergy remedies include, but are not limited to decongestants, cough remedies, pharyngeal preparations, medicated confectionery, antihistamines and child-specific cough, cold and allergy remedies; and combination products. Antihistamines and/or allergy remedies include, but are not limited to any systemic treatments for hay fever, nasal allergies, insect bites and stings. In some embodiments, the topical pharmaceutical composition can be a sinus relief product or a nasal spray. Examples of oral hygiene product include, but are not limited to mouth cleaning strips, toothpaste, toothbrushes, mouthwashes/dental rinses, denture care, mouth fresheners, breath freshening sprays, breath freshening drops, breath freshening concentrates, chewing gum, mouth moisturizers, at-home teeth whiteners and dental floss. The topical pharmaceutical composition can further be in the form of a first aid product. For example, the topical pharmaceutical composition can be applied as an ointment or on a bandage, band aid, or in a spray. In some embodiments, the pharmaceutical can nausea relief products. For example, the composition can be a topical nausea relief product or a sea band. In some embodiments, the pharmaceutical composition can be an ingestible composition. For example, the composition can be an ingestible nausea relief composition, an antacid, or a heartburn relief composition. 100721 In some embodiments, the topical pharmaceutical composition can be a composition for the treatment and alleviation of pain in a subject. Topical compositions for providing such pain relief can comprise one or more compounds of Formula (I), Formula (IIa), Formula (IIb), and/or Formula (III). The topical composition for the relief of pain can further comprise an additional active ingredient. In some embodiments the additional active ingredient can be an anti-inflammatory agent. Topical compositions comprising a cooling agent and applied in the relief of pain are well known in the art. For example, several such compositions are described in U.S. 2011/0135627 and U.S. Pat. No. 8,105,624, which are incorporated herein by reference in their entirety.

In some embodiments, the topical pharmaceutical composition can be applied in the treatment of allergenic effects. In some embodiments the topical composition comprising one or more compounds of Formula (I), Formula (IIa), Formula (IIb), and/or Formula (III), can further comprise an antibacterial agent, an anti-inflammatory agent, and or a corticosteroid. Exemplary topical compositions used in the treatment of allergenic effects and comprising menthol and/or camphor as a cooling agent are described in U.S. 2010/0104547, which is incorporated herein by reference in its entirety. In some embodiments, the topical composition can be used in the treatment of insect bites or insect stings. In some embodiments, the topical composition can be used in the treatment of psoriasis or eczema. In some embodiments, the topical composition can be a nasal decongestant.

In some embodiments, the topical pharmaceutical composition disclosed herein can be an anti-dandruff or an anti-itch composition. The anti-dandruff and/or anti-itch composition can take several forms including, but not limited to, shampoos, soaps, ointments, eye drops and creams. The anti-dandruff composition may further comprise an anti-dandruff agent such as zinc salt of 1-hydroxy-2-pyridine thione. The anti-itch composition may further comprise an anti-itch agent such as beta-methasone valerate. Exemplary anti-itch and anti-dandruff topical compositions comprising a cooling agent are described in U.S. 2003/0161802, which is incorporated herein by reference in its entirety. Exemplary topical compositions in the form of eye drops comprising a cooling agent are described in EP 2014333 A1, which is incorporated herein by reference in its entirety.

In some embodiments, the topical pharmaceutical composition disclosed herein can be an antibacterial or an antimicrobial composition. The topical antibacterial and or antimicrobial composition disclosed herein may further comprise an antibiotic, antifungal, or anti-viral agent. For example, the topical formulation may comprise one or more compounds of Formula (I), Formula (IIa), Formula (IIb), and/or Formula (III) and a quaternary ammonium compound such as benzethonium chloride. Exemplary antibacterial and antimicrobial topical compositions comprising menthyl lactate as a cooling agent are described in U.S. Pat. No. 8,951,582, which is incorporated herein by reference in its entirety. In some embodiments, the antimicrobial composition can be applied in the treatment of athlete's foot.

In some embodiments, the topical pharmaceutical composition disclosed herein can be a composition for treatment of skin irritation as a result of contact with poison ivy, poison oak, or poison sumac. For example, the topical pharmaceutical composition can be a composition for removing urushiol, the primary irritant contracted from poision ivy, poision oak, or poison sumac. In some embodiments, the one or more compounds of Formula (Ia), Formula (IIa), Formula (IIb), and/or Formula (III) provide a soothing effect to a subject having contracted poision ivy, poison oak, or poison sumac. Exemplary topical compositions for such treatment comprising one or more cooling agents are described in U.S. Pat. No. 7,858,570, which is incorporated herein by reference in its entirety.

In some embodiments, the topical pharmaceutical composition disclosed herein can be a composition for the treatment of upper respiratory ailments. VapoRub®, sold by Vicks corporation, is an example of a topical formulation for the treatment of upper respiratory ailments, including the common cold, cough, and bronchitis, the active ingredients of which are menthol and camphor. One of skill in the art, would recognize that a compound of Formula (Ia), Formula (IIa), Formula (IIb), and/or Formula (III) would likewise be suitable in a topical composition for such treatment. Other exemplary topical compositions which have employed cooling agents in the treatment of upper respiratory ailments are described in U.S. Pat. No. 6,196,348 and WO 200205802 A1, which are incorporated herein by reference in their entirety.

In some embodiments, the topical pharmaceutical composition disclosed herein can be a composition for the treatment of acne. Cooling agents such as camphor, menthol, and menthyl lactate have been commonly used to aid in the treatment of acne. One of skill in the art would immediately recognize that a compound of Formula (Ia), Formula (IIa), Formula (IIb), and/or Formula (III) would be suitable for use as a cooling agent in the treatment of acne.

In some embodiments, the topical composition disclosed herein can be a composition for the treatment of burns, cuts, scrapes, and abrasions. In some embodiments, the topical composition is used to treat sunburns. For example, the topical composition disclosed herein can be a sun-tanning product, a sunscreen product, an after-sun lotion, an ointment, a salve, a balm, and the like.

As used herein, a “personal care composition” refers to a composition to be directly applied to the skin, mucosal, or other surface area of the body. Examples of personal care composition include, but are not limited to, an oral care composition, such as toothpaste, chewing gum, breath refresher, dentifrices, and mouthwashes; a skincare or haircare composition, such as sunscreen cream, sunburn lotions, shaving cream, plasters, shampoos, conditioners, face cleaners, facial washes, soaps, bath oils or bath foam, antiperspirants, and deodorant; a cosmetic composition, such as moisturizer, lip balms, cosmetic balms, foundation, etc.; an insect repellent composition; or an insecticide composition.

In some embodiments, the topical composition disclosed herein can be a fragrance product. As used herein, the term “fragrance product” or “odorant” relates to a product which provides an enhanced aroma. Examples of fragrance products include, but are not limited to, air fresheners, room deodorizers, perfume, eau de perfume, eau de toilet, and eau de cologne. In other embodiments the topical composition can be an antiperspirant or deodorant.

In some embodiments, the topical composition disclosed herein can be a skincare product. Examples of skincare products include, but are not limited to face washing creams, scar smoothing products, skin irritation soothing products (oils, sprays, salves, lotions, ointments, or gels), facial cleansing wipes, body cleansing wipes, anti-aging facial and/or eye creams, pore cleansing strips, pore cleansing oils, pore cleansing ointments, hair removal products (wax, wax strips, roll-ons, creams, gels, lotions, etc.), varnishing creams, cleansing creams, cold creams, massage creams, milky lotions, skin toning lotion, cosmetic solution, packs, makeup remover, and the like; as makeup cosmetics, foundations, face powders, pressed powders, talcum powders, lip sticks, lip creams, cheek powders, eyeliners, mascara, eye shadows, eyebrow pencils, eye packs, nail enamels, nail enamel removers, and the like;

In some embodiments, the topical composition disclosed herein can be a hair care product, Examples of hair care products include, but are not limited to hair care cosmetics, pomades, brillantine, setting lotions, hair sticks, hair solids, hair oils, hair treatments, hair creams, hair tonics, hair liquids, hair sprays, moose, hair gel, bandlin, hair restorers, hair dyes, and the like; further examples include shampoos, permanent wave lotions, medicated shampoos, rinses, hair conditioners, hair treatments, hair packs, and the like.

In some embodiments, the topical composition disclosed herein can be a body care or bathing composition. For example, the topical composition can be a shaving cream, a shaving gel, a shaving lotion, an after-shave lotion, an after-shave gel, a medicated soap, sanitizing gel, a hand sanitizer, sanitizing wipes, cool nasal tissues (tissues that have lotion on them), sports cooling towels, cooling fabrics, sport shirts, eye pillows, sheets, cooling pillows, hats, diapers, adult diapers, potty-training pants and pull-ups, talcum powder, baby powder, slimming gels, a bath soap, a face soap, a hand soap, exfoliating washes, hand scrub, foot scrub, shower mists, shower sprays, a body washing soap, a body washing gel, a bath salt, a bath tablet, a bath foam, a bubble-bath concentrate, a bath oil, a bath perfume, a bath capsule, a milk bath, a bath gel, or a bath cube.

In some embodiments, the topical composition disclosed herein can be a composition used as an insect repellant. JP 11171703 A, which is incorporated herein by reference in its entirety, describes a pest repellant which contains menthol.

In some embodiments, the topical composition disclosed herein can be a composition for the enhancement of massage therapy treatment. For example, the topical composition can be in the form of a massage oil, massage cream, massage lotion, or massage gel that comprises one or more compounds of Formula (I), Formula (IIa), Formula (IIb), and/or Formula (III).

In some embodiments the topical composition is an aphrodisiac composition comprising one or more compounds of Formula (I), Formula (IIa), Formula (IIb), and/or Formula (III). By “aphrodisiac” is meant a composition that improves the sexual desire and sexual satisfaction of a subject. Examples of topical aphrodisiac compositions in the form of a cream, lotion, or gel and comprising menthol or peppermint oil have been previously reported in WO 03/047610, which is incorporated herein by reference in its entirety.

### Combinations

The chemestetic effect of the compounds disclosed herein can be enhanced through the combination of one or more of the presently disclosed compounds with one or more natural or synthetic cooling agents. Through such a combination, a synergistic effect can be achieved whereby the chemestetic effect of the combination is greater than the sum of chemestetic effects of each individual agent used in the combination. A person of ordinary skill in the art would understand that the topical compositions described herein can comprise any of the compounds disclosed herein alone or in combination.

Examples of natural and synthetic cooling agents are well-known in the art. Several suitable cooling agents useful for combination with the compounds disclosed herein are described in U.S. 2013/0324557, WO 2014/130582, U.S. Pat. No. 7,923,585, U.S. 2008/0319055, U.S. Pat. No. 7,893,110, U.S. 2009/0105237, U.S. 2009/0098066, U.S. 2010/0035938, U.S. Pat. No. 8,263,046, U.S. Pat. No. 7,959,958, U.S. 2008/0300314, U.S. 2009/0312384, U.S. Pat. No. 8,309,598, U.S. Pat. No. 7,935,848, U.S. 2010/0297038, U.S. Pat. No. 8,377,422, U.S. Pat. No. 8,664,261, U.S. 2011/0091531, U.S. 2013/0323388, U.S. 2014/0341821, U.S. 2010/0086498, U.S. 2014/0186272, U.S. Pat. No. 6,884,906, U.S. 2011/0070329, U.S. Pat. No. 8,575,349, U.S. Pat. No. 5,725,865, U.S. Pat. No. 5,843,466, WO 2011/147455, U.S. Pat. No. 8,007,771, WO 2004/037764, U.S. Pat. No. 6,627,233, WO 2011/159935, U.S. Pat. No. 7,767,243, U.S. Pat. No. 7,662,576, U.S. Pat. No. 5,372,824, U.S. Pat. No. 5,009,893, U.S. Pat. No. 5,698,181, U.S. Pat. No. 7,189,760, U.S. Pat. No. 7,030,273, WO 02/091849, U.S. Pat. No. 5,286,500, U.S. Pat. No. 3,488,419, U.S. Pat. No. 6,515,188, U.S. Pat. No. 6,407,293, U.S. Pat. No. 4,459,425, U.S. Pat. No. 3,419,543, U.S. 2006/0210482, U.S. Pat. No. 6,328,982, U.S. Pat. No. 7,025,999, EP 1332772, U.S. Pat. No. 4,157,384, WO 2014/090293, U.S. 2008/0175800, U.S. Pat. No. 8,344,025, U.S. Pat. No. 8,927,605, U.S. 2011/0305657, U.S. 2013/0202543, and U.S. 2014/0335224.

In some embodiments, one or more of the compounds disclosed herein can be combined with one or more compounds selected from peppermint oil, menthol, menthone, eucalyptol, borneol, camphor, 4-terpinol, Freskomenthe (2-(1-methylpropyl)-cyclohexanone), neomenthol, isomenthol, neoisomenthol, monomethyl succinate, dimethyl succinate, ormenthyl acetate, methyl acetate, menthol ethylene glycol carbonate (marketed as Frescolat® MGC), menthol propylene clycol carbonate (marketed as Frescolat® MPC), menthone glycerol ketal (marketed as Frescolat® MGA), menthyl lactate (marketed as Frescolat® ML), 3-(1-Menthoxy) propane-1,2-diol (MPD), 3-(1-menthoxy)-2-methylpropane-1,2-diol, 3-(1-metnhoxy)ethanol (Coolact® 5), 3-(1-menthoxy)propan-1-ol, 3-(1-metnhoxy)butan-1-ol, isopulegol (Coolact® P), Questice® (menthyl pyrrolidin-2-one 5-carboxylate), WS-3 (N-ethyl-p-menthane-3-carboxamide), WS-23 (2-isopropyl-N,2,3-trimethylbutyramide), WS-14 [N-([ethoxycarbonyl]methyl)-p-menthane-3-carboxamide], WS-5 [ethyl 3-(p-menthane-3-carboxamido)acetate], N,N-dimethyl menthyl succinamide, N-(2-ethoxyethyl)-2-isopropyl-2,3-dimethylbutanamide, N-(1-isopropyl-1,2-dimethylpropyl)-1,3-benzodioxole-5-carboxamide, N-benzo[1,3]dioxol-5-yl-3-p-menthanecarboxamide, N-benzooxazol-4-yl-3-p-menthanecarboxamide, HASE-1 (1R,4S,5R)—N-(2-ethoxyethyl)-2-isopropyl-5-methylcyclohexane-1-carboxamide), N—(R)-2-oxotetrahydrofuran-3-yl-(1R,2S,5R)-p-menthane-3-carboxamide (D-HSL), L-phenylephrine p-menthane carboxamide (CPS-195), WS-12 (1R,2S,5R)—N-(4-Methoxyphenyl)-p-menthanecarboxamide, WS-27 (N-Ethyl-2,2-diisopropylbutanamide), N-Cyclopropyl-5-methyl-2-isopropylcyclohexanecarboxamide, WS-116 (N-(1,1-Dimethyl-2-hydroxyethyl)-2,2-diethylbutanamide), Evercool™ 190 (G-190, (1R,2S,5R)—N-(2-(pyridin-2-yl)ethyl)menthylcarboxamide), Ultracool 7 (menthyl acetoacetate), Evercool™ 180 ((1R,2S,5R)—N-(4-(cyanomethyl)phenyl)menthylcarboxamide), menthyl glutarate, PMD 38 (Coolact® 38, trans-p-menthane-3,8-diol), Freshone® (menthone (S)-lactic acid ketal, (−)-Cubebol ((1R,4S,5R,6R,7S,10R)-7-isopropyl-4, 10-dimethyl-tricyclo[4.4.0.0(1,5)]decan-4-ol), N-(2-Hydroxyethyl)-2,3-dimethyl-2-isopropylbutanamide, Di-(−)-menthyl glutarate, (1R,2S,5R)—N-(4-(carbamoylmethyl)phenyl)-menthylcarboxamide, (1R,2S,5R)-2-[2-(2-isopropyl-5-methyl-cyclohexyloxy)ethoxy]-ethanol, (1R,2R,4R)-1-(2-Hydroxy-4-methylcyclohexyl)ethanone, 2-(p-tolyloxy)-N-(1H-pyrazol-5-yl)-N-((thiophen-2-yl)methyl)acetamide, N-(1,1-Dimethyl-2-hydroxyethyl)2,2-diethylbutanamide, 2,2,5,6,6-pentamethyl-2,3,6,6a-tetrahydropentalen-3a(1H)-ol, 5-(2-hydroxy-2-methylpropyl)-3,4,4-trimethylcyclopent-2-en-1-one, Icilin (AG-3-5, 1-[2-hydroxyphenyl]-4-[2-nitrophenyl-]-1,2,3,6-tetrahydropyrimidine-2-one),

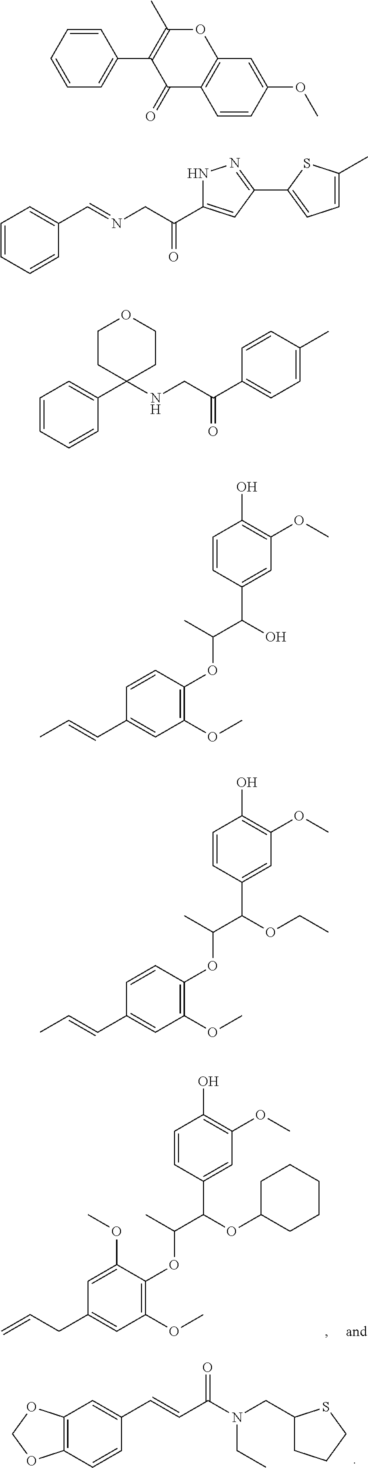

### Carriers

In one embodiment, the topical composition disclosed herein comprises i) compounds as disclosed and described herein, individually or in combination; ii) a carrier; and iii) optionally at least one adjuvant. The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, which is incorporated herein by reference in its entirety.

Some examples of substances, which can serve as pharmaceutically-acceptable carriers or components thereof, are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers, such as the TWEENS; wetting agents, such sodium lauryl sulfate; coloring agents; flavoring agents; tableting agents, stabilizers; antioxidants; preservatives; pyrogen-free water; isotonic saline; and phosphate buffer solutions.

The term “adjuvant” denotes an additive which supplements, stabilizes, maintains, or enhances the intended function or effectiveness of the active ingredient, such as the compound of the present invention. In one embodiment, the at least one adjuvant comprises one or more topically acceptable materials that may be used to preserve or alter the properties of the topical composition disclosed herein. These materials include, but are not limited to, materials having anti-acne, anti-ageing, anti-wrinkle, antifungal, anti-inflammatory, antimicrobial, antioxidant, antiperspirant, antidandruff, anti-dermatitis, antipruritic, anti-emetic, anti-dry skin, anti-psoriatic, anti-seborrhea, anti-asthmatic, astringents, bronchodilators, biocides, chemical exfoliants, cleansers, colorants, corticosteroids, deodorants, depigmenting, depilating, emollients, epilating, analgesics, hair conditioners, hormones, humectants, light-interacting, luster-imparting, make-up removers, pH adjusters, powders, rheological modifiers, shine-imparting, skin bleaching, skin conditioning, skin protecting, tanning, UV screening vitamins, and/or wound-healing properties.

In one embodiment, the present topical formulation can be in a form selected from the group consisting of liquid, including solution and suspension, solid, foamy material, emulsion, paste, gel, cream, and a combination thereof, such as a liquid containing a certain amount of solid contents. In one embodiment, the flavoring concentrate formulation is in form of a liquid including aqueous-based and nonaqueous-based.

In some embodiments, the topical composition is an ophthalmic composition. A liquid composition, which is formulated for topical ophthalmic use, is formulated such that it can be administered topically to the eye. The comfort may be maximized as much as possible, although sometimes formulation considerations (e.g. compound stability) may necessitate less than optimal comfort. In the case that comfort cannot be maximized, the liquid may be formulated such that the liquid is tolerable to the patient for topical ophthalmic use. Additionally, an ophthalmically acceptable liquid may either be packaged for single use, or contain a preservative to prevent contamination over multiple uses.

For ophthalmic application, solutions or medicaments are often prepared using a physiological saline solution as a major vehicle. Ophthalmic solutions may preferably be maintained at a comfortable pH with an appropriate buffer system. The formulations may also contain conventional, biologically acceptable preservatives, stabilizers and surfactants.

Preservatives that may be used in the topical compositions disclosed herein include, but are not limited to, benzalkonium chloride, PHMB, chlorobutanol, thimerosal, phenylmercuric, acetate and phenylmercuric nitrate. A useful surfactant is, for example, Tween 80. Likewise, various useful vehicles may be used in the ophthalmic preparations disclosed herein. These vehicles include, but are not limited to, polyvinyl alcohol, povidone, hydroxypropyl methyl cellulose, poloxamers, carboxymethyl cellulose, hydroxyethyl cellulose and purified water.

Tonicity adjustors may be added as needed or convenient. They include, but are not limited to, salts, particularly sodium chloride, potassium chloride, mannitol and glycerin, or any other suitable ophthalmically acceptable tonicity adjustor.

Various buffers and means for adjusting pH may be used so long as the resulting preparation is ophthalmically acceptable. For many compositions, the pH will be between 4 and 9. Accordingly, buffers include acetate buffers, citrate buffers, phosphate buffers and borate buffers. Acids or bases may be used to adjust the pH of these formulations as needed.

Ophthalmically acceptable antioxidants include, but are not limited to, sodium metabisulfite, sodium thiosulfate, acetylcysteine, butylated hydroxyanisole and butylated hydroxytoluene.

Other excipient components, which may be included in the ophthalmic preparations, are chelating agents. A useful chelating agent is edetate disodium, although other chelating agents may also be used in place or in conjunction with it.

Topical formulations may generally be comprised of a pharmaceutical carrier, co-solvent, emulsifier, penetration enhancer, preservative system, and emollient.

In some embodiments, the topical composition disclosed herein may comprise an aqueous component. For example, the composition can be a cream, lotion, ointment, conditioning shampoo, moisturizing hand soap, etc. In some embodiments, the topical composition disclosed herein can comprise about 35% (w/w) to about 90% (w/w), about 40% (w/w) to about 85% (w/w), about 45% (w/w) to about 80% (w/w), about 50% (w/w) to about 75% (w/w), about 55% (w/w) to about 70% (w/w), about 60% (w/w) to about 65% (w/w), or about 62% (w/w) of water. In some embodiments, the topical composition disclosed herein can comprise at least 50% (w/w), 55% (w/w), 60% (w/w), 65% (w/w), 70% (w/w), 75% (w/w), 80% (w/w), or 85% (w/w) of water. In some embodiments, the topical composition disclosed herein can comprise up to 50% (w/w), 55% (w/w), 60% (w/w), 65% (w/w), 70% (w/w), 75% (w/w), 80% (w/w), or 85% (w/w) of water. In some embodiments, the topical composition disclosed herein can comprise 50% (w/w), 55% (w/w), 60% (w/w), 65% (w/w), 70% (w/w), 75% (w/w), 80% (w/w), or 85% (w/w) of water or a range defined by any two of the preceding values.

Some embodiments provide a topical composition including skin penetration enhancers.

Examples of suitable skin penetration enhancers include alcohols, fatty acids, fatty acid esters, polyols, sulphoxides, glyceryl monooleate, lauryl lactate, Dodecyl-2-(N,N-dimethyl)-amino propionate (DDAIP), N-(4-bromobenzoyl)-S,S-dimethyliminosulfurane, NexACT enhancers, 2-nonyl-1,3-dioxolane (SEPA®), 1-dodecylazacycloheptan-2-one (Azone®), pyrrolidones, essential oil, terpenes, terpenoids, oxazolidinones, urea and the like.

Examples of suitable fatty acids include, but are not limited to, valeric acid, heptanoic acid, pelagonic acid, caproic acid, capric acid, lauric acid, myristic acid, stearic acid, oleic acid, and caprylic acid; and branched fatty acids, such as isovaleric acid, neopentanoic acid, neoheptanoic acid, neononanoic acid, trimethyl hexanoic acid, neodecanoic acid, and isostearic acid.

Examples of suitable fatty acid esters include but are not limited to, isopropyl n-butyrate, isopropyl n-hexanoate, isopropyl n-decanoate, isopropyl myristate, isopropyl palmitate, and octyldodecyl myristate; alkyl fatty acid esters such as ethyl acetate, butyl acetate, methyl acetate, methylvalerate, methylpropionate, diethyl sebacate, and ethyl oleate; and diisopropyl adipate and dimethyl isosorbide.

Suitable skin penetration enhancers are known in the art and include, but are not limited to, monoglycerides, polyglycosylated glycerides, glyceryl monoethyl ether, polysorbates, beta-cyclodextrin, cyclopentadecalactone, alkyl-2-(N,N-disubstituted amino)-alkanoate ester, 2-(n-nonyl)-1,3-dioxolane, isopropyl myristate, terpinol, menthol, cineol, monoolein, sodium oleate, oleyl oleate, laurylcapram, bisabolol, capsaicin, and capsicum. Other examples of suitable skin penetration enhancers and a description of their mechanism of action may be found in Goodman and Barry, “Percutaneous Absorption,” in Mechanisms-Methodology-Drug Delivery, 2nd Edition, Bronaugh and Maibach, eds., 1989, pp. 567-593, Marcel Dekker, Inc., NY, which is incorporated herein by reference in its entirety.

In some embodiments, the skin penetration enhancer can be selected from the group consisting of n-octanol, D-limonene, oleic acid, cineol, isopropyl myristate, monooleate, monoolein, sodium oleate, oleyl oleate, laurylcapram, sodium lauryl sulfate, bisabolol, lauric acid, myristic acid, isopropyl palmitate, diisopropyl adipate, dimethyl isosorbide, propylene glycol, butylene glycol, polyethylene glycol, dipropylene glycol, ethoxydiglycol, and pentylene glycol or combinations thereof. In a typical embodiment, the skin penetration enhancer can be selected from the group consisting of oleic acid, laurocapram, sodium lauryl sulphate, bisabolol, lauric acid, myristic acid, isopropyl myristate, isopropyl palmitate, diisopropyl adipate, dimethyl isosorbide, propylene glycol, butylene glycol, polyethylene glycol, dipropylene glycol, ethoxydiglycol, and pentylene glycol, or combinations thereof.

In some embodiments, the topical composition disclosed herein can comprise at least 5% (w/w), 6% (w/w), 7% (w/w), 8% (w/w), 9% (w/w), 10% (w/w), 11% (w/w), or 12% (w/w) of a skin penetration enhancer. In some embodiments, the topical composition disclosed herein can comprise 5% (w/w), 6% (w/w), 7% (w/w), 8% (w/w), 9% (w/w), 10% (w/w), 11% (w/w), 12% (w/w), 13% (w/w), 14% (w/w), 15% (w/w), 16% (w/w), 17% (w/w), 18% (w/w), 19% (w/w), 20% (w/w), 21% (w/w), 22% (w/w), 23% (w/w), 24% (w/w), 25% (w/w), 26% (w/w), 27% (w/w), 28% (w/w), 29% (w/w), or 30% (w/w)) of a skin penetration enhancer or a range defined by any two of the preceding values. In a typical embodiment, the skin penetration enhancer can be ethoxydiglycol.

In some embodiments, the composition can include an emollient. In some embodiments, the emollient can be alkyl dimethicones, alkyl methicones, alkyldimethicone copolyols, phenyl silicones, alkyl trimethylsilanes, dimethicone, dimethicone crosspolymers, cyclomethicone, lanolin and its derivatives, fatty esters, glycerol esters and derivatives, propylene glycol esters and derivatives, alkoxylated carboxylic acids, alkoxylated alcohols, fatty alcohols, and combinations thereof. In some embodiments, the emollient can be selected from the group consisting of cetyl palmitate, stearyl palmitate, cetyl stearate, isopropyl laurate, isopropyl myristate, and isopropyl palmitate, or combinations thereof. In some embodiments, the emollient can be selected from the group consisting of octyldodecanol, lauryl, myristyl, cetyl, stearyl, and behenyl alcohol, or combinations thereof. In some embodiments, the emollient can be selected from the group consisting of eucalyptol, ceteraryl glucoside, dimethyl isosorbic polyglyceryl-3 cetyl ether, polyglyceryl-3 decyltetradecanol, propylene glycol and myristyl ether, or combinations thereof.

In some embodiments, the topical composition disclosed herein can comprise at least 1% (w/w), 2% (w/w), 3% (w/w), 4% (w/w), 5% (w/w), 6% (w/w), 7% (w/w), 8% (w/w), 9% (w/w), 10% (w/w), 11% (w/w), or 12% (w/w) of an emollient. In some embodiments, the topical composition disclosed herein can comprise 1% (w/w), 2% (w/w), 3% (w/w), 4% (w/w), 5% (w/w), 6% (w/w), 7% (w/w), 8% (w/w), 9% (w/w), 10% (w/w), 11% (w/w), 12% (w/w), 13% (w/w), 14% (w/w), 15% (w/w), 16% (w/w), 17% (w/w), 18% (w/w), 19% (w/w), 20% (w/w), 21% (w/w), 22% (w/w), 23% (w/w), 24% (w/w), 25% (w/w), 26% (w/w), 27% (w/w), 28% (w/w), 29% (w/w), or 30% (w/w)) of an emollient or a range defined by any two of the preceding values.

In some embodiments, the topical composition disclosed herein can comprise natural fats and oils. In some embodiments, the natural fats and oils can be selected from the group consisting of citrus oil, olive oil, avocado oil, apricot oil, babassu oil, borage oil, camellia oil, canola oil, castor oil, coconut oil, corn oil, cottonseed oil, emu oil, evening primrose oil, hydrogenated cottonseed oil, hydrogenated palm kernel oil, maleated soybean oil, meadowfoam oil, palm kernel oil, peanut oil, rapeseed oil, grapeseed oil, safflower oil, sphingolipids, seed almond oil, tall oil, lauric acid, palmitic acid, stearic acid, linoleic acid, stearyl alcohol, lauryl alcohol, myristyl alcohol, behenyl alcohol, rose hip oil, calendula oil, chamomile oil, eucalyptus oil, juniper oil, sandlewood oil, tea tree oil, sunflower oil, and soybean oil, or combinations thereof.

In some embodiments, the topical composition disclosed herein can comprise at least 5% (w/w), 6% (w/w), 7% (w/w), 8% (w/w), 9% (w/w), 10% (w/w), 11% (w/w), or 12% (w/w) of a vegetable oil. In some embodiments, the topical composition disclosed herein can comprise 5% (w/w), 6% (w/w), 7% (w/w), 8% (w/w), 9% (w/w), 10% (w/w), 11% (w/w), 12% (w/w), 13% (w/w), 14% (w/w), 15% (w/w), 16% (w/w), 17% (w/w), 18% (w/w), 19% (w/w), 20% (w/w), 21% (w/w), 22% (w/w), 23% (w/w), 24% (w/w), 25% (w/w), 26% (w/w), 27% (w/w), 28% (w/w), 29% (w/w), or 30% (w/w)) of a vegetable oil or a range defined by any two of the preceding values. In a typical embodiment, the topical composition disclosed herein can comprise coconut oil.

In some embodiments, the topical composition disclosed herein may optionally further comprise ingredients to relieve irritation, such as anti-itch agents. In some embodiments, the anti-itch agents may be present in the topical composition disclosed herein in an amount of from about 0.1% to about 33% (w/w), more typically, from about 0.5% to about 5% (w/w). Examples of suitable anti-itch agents are listed below, as well as the preferred concentration for each agent, given in percent by weight of the composition: lauromacrogols, benzocaine (about 5% to about 20%), butamben picrate (about 1%), dibucaine (about 0.25% to about 1%), dibucaine hydrochloric acid (0.25% to about 1%), dimethisoquin hydrochloric acid (about 0.3% to about 0.5%), dyclonine hydrochloric acid (about 0.5% to about 1%), lidocaine (about 0.5% to about 5%), lidocaine hydrochloric acid (about 0.5% to about 5%), pramoxine hydrochloric acid (about 0.5% to about 1%), tetracaine (about 1% to about 2%), tetracaine hydrochloric acid (about 1% to about 2%), benzyl alcohol (about 10% to about 33%), camphor (about 0.1% to about 3%), juniper tar (about 1% to about 5%), menthol (about 0.1% to about 1%), phenol (about 0.5% to about 1.5%), phenolate sodium (about 0.5% to about 1.5%), resorcinol (about 0.5% to about 3%), diphenhydramine hydrochloric acid (about 1% to about 2%), tripelennamine hydrochloric acid (about 0.5% to about 2%), hydrocortisone (about 0.1% to about 5%, preferably about 0.5% to about 2.5%), and combinations thereof. In some embodiments, the topical composition disclosed herein may optionally also comprise cosmetic anti-itch ingredients such as, for example, Symcalmin® (Symrise GmbH & Co., Holzminden, Germany).

In some embodiments, the compositions may include adjunct components conventionally found in pharmaceutical compositions in their art-established fashion and at their art-established levels. For example, the compositions may comprise additional compatible pharmaceutically active materials for combination therapy, such as antimicrobials, antioxidants, anti-parasitic agents, antipruritics, antifungals, antiseptic actives, biological actives, astringents, keratolytic actives, local anaesthetics, anti-stinging agents, anti-reddening agents, skin soothing agents, and combinations thereof.

In some embodiments, the compositions may include colorants, deodorants, fragrances, perfumes, emulsifiers, anti-foaming agents, lubricants, natural moisturizing agents, skin conditioning agents, skin protectants and skin benefit agents (e.g., aloe vera and laponite), solvents, solubilizing agents, suspending agents, wetting agents, humectants, preservatives, propellants, dyes and/or pigments, and combinations thereof. In some embodiments, the compositions may have a particularly pleasant fragrance. In some embodiments, the compositions may have a particularly pleasant texture. In some embodiments, the compositions may have a particularly pleasant soothing effect.

In some embodiments, the compositions may include excipients conventionally found in topical compositions.

In some embodiments, the excipients can include a viscosity-adjusting agent. In some embodiments, the viscosity adjusting agents can be selected from the group consisting of long chain alcohols, cellulose ethers, gums, magnesium aluminum silicate, silica, microcrystalline wax, beeswax, paraffin, and cetyl palmitate, homopolymers, and copolymers. In some embodiments, the long chain alcohols can be cetyl alcohol, stearyl alcohol, or cetearyl alcohol. In some embodiments, the cellulose ethers can be hydroxypropylmethylcellulose, hydroxyethylcellulose, hydroxypropylcellulose, or carboxymethylcellulose. In some embodiments, the gum can be xanthan gum or sclerotium gum. In a particular embodiment, the viscosity adjusting agents can include xanthan gum. In another embodiment, the viscosity adjusting agents is xanthan gum. In another embodiment, the viscosity adjusting agent is a polyalkylene oxide such as polyethylene glycol. In other embodiments, the viscosity adjusting agent is pullulan. In other embodiments, the viscosity adjusting agent is a polyvinyl halide, such as polyvinyl chloride.

In some embodiments, the topical composition disclosed herein can comprise at least 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), or 1.2% (w/w) of a viscosity adjusting agent. In some embodiments, the topical composition disclosed herein can comprise 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), 1.2% (w/w), 1.5% (w/w), 2% (w/w), 3% (w/w), 4% (w/w), 5% (w/w), 6% (w/w), 7% (w/w), 8% (w/w), 9% (w/w), 10% (w/w), 20% (w/w) or 30% (w/w) of a viscosity adjusting agent or a range defined by any two of the preceding values.

In some embodiments, the topical composition disclosed herein can comprise at least 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), or 1.2% (w/w) of xanthan gum. In some embodiments, the topical composition disclosed herein can comprise 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), 1.2% (w/w), 1.5% (w/w), 2% (w/w), 3% (w/w), 4% (w/w), 5% (w/w), 6% (w/w), 7% (w/w), 8% (w/w), 9% (w/w), 10% (w/w), 20% (w/w) or 30% (w/w) of xanthan gum or a range defined by any two of the preceding values.

In some embodiments, the excipients can include a topical pharmaceutical and cosmetically-acceptable emollient. As used herein, “emollients” refer to materials used for the prevention or relief of dryness, as well as for the protection of the skin. Sagarin, Cosmetics, Science and Technology, 2nd Edition, Vol. 1, pp. 32-43 (1972), which is incorporated herein by reference in its entirety, contains numerous examples of suitable materials for use as emollients. Examples of classes of useful emollients include, but are not limited to, hydrocarbon oils and waxes such as mineral oil, petrolatum, paraffin, ceresin, ozokerite, microcrystalline wax, polyethylene, and perhydrosqualene; silicone oil, such as dimethyl polysiloxanes, methylphenyl polysiloxanes, and water-soluble and alcohol-soluble silicone glycol copolymers. Other suitable emollients include triglyceride esters such as vegetable and animal fats and oils including castor oil, safflower oil, cotton seed oil, corn oil, olive oil, cod liver oil, almond oil, avocado oil, palm oil, sesame oil, and soybean oil; acetoglyceride esters, such as acetylated monoglycerides; ethoxylated glycerides, such as ethoxylated glycerylmonostearate; alkyl esters of fatty acids including methyl, isopropyl, and butyl esters of fatty acids, alkyl esters including hexyl laurate, isohexyl laurate, iso-hexyl palmitate, isopropyl palmitate, decyl oleate, isodecyl oleate, hexadecyl stearate, decyl stearate, isopropyl isostearate, diisopropyl adipate, dissohexyl adipate, di-hexyldecyl adipate, diisopropyl sebacate, lauryl lactate, myristyl lactate, and cetyl lactate; and alkenyl esters of fatty acids such as oleyl myristate, oleyl stearate, and oleyl oleate. Other suitable classes of emollients include fatty acids such as pelargonic, lauric, myristic, palmitic, stearic, isostearic, hydroxystearic, oleic, linoleic, ricinoleic, arachidic, behenic, and erucic acids; fatty alcohols such as lauryl, myristyl, cetyl, hexadecyl, stearyl, isostearyl, hydroxystearyl, oleyl, ricinoleyl, behenyl, and erucyl alcohols, as well as 2-octyl dodecanol; fatty alcohol ethers; ethoxylated fatty alcohols; ether-esters such as fatty acid esters of ethoxylated fatty alcohols; lanolin and derivatives including lanolin oil, lanolin wax, lanolin alcohols, lanolin fatty acids, isopropyllanolate, ethoxylated lanolin, ethoxylated lanolin alcohols, ethoxolated cholesterol, propoxylated lanolin alcohols, acetylated lanolin, acetylated lanolin alcohols, lanolin alcohols linoleate, lanolin alcohols recinoleate, acetate of lanolin alcohols recinoleate, acetate of lanolin alcohols recinoleate, acetate of ethoxylated alcohols esters, hydrogenolysis of lanolin, ethoxylated hydrogenated lanolin, ethoxylated sorbitol lanolin, and liquid and semisolid lanolin absorption bases are illustrative of emollients derived from lanolin; polyhydric alcohols and polyether derivatives such as propylene glycol, dipropylene glycol, polypropylene glycols, polyoxyethylene polyoxypropylene glycols, polyoxypropylene polyoxyethylene glycols, glycerol, sorbitol, ethoxylated sorbitol, hydroxypropylsorbitol, polyethylene glycols, methoxy polyethylene glycols, polyalkylene glycols and derivatives, hexylene glycol(2-methyl-2,4-pentanediol), 1,3-butylene glycol, 1,2,6-hexanetriol, 2-ethyl,3-hexanediol, and polyoxypropylene derivatives of trimethylolpropane; polydydric alcohol esters such as ethylene glycol mono- and di-fatty acid esters, diethylene glycol mono- and di-fatty acid esters, polyethylene glycol, mono- and di-fatty acid esters, propylene glycol mono- and di-fatty esters, polypropylene glycol monooleate, polypropylene glycol monostearate, ethoxylatedpropylene glycol monostearate, glyceryl mono- and di-fatty acid esters, polyglycerol poly-fatty acid esters, ethoxylated glyceryl monostearate, 1,3-butylene glycolmonostearate, 1,3-butylene glycol distearate, polyoxyethylene polyol fatty acid ester, sorbitan fatty acid esters, and polyoxyethylene sorbitan fatty acid esters; wax esters such as beeswax, spermaceti, myristyl myristate and stearyl stearate; beeswax derivatives, e.g., polyoxyethylene sorbitol beeswax; vegetable waxes including carnauba and candelilla waxes; and phospholipids, such as lecithin and derivatives; sterols including, for example, cholesterol and cholesterol fatty acid esters; amides such as fatty acid amides, ethoxylated fatty acid amides and solid fatty acid alkanolamides. In some embodiments, the emollient can be selected from the group consisting of glycerol, hexanetriol, butanetriol, lactic acid, urea, pyrrolidone carboxylic acid, amino acids, guanidine, diglycerol and triglycerol. In a typical embodiment, the emollient can include glycerol.

In some embodiments, the topical composition disclosed herein can comprise at least 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), 1.2% (w/w), 1.3% (w/w), 1.4% (w/w), 1.5% (w/w), 1.6% (w/w), 1.7% (w/w), 1.8% (w/w), 1.9% (w/w), 2.0% (w/w), 2.1% (w/w), 2.2% (w/w), 2.3% (w/w), 2.4% (w/w), 2.5% (w/w), 2.6% (w/w), 2.7% (w/w), 2.8% (w/w), 2.9% (w/w), 3.0% (w/w), 3.1% (w/w), 3.2% (w/w), 3.3% (w/w), 3.4% (w/w), 3.5% (w/w), 3.6% (w/w), 3.7% (w/w), 3.8% (w/w), 3.9% (w/w), or 4.0% (w/w) of an emollient. In some embodiments, the topical composition disclosed herein can comprise 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), 1.2% (w/w), 1.3% (w/w), 1.4% (w/w), 1.5% (w/w), 1.6% (w/w), 1.7% (w/w), 1.8% (w/w), 1.9% (w/w), 2.0% (w/w), 2.1% (w/w), 2.2% (w/w), 2.3% (w/w), 2.4% (w/w), 2.5% (w/w), 2.6% (w/w), 2.7% (w/w), 2.8% (w/w), 2.9% (w/w), 3.0% (w/w), 3.1% (w/w), 3.2% (w/w), 3.3% (w/w), 3.4% (w/w), 3.5% (w/w), 3.6% (w/w), 3.7% (w/w), 3.8% (w/w), 3.9% (w/w), 4% (w/w), 5% (w/w), 6% (w/w), 7% (w/w), 8% (w/w), 9% (w/w), 10% (w/w), 20% (w/w) or 30% (w/w) of an emollient or a range defined by any two of the preceding values.

In some embodiments, the topical composition disclosed herein can comprise at least 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), 1.2% (w/w), 1.3% (w/w), 1.4% (w/w), 1.5% (w/w), 1.6% (w/w), 1.7% (w/w), 1.8% (w/w), 1.9% (w/w), 2.0% (w/w), 2.1% (w/w), 2.2% (w/w), 2.3% (w/w), 2.4% (w/w), 2.5% (w/w), 2.6% (w/w), 2.7% (w/w), 2.8% (w/w), 2.9% (w/w), 3.0% (w/w), 3.1% (w/w), 3.2% (w/w), 3.3% (w/w), 3.4% (w/w), 3.5% (w/w), 3.6% (w/w), 3.7% (w/w), 3.8% (w/w), 3.9% (w/w), or 4.0% (w/w) of glycerol. In some embodiments, the topical composition disclosed herein can comprise 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), 1.2% (w/w), 1.3% (w/w), 1.4% (w/w), 1.5% (w/w), 1.6% (w/w), 1.7% (w/w), 1.8% (w/w), 1.9% (w/w), 2.0% (w/w), 2.1% (w/w), 2.2% (w/w), 2.3% (w/w), 2.4% (w/w), 2.5% (w/w), 2.6% (w/w), 2.7% (w/w), 2.8% (w/w), 2.9% (w/w), 3.0% (w/w), 3.1% (w/w), 3.2% (w/w), 3.3% (w/w), 3.4% (w/w), 3.5% (w/w), 3.6% (w/w), 3.7% (w/w), 3.8% (w/w), 3.9% (w/w), 4% (w/w), 5% (w/w), 6% (w/w), 7% (w/w), 8% (w/w), 9% (w/w), 10% (w/w), 20% (w/w) or 30% (w/w) of glycerol or a range defined by any two of the preceding values.

In some embodiments, the excipients can include fatty acid triglycerides, namely the triglycerol esters of saturated and/or unsaturated, branched and/or unbranched alkanecarboxylic acids having a chain length of from 6 to 24 carbon atoms, in particular 6-10 carbon atoms. In a particular embodiment, the ester oil can be caprylic/capric triglyceride.

In some embodiments, the topical composition disclosed herein can comprise at least 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), or 1.2% (w/w) of a fatty acid triglyceride. In some embodiments, the topical composition disclosed herein can comprise 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), 1.2% (w/w), 1.5% (w/w), 2% (w/w), 3% (w/w), 4% (w/w), 5% (w/w), 6% (w/w), 7% (w/w), 8% (w/w), 9% (w/w), 10% (w/w), 20% (w/w) or 30% (w/w) of a fatty acid triglyceride or a range defined by any two of the preceding values.

In some embodiments, the topical composition disclosed herein can comprise at least 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), or 1.2% (w/w) of caprylic/capric triglyceride. In some embodiments, the topical composition disclosed herein can comprise 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), 1.2% (w/w), 1.5% (w/w), 2% (w/w), 3% (w/w), 4% (w/w), 5% (w/w), 6% (w/w), 7% (w/w), 8% (w/w), 9% (w/w), 10% (w/w), 20% (w/w) or 30% (w/w) of caprylic/capric triglyceride or a range defined by any two of the preceding values.

In some embodiments, the excipients can include fatty acid triglycerides, namely the triglycerol esters of saturated and/or unsaturated, branched and/or unbranched alkanecarboxylic acids having a chain length of from 6 to 24 carbon atoms, in particular 6-10 carbon atoms. In a particular embodiment, the fatty acid triglyceride can be caprylic/capric triglyceride.

In some embodiments, the excipients can include fatty acid diglycerides, namely the diglycerol esters of saturated and/or unsaturated, branched and/or unbranched alkanecarboxylic acids having a chain length of from 6 to 24 carbon atoms, in particular 6-10 carbon atoms.

In some embodiments, the topical composition disclosed herein can comprise at least 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), or 1.2% (w/w) of a fatty acid diglyceride. In some embodiments, the topical composition disclosed herein can comprise 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), 1.2% (w/w), 1.5% (w/w), 2% (w/w), 3% (w/w), 4% (w/w), 5% (w/w), 6% (w/w), 7% (w/w), 8% (w/w), 9% (w/w), 10% (w/w), 20% (w/w) or 30% (w/w) of a fatty acid diglyceride or a range defined by any two of the preceding values.

In some embodiments, the excipients can include fatty acid monoglycerides, namely the monoglycerol esters of saturated and/or unsaturated, branched and/or unbranched alkanecarboxylic acids having a chain length of from 6 to 24 carbon atoms, in particular 6-10 carbon atoms.

In some embodiments, the topical composition disclosed herein can comprise at least 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), or 1.2% (w/w) of a fatty acid monoglyceride. In some embodiments, the topical composition disclosed herein can comprise 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), 1.2% (w/w), 1.5% (w/w), 2% (w/w), 3% (w/w), 4% (w/w), 5% (w/w), 6% (w/w), 7% (w/w), 8% (w/w), 9% (w/w), 10% (w/w), 20% (w/w) or 30% (w/w) of a fatty acid monoglyceride or a range defined by any two of the preceding values.

In some embodiments, the excipients can include an emulsifier. Suitable emulsifiers are disclosed in, for example, in McCutcheon's Detergents and Emulsifiers, North American Edition, pp. 317-324 (1986), and the ICI Handbook, pp. 1673-1686, which are incorporated herein by reference in their entirety. In some embodiments, the emulsifier can include glycerol monostearate. In some embodiments, the emulsifier can include polyoxyl stearate. In some embodiments, the emulsifier can include glycerol monostearate and polyoxyl stearate. In some embodiments, the emulsifier can include PEG-6 Stearate and Glycol stearate and PEG-32 stearate. In some embodiments, the emulsifier can include glycerol monostearate, PEG-6 Stearate, Glycol stearate and PEG-32 stearate.

In some embodiments, the topical composition disclosed herein can comprise at least 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1% (w/w), 2% (w/w), 3% (w/w), 4% (w/w), 5% (w/w), 6% (w/w), 7% (w/w), 8% (w/w), 9% (w/w), 10% (w/w), 11% (w/w), 12% (w/w), 13% (w/w), 14% (w/w), 15% (w/w), 16% (w/w), 17% (w/w), 18% (w/w), 19% (w/w), 20% (w/w), 30% (w/w), or 40% (w/w) of an emulsifier. In some embodiments, the topical composition disclosed herein can comprise 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1% (w/w), 2% (w/w), 3% (w/w), 4% (w/w), 5% (w/w), 6% (w/w), 7% (w/w), 8% (w/w), 9% (w/w), 10% (w/w), 11% (w/w), 12% (w/w), 13% (w/w), 14% (w/w), 15% (w/w), 16% (w/w), 17% (w/w), 18% (w/w), 19% (w/w), 20% (w/w), 30% (w/w), or 40% (w/w) of an emulsifier or a range defined by any two of the preceding values. In some embodiments, the emulsifier can include one or more components, two or more components or three or more components.

In some embodiments, the topical composition disclosed herein can comprise at least 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1% (w/w), 2% (w/w), 3% (w/w), 4% (w/w), 5% (w/w), 6% (w/w), 7% (w/w), 8% (w/w), 9% (w/w), 10% (w/w), 11% (w/w), 12% (w/w), 13% (w/w), 14% (w/w), 15% (w/w), 16% (w/w), 17% (w/w), 18% (w/w), 19% (w/w), 20% (w/w), 30% (w/w), or 40% (w/w) of an emulsifier including glycerol monostearate, PEG-6 Stearate, Glycol stearate and PEG-32 stearate. In some embodiments, the topical composition disclosed herein can comprise 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1% (w/w), 2% (w/w), 3% (w/w), 4% (w/w), 5% (w/w), 6% (w/w), 7% (w/w), 8% (w/w), 9% (w/w), 10% (w/w), 11% (w/w), 12% (w/w), 13% (w/w), 14% (w/w), 15% (w/w), 16% (w/w), 17% (w/w), 18% (w/w), 19% (w/w), 20% (w/w), 30% (w/w), or 40% (w/w) of an emulsifier including glycerol monostearate, PEG-6 Stearate, Glycol stearate and PEG-32 stearate or a range defined by any two of the preceding values. In some embodiments, the can be a mixture of glycerol monostearate, PEG-6 Stearate, Glycol stearate and PEG-32 stearate.

In some embodiments, the excipients can include preservatives. In some embodiments, the preservatives can be selected from the group consisting of benzyl alcohol, methyl paraben, propyl paraben, DMDM hydantoin, methylchloroisothiaoline, methylisothiazolinone, imidazolidinyl urea phenoxyethanol, sodium benzoate and benzoic acid. In some embodiments, the preservatives can include phenoxyethanol, propyl paraben, and methyl paraben. In some embodiments, the preservatives can include benzalkonium chloride and/or poly(hexamethylenebiguanide) hydrochloride (PHMB).

In some embodiments, the topical composition disclosed herein can comprise at least 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), or 1.2% (w/w) of a preservative. In some embodiments, the topical composition disclosed herein can comprise 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), 1.2% (w/w), 1.5% (w/w), 2% (w/w), 3% (w/w), 4% (w/w), 5% (w/w), 6% (w/w), 7% (w/w), 8% (w/w), 9% (w/w), 10% (w/w), 20% (w/w) or 30% (w/w) of a preservative or a range defined by any two of the preceding values. In some embodiments, the preservative can include one or more components, two or more components or three or more components.

In some embodiments, the topical composition disclosed herein can comprise at least 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), or 1.2% (w/w) of a preservative including phenoxyethanol, propyl paraben, and methyl paraben. In some embodiments, the topical composition disclosed herein can comprise 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), 1.2% (w/w), 1.5% (w/w), 2% (w/w), 3% (w/w), 4% (w/w), 5% (w/w), 6% (w/w), 7% (w/w), 8% (w/w), 9% (w/w), 10% (w/w), 20% (w/w) or 30% (w/w) of a preservative including phenoxyethanol, propyl paraben, and methyl paraben or a range defined by any two of the preceding values.

In some embodiments, the topical composition may include colorants, deodorants, fragrances, perfumes, anti-foaming agents, lubricants, natural moisturizing agents, skin conditioning agents, skin protectants, skin benefit agents, solvents, solubilizing agents, suspending agents, wetting agents, humectants, propellants, dyes, pigments, and combinations thereof.

In some embodiments, the topical composition may include additional components added to enhance the odor, texture or color of the composition. For example, fragrances may be added to enhance odor. For example, emulsifiers or inert spheres may be added to enhance texture. For example, colorants may be added to enhance color.

In some embodiments, the topical composition may be applied to a body portion, such as a hand, foot, knee, elbow, and the like to treat pain and/or inflammation of the body portion. The composition may be applied by any suitable means, such as rubbing, spraying, rolling, wiping, and the like, and massaged into the body portion to be treated.

In some embodiments, the compounds as disclosed and described herein and/or topical compositions thereof can be used in combination therapy with at least one other agent. In some embodiments, a compound as disclosed and described herein and/or topical composition thereof is administered concurrently with the administration of another agent, which may be part of the same topical composition as the compound of the present invention or a different composition. In other embodiments, a topical composition of the present invention is administered prior or subsequent to administration of another agent.

### Methods of Preparation

The compounds of Formula (I), Formula (IIa), Formula (IIb) and Formula (III) disclosed herein may be synthesized by methods known in the art, or by modification of these methods. Ways of modifying the methodology include, among others, temperature, solvent, reagents etc., known to those skilled in the art. In general, during any of the processes for preparation of the compounds disclosed herein, it may be necessary and/or desirable to protect sensitive or reactive groups on any of the molecules concerned. This may be achieved by means of conventional protecting groups, such as those described in Protective Groups in Organic Chemistry (ed. J. F. W. McOmie, Plenum Press, 1973); and P. G. M. Green, T. W. Wutts, Protecting Groups in Organic Synthesis (3rd ed.) Wiley, New York (1999), which are both hereby incorporated herein by reference in their entirety. The protecting groups may be removed at a convenient subsequent stage using methods known from the art. Synthetic chemistry transformations useful in synthesizing applicable compounds are known in the art and include e.g. those described in R. Larock, Comprehensive Organic Transformations, VCH Publishers, 1989, or L. Paquette, ed., Encyclopedia of Reagents for Organic Synthesis, John Wiley and Sons, 1995, which are both hereby incorporated herein by reference in their entirety. Those skilled in the art will be able to recognize modifications of the disclosed syntheses and to devise alternate routes based on the disclosures herein; all such modifications and alternate routes are within the scope of the claims.

The starting materials used in preparing the compounds described herein are often known compounds, or can be synthesized by known methods described in the literature, or are commercially available from various sources well known to those of ordinary skill in the art.

It is recognized that the skilled artisan in the art of organic chemistry can readily carry out the synthesis of many starting materials and subsequent manipulations without further direction, that is, it is well within the scope and practice of the skilled artisan to carry out many desired manipulations. These include reduction of carbonyl compounds to their corresponding alcohols, oxidations, acylations, aromatic substitutions, both electrophilic and nucleophilic, etherifications, esterification, saponification, nitrations, hydrogenations, reductive amination and the like.

Exemplary synthetic methods for preparing compounds of Formula (I) are provided in detail in U.S. 2013/0324557 A1, which is incorporated herein by reference in its entirety. Exemplary synthetic methods for preparing compounds of Formula (IIa), Formula (IIb), and Formula (III) are provided in WO 2014/130582, which is incorporated herein by reference in its entirety. The entirety of each of these documents is herein incorporated by reference.

### EXAMPLES

### Example 1

### Studies of Compounds of Formula (I)

### 1.1) Biological Assay of Compounds of Formula (I)

A mammalian cell line derivative which stably expresses hTRPM8 was used in biological assays in association with testing the present compounds with cool-tasting or -feeling properties (Servant et al. US 2007/0259354 A1 and references cited therein). Typical compound concentrations tested were 100 μM, 30 μM, 10 μM, 3.3 μM, 1.1 μM, 0.37 μM, 0.12 μM, 0.04 μM 0.01 μM and more dilutions for highly potent compounds. The present compounds have shown strong activity as agonists of hTRPM8. Assay results for compounds are illustrated in Table 1.1 below. Specifically, the Examples listed in Table 1.1, i.e., Compounds A1 to Compounds U10 are the specific compounds above that fall within Formula (I).

TABLE 1.1

| Compound | EC50 (uM) | EC50 WS-3 Ratio |
|---|---|---|
| A1 | 0.000009 | 782471 |
| B1 | 0.000001 | 1000000 |
| C1 | 0.000017 | 502141 |
| D1 | 0.000254 | 119614 |
| E1 | 0.000391 | 22099 |
| F1 | 0.000205 | 21178 |
| G1 | 0.00019 | 18711 |
| H1 | 0.000279 | 15169 |
| I1 | 0.000425 | 9981 |
| J1 | 0.000575 | 9607 |
| K1 | 0.000643 | 6336 |
| L1 | 0.00047 | 7808 |
| M1 | 0.0012 | 4571 |
| N1 | 0.0010 | 4432 |
| O1 | 0.0014 | 3821 |
| P1 | 0.0018 | 3444 |
| Q1 | 0.0023 | 1968 |
| R1 | 0.0023 | 1764 |
| S1 | 0.0033 | 886 |
| T1 | 0.0057 | 823 |
| U1 | 0.0077 | 577 |
| V1 | 0.0069 | 630 |
| W1 | 0.0150 | 365 |
| X1 | 0.0232 | 222 |
| Y1 | 0.0197 | 207 |
| Z1 | 0.0211 | 204 |
| A2 | 0.0171 | 165 |
| B2 | 0.0486 | 149 |
| C2 | 0.0319 | 134 |
| D2 | 0.0383 | 134 |
| E2 | 0.0304 | 111 |
| F2 | 0.0525 | 101 |
| G2 | 0.0431 | 96 |
| H2 | 0.0450 | 91 |
| I2 | 0.0522 | 77 |
| G2 | 0.0791 | 76 |
| K2 | 0.0717 | 66 |
| L2 | 0.0619 | 60 |
| M2 | 0.0613 | 58 |
| N2 | 0.625 | 7 |
| O2 | 0.536 | 7 |
| P2 | 0.061 | 47 |
| Q2 | 0.088 | 49 |
| R2 | 0.111 | 37 |
| S2 | 0.112 | 36 |
| T2 | 0.116 | 36 |
| U2 | 0.170 | 32 |
| V2 | 0.128 | 32 |
| W2 | 0.136 | 32 |
| X2 | 0.116 | 30 |
| Y2 | 0.109 | 28 |
| Z2 | 0.363 | 23 |
| A3 | 0.209 | 24 |
| B3 | 0.214 | 22 |
| C3 | 0.178 | 18 |
| D3 | 0.212 | 18 |
| E3 | 0.261 | 18 |
| F3 | 0.534 | 16 |
| G3 | 0.407 | 14 |
| H3 | 0.255 | 14 |
| I3 | 0.422 | 14 |
| J3 | 0.324 | 13 |
| K3 | 0.505 | 13 |
| L3 | 0.378 | 12 |
| M3 | 0.280 | 12 |
| N3 | 0.422 | 12 |
| O3 | 0.558 | 12 |
| P3 | 0.290 | 12 |
| Q3 | 0.351 | 11 |
| R3 | 0.750 | 11 |
| S3 | 0.386 | 10 |
| T3 | 0.422 | 10 |
| U3 | 0.432 | 10 |
| V3 | 0.489 | 9 |
| W3 | 0.371 | 9 |
| X3 | 0.461 | 9 |
| Y3 | 0.962 | 9 |
| Z3 | 0.515 | 8 |
| A4 | 0.599 | 8 |
| B4 | 0.408 | 7 |
| C4 | 1.235 | 3 |
| D4 | 1.786 | 3 |
| E4 | 0.537 | 7 |
| F4 | 0.858 | 7 |
| G4 | 0.614 | 7 |
| H4 | 0.734 | 6 |
| I4 | 0.813 | 6 |
| J4 | 0.622 | 6 |
| K4 | 0.877 | 6 |
| L4 | 0.828 | 6 |
| M4 | 0.892 | 5 |
| N4 | 0.908 | 5 |
| O4 | 0.712 | 5 |
| P4 | 0.693 | 5 |
| Q4 | 0.767 | 5 |
| R4 | 0.904 | 5 |
| S4 | 1.182 | 5 |
| T4 | 0.859 | 5 |
| U4 | 1.049 | 5 |
| V4 | 1.845 | 5 |
| W4 | 1.102 | 4 |
| X4 | 1.360 | 4 |
| Y4 | 0.625 | 4 |
| Z4 | 0.824 | 4 |
| A5 | 1.451 | 4 |
| B5 | 0.637 | 4 |
| C5 | 0.889 | 4 |
| D5 | 0.891 | 4 |
| E5 | 0.726 | 4 |
| F5 | 1.949 | 4 |
| G5 | 1.720 | 3 |
| H5 | 1.230 | 4 |
| I5 | 1.779 | 3 |
| G5 | 1.040 | 3 |
| K5 | 2.161 | 3 |
| L5 | 1.197 | 3 |
| M5 | 1.422 | 3 |
| N5 | 1.175 | 3 |
| O5 | 1.127 | 3 |
| P5 | 2.079 | 3 |
| Q5 | 3.810413 | 1 |
| R5 | 1.401 | 3 |
| S5 | 1.771 | 2 |
| T5 | 1.607 | 2 |
| U5 | 2.386 | 2 |
| V5 | 1.876 | 2 |
| W5 | 2.318 | 2 |
| X5 | 2.173 | 2 |
| Y5 | 2.375 | 2 |
| Z5 | 1.180 | 2 |
| A6 | 2.268 | 2 |
| B6 | 2.173 | 2 |
| C6 | 3.731 | 2 |
| D6 | 2.240 | 2 |
| E6 | 2.002 | 2 |
| F6 | 2.803 | 2 |
| G6 | 2.037 | 2 |
| H6 | 3.740 | 2 |
| I6 | 2.570 | 2 |
| J6 | 2.623 | 2 |
| K6 | 2.262 | 2 |
| L6 | 3.062 | 2 |
| M6 | 2.249 | 2 |
| N6 | 2.619 | 2 |
| O6 | 3.301 | 2 |
| P6 | 2.882 | 2 |
| Q6 | 1.671 | 2 |
| R6 | 2.593 | 1 |
| S6 | 3.444 | 1 |
| T6 | 2.405 | 1 |
| U6 | 2.997 | 1 |
| V6 | 3.289 | 1 |
| W6 | 3.751 | 1 |
| X6 | 3.216 | 1 |
| Y6 | 3.824 | 1 |
| Z6 | 4.629 | 1 |
| A7 | 4.660 | 1 |
| B7 | 3.703 | 1 |
| C7 | 3.470 | 1 |
| D7 | 0.124 | 53 |
| E7 | 5.516541 | 1 |
| F7 | 4.939072 | 1 |
| G7 | 4.285153 | 1 |
| H7 | 5.343864 | 1 |
| I7 | 5.11506 | 1 |
| J7 | 6.197407 | 1 |
| K7 | 4.238856 | 1 |
| L7 | 5.375403 | 1 |
| M7 | 10.98696 | 0.316 |
| N7 | 0.000011 | 1454336 |
| O7 | 0.000002 | 3531839 |
| P7 | 0.000004 | 1200594 |
| Q7 | 0.000006 | 817519 |
| R7 | 0.000026 | 182281 |
| S7 | 0.000266 | 32698 |
| T7 | 0.000397 | 24280 |
| U7 | 0.003001 | 9481 |
| V7 | 0.0007 | 6834 |
| W7 | 0.0018 | 4318 |
| X7 | 0.0011 | 2775 |
| Y7 | 0.0018 | 2799 |
| Z7 | 0.0016 | 2013 |
| A8 | 0.0014 | 1986 |
| B8 | 0.0039 | 813 |
| C8 | 0.0072 | 533 |
| D8 | 0.0082 | 425 |
| E8 | 0.0153 | 399 |
| F8 | 0.0166 | 336 |
| G8 | 0.0153 | 326 |
| H8 | 0.0232 | 183 |
| I8 | 0.0227 | 193 |
| J8 | 0.0313 | 146 |
| K8 | 0.0311 | 144 |
| L8 | 0.0369 | 109 |
| M8 | 0.0440 | 107 |
| N8 | 0.0466 | 87 |
| O8 | 0.0520 | 81 |
| P8 | 0.0405 | 77 |
| Q8 | 0.048 | 69 |
| R8 | 0.058 | 48 |
| S8 | 0.098 | 48 |
| T8 | 0.089 | 48 |
| U8 | 0.093 | 40 |
| V8 | 0.108 | 38 |
| W8 | 0.114 | 37 |
| X8 | 0.130 | 32 |
| Z8 | 0.140 | 32 |
| A9 | 0.157 | 27 |
| B9 | 0.172 | 30 |
| C9 | 0.122 | 27 |
| D9 | 0.151 | 25 |
| E9 | 0.191 | 22 |
| F9 | 0.232 | 22 |
| G9 | 0.259 | 17 |
| H9 | 0.286 | 16 |
| I9 | 0.293 | 13 |
| J9 | 0.352 | 12 |
| K9 | 0.419 | 11 |
| L9 | 0.455 | 12 |
| M9 | 0.361 | 11 |
| N9 | 0.631 | 10 |
| O9 | 0.446 | 9 |
| P9 | 0.560 | 9 |
| Q9 | 0.729 | 8 |
| R9 | 0.750 | 6 |
| S9 | 0.919 | 5 |
| T9 | 0.958 | 5 |
| U9 | 1.040 | 5 |
| V9 | 0.821 | 5 |
| W9 | 0.986 | 5 |
| X9 | 0.931 | 5 |
| Y9 | 1.949 | 4 |
| Z9 | 1.165 | 4 |
| A10 | 1.231 | 4 |
| B10 | 1.227 | 4 |
| C10 | 1.431 | 3 |
| D10 | 0.0535 | 75 |
| E10 | 1.694853 | 3 |
| F10 | 2.161292 | 3 |
| G10 | 2.356718 | 3 |
| H10 | 1.632536 | 3 |
| I10 | 2.916701 | 3 |
| J10 | 1.846245 | 2 |
| K10 | 2.904192 | 2 |
| L10 | 1.924198 | 2 |
| M10 | 1.379 | 3 |
| N10 | 1.985332 | 2 |
| O10 | 3.740034 | 2 |
| P10 | 4.588583 | 1 |
| Q10 | 3.23947 | 1 |
| R10 | 3.035896 | 1 |
| S10 | 6.42262 | 1 |
| T10 | 6.906322 | 1 |
| U10 | 4.85575 | 1 |

### Sensory Studies

Two typical sensory studies are described below followed by a table summarizing sensory results of selected compounds described herein (Table 1.6).

Cool Line Scale Test with Compound Z1:

### Formulation:

All samples made with Low Sodium Buffer (LSB) pH ˜7.1 and contain 0.1% ethanol.

### General Protocol:

Compounds are rated on a 15 point line scale where 45 μM WS-3 (N-Ethyl-p-menthane-3-carboxamide) is ranked as a 5 in cool intensity. In most cases our compounds are tested to determine at what concentration the cooling intensity is equivalent to 45 μM WS-3. In each test, the panelist is presented with a 0 μM control sample, a 45 μM WS-3 control sample and the experimental compound sample and asked to rate the cooling intensity of each sample. Panelists are also asked to rate bitterness. In the table below there was no significant bitterness detected unless otherwise noted. Also, in the table below, n represents the number of tests completed for a given experiment (# panelists×# repetitions).

### Conclusions:

Panelists found 15 μM Compound Z1 was significantly more cooling than 0 μM WS-3 (p<0.05) and not significantly different in cooling than 45 μM WS-3 (p>0.05). There were no significant bitter offtastes in any of the samples (p>0.05). Analytical quantification for 15 μM Compound Z1 from a sample cup from the test was 70% of the expected value, while analytical quantification from the bottle of solution was within the expected range.

TABLE 1.2
Average Cooling, n = 30 (15 Panelists × 2 rep). Tukey's
Value = 1.103 (α = 0.05).

| | Average | Standard | Standard | Tukey |
|---|---|---|---|---|
| Treatment | Score | Deviation (SD) | Error (St Er) | (5%) |
| 0 μM WS-3 | 2.1 | 2.2 | 0.4 | a |
| 15 μM Compound Z1 | 3.4 | 2.1 | 0.4 | b |
| 45 μM WS-3 | 3.8 | 1.9 | 0.3 | b |

TABLE 1.3
Average Bitterness, n = 30 (15 Panelists × 2 rep). Tukey's
Value = 0.442 (α = 0.05).

| Treatment | Average | SD | St Er | Tukey (5%) |
|---|---|---|---|---|
| 0 μM WS-3 | 0.1 | 0.3 | 0.0 | a |
| 15 μM Compound Z1 | 0.3 | 0.6 | 0.1 | a |
| 45 μM WS-3 | 0.3 | 1.0 | 0.2 | a |

Cool Line Scale Test with Compound F1 (3 μM in LSB):

### Formulation:

All samples were prepared with Low Sodium Buffer (LSB) pH ˜7.1 and contain 0.1% ethanol

### Conclusions:

Panelists found 3 μM Compound F1 was significantly more cooling than 004 WS-3 (p<0.05) and not significantly different in cooling than 4504 WS-3 (p>0.05). There were no significant bitter offtastes in any of the samples (p>0.05).

TABLE 1.4
Average Cooling, n = 28 (14 Panelists × 2 rep). Tukey's
Value = 1.359 (α = 0.05).

| Treatment | Average | SD | St Er | Tukey (5%) |
|---|---|---|---|---|
| 0 μM WS-3 | 1.9 | 2.8 | 0.5 | a |
| 3 μM Compound F1 | 5.1 | 1.4 | 0.3 | b |
| 45 μM WS-3 | 5.2 | 2.1 | 0.4 | b |

TABLE 1.5
Average Bitterness, n = 28 (14 Panelists × 2 rep). Tukey's
Value = 0.517 (α = 0.05).

| Treatment | Average | SD | St Er | Tukey (5%) |
|---|---|---|---|---|
| 45 μM WS-3 | 0.3 | 0.8 | 0.1 | a |
| 3 μM Compound F1 | 0.4 | 0.6 | 0.1 | a |
| 0 μM WS-3 | 0.5 | 1.1 | 0.2 | a |

TABLE 1.6
Selected sensory results for compounds of the invention.

| Compound | Sensory Results | n |
|---|---|---|
| E1 | Panelists found 2 μM Compound E1 was significantly | 26 |
| | more cooling than 0 μM WS- 3 and significantly less | |
| | cooling than 45 μM. | |
| F1 | Panelists found 3 μM Compound F1 was significantly | 28 |
| | more cooling than 0 μM WS- 3 (p < 0.05) and not | |
| | significantly different in cooling than 45 μM WS-3 | |
| | (p > 0.05). | |
| J1 | Panelists found 3 μM Compound J1 was significantly | 28 |
| | more cooling than 0 μM WS- 3 (p < 0.05) and not | |
| | significantly different in cooling than 45 μM WS-3 | |
| | (p > 0.05). | |
| N1 | Panelists found 5 μM Compound N1 was not | 22 |
| | significantly different in cooling than 0 μM WS-3 | |
| | and 45 μM WS-3 (p > 0.05), but had an average | |
| | score between 0 μM WS-3 and 45 μM WS-3. | |
| | Panelists found 5 μM Compound N1 had a significant | |
| | bitter offtaste (p < 0.05). | |
| V1 | Panelists found 5 μM Compound V1 was significantly | 28 |
| | more cooling than 0 μM WS- 3 (p < 0.05) and not | |
| | significantly different in cooling than 45 μM WS-3 | |
| | (p > 0.05). | |
| U1 | Panelists found 5 μM Compound U1 was significantly | 24 |
| | more cooling than 0 μM WS- 3 (p < 0.05) and not | |
| | significantly different in cooling than 45 μM WS-3 | |
| | (p > 0.05). | |
| W1 | Panelists found 5 μM Compound W1 was significantly | 22 |
| | more cooling than 0 μM WS- 3 (p < 0.05) and not | |
| | significantly different in cooling than 45 μM WS-3 | |
| | (p > 0.05). | |
| Z1 | Panelists found 15 μM Compound Z1 was significantly | 30 |
| | more cooling than 0 μM WS-3 (p < 0.05) and not | |
| | significantly different in cooling than 45 μM WS-3 | |
| | (p > 0.05). There were no significant bitter offtastes | |
| | in any of the samples (p > 0.05). | |
| B2 | Panelists found 5 μM Compound B2 was significantly | 26 |
| | more cooling than 0 μM WS-3 and significantly less | |
| | cooling than 45 μM WS-3 (p < 0.05). There were no | |
| | significant bitter offtastes in any of the samples (p > | |
| | 0.05). | |
| L2 | Panelists found 15 μM Compound L2 was significantly | 28 |
| | more cooling than 0 μM WS-3 and significantly less | |
| | cooling than 45 μM WS-3 (p < 0.05). | |
| M2 | Panelists found 5 μM Compound M2 was significantly | 30 |
| | more cooling than 0 μM WS-3 (p < 0.05) and not | |
| | significantly different in cooling than 45 μM WS-3 | |
| | (p > 0.05). | |
| T2 | Panelists found 16.7 μM Compound T2 was | 28 |
| | significantly more cooling than 0 μM WS-3 (p < 0.05) | |
| | and not significantly different in cooling than | |
| | 45 μM WS-3 (p > 0.05). | |
| W2 | Panelists found 5 μM Compound W2 was significantly | 30 |
| | more cooling than 0 μM WS- 3 (p < 0.05) and not | |
| | significantly different in cooling than 45 μM WS-3 | |
| | (p > 0.05). | |
| A3 | Panelists found 15 μM Compound A3 was | 30 |
| | significantly more cooling than 0 μM WS-3 (p < 0.05) | |
| | and not significantly different in cooling than 45 μM | |
| | WS-3 (p > 0.05). | |
| I3 | Panelists found 5 μM Compound I3 was not | 30 |
| | significantly different in cooling than 0 μM WS-3 | |
| | (p > 0.05) and significantly less cooling than 45 μM | |
| | WS-3 (p < 0.05). | |
| K3 | Panelists found 15 μM Compound K3 was | 30 |
| | significantly more cooling than 0 μM WS-3 (p < 0.05) | |
| | and not significantly different in cooling than 45 μM | |
| | WS-3 (p > 0.05). | |
| V3 | Panelists found 15 μM Compound V3 was | 30 |
| | significantly more cooling than 0 μM WS-3 (p < 0.05) | |
| | and not significantly different in cooling than 45 μM | |
| | WS-3 (p > 0.05). | |
| P6 | Panelists found 90 μM Compound P6 was not | 16 |
| | significantly different in cooling than 45 μM WS-3 | |
| | (p > 0.05) and significantly more cooling than 0 μM | |
| | WS-3 (p < 0.05). | |
| P7 | Panelists found 3 μM Compound P7 was significantly | 30 |
| | more cooling than 0 μM WS-3 (p < 0.05) and not | |
| | significantly different in cooling than 45 μM WS-3 | |
| | (p > 0.05). | |
| Y7 | Panelists found 3 μM Compound Y7 was significantly | 26 |
| | more cooling than 0 μM WS-3 (p < 0.05) and not | |
| | significantly different in cooling than 45 μM WS-3 | |
| | (p > 0.05). | |
| Q7 | Panelists found 3 μM Q7 was significantly more | 26 |
| | cooling than 0 μM WS-3 (p < 0.05) and not | |
| | significantly different in cooling than 45 μM WS-3 | |
| | (p > 0.05). | |

Biological test results of various compounds have also indicated that the present compounds wherein the hAr is a five-membered heteroaryl are surprisingly much more potent than those compounds wherein the hAr is an aryl or heteroaryl which is not five-membered. Exemplifying data are provided in Tables 1.7 and 1.8 below.

TABLE 1.7.
(T7)

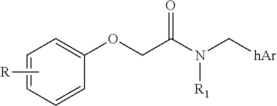

| R | R1 | Ar | EC₅₀ (μM) | EC₅₀ Ratio (WS-3) |
|---|---|---|---|---|
| H | Ethyl | Phenyl | 9.2100 | 0.5 |
| H | Ethyl | Thienyl | 1.4500 | 4.1 |
| 4-Me | Ethyl | Phenyl | 0.7755 | 4.2 |
| 4-Me | Ethyl | Thienyl | 0.0069 | 490.0 |
| 4-Me | 2-Pyridyl | Phenyl | 4.7239 | 0.6 |
| 4-Me | 2-Pyridyl | Thienyl | 0.5053 | 25.4 |
| 4-Me | Methyl | Phenyl | >100 | NA* |
| 4-Me | Methyl | Thienyl | 0.3694 | 14.5 |
| 4-Cl | 2-Pyridyl | Phenyl | NA | NA** |
| 4-Cl | 2-Pyridyl | Thienyl | 2.3182 | 2.4 |
| 4-Br | 2-Pyridyl | Phenyl | 15.5660 | 0.2 |
| 4-Br | 2-Pyridyl | Thienyl | 2.2679 | 2.3 |

*10% at 100 μM.
**22% at 25 μM.

TABLE 1.8.
(T8)

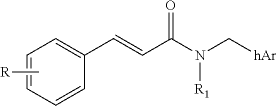

| R | R1 | Ar | EC₅₀ (μM) | EC₅₀ Ration (WS-3) |
|---|---|---|---|---|
| 3,4-methylenedioxy | Ethyl | Phenyl | 1.5000* | nd |
| 3,4-methylenedioxy | Ethyl | Thienyl | >0.001 | 6833.8 |

*According to WO2011/061330 A2 E Table C 3-14.

### Example 2

### Studies of Compounds of Formula (IIa) and (IIb)

### 2.1 Biological Assays of Compounds of Formula (IIa)

A mammalian cell line derivative which stably expresses TRPM8 was used in biological assays in association with testing compounds of Formula (IIa) with cool-tasting or -feeling properties (Servant et al. US 2007/0259354 A1 and references cited therein, which is incorporated herein by reference in its entirety). Typical compound concentrations tested were 100 μM, 30 μM, 10 μM, 3.3 μM, 1.1 μM, 0.37 μM, 0.12 μM, 0.04 μM, 0.01 μM and more dilutions for very potent compounds. The compounds have shown strong activity as agonists of hTRPM8. Assay results for compounds are illustrated in Table 2.1 below. Specifically, the Compounds listed in Table 2.1, e.g., Compounds 1.A1 to Compounds 1.A9 are the specific compounds as described herein that fall within Formula (IIa).

TABLE 2.1

| | hTRPM8 | hTRPM8 | Solubility | |
|---|---|---|---|---|
| | EC50 | EC50 | (μM) | |
| | Ratio | (uM) | LSB | Sensory Results |
| 1.A5 | 25 | 0.123 | | |
| 1.B1 | 10.5 | 0.5642 | | |
| 1.B2 | 2.1 | 2.8113 | | |
| 1.B3 | 36.0 | 0.0850 | 580 | 15 uM < 45 uM WS-3 |
| 1.B4 | 11.4 | 0.4504 | | |
| 1.B5 | 7.0 | 0.6013 | | |
| 1.B6 | 1.3 | 3.4589 | | |
| 1.A9 | 6.1 | 0.6861 | | |
| 1.A8 | 7.0 | 0.5988 | | |
| 1.B7 | 65.2 | 0.0698 | | |
| 1.A7 | 8.5 | 0.4767 | | |
| 1.A6 | 52.5 | 0.0792 | | |
| 1.A4 | 416.0 | 0.0100 | 31 | 5 uM > 45 uM WS-3 |
| 1.B8 | 34.0 | 0.1284 | | |
| 1.B9 | 533.0 | 0.0081 | 117 | 5 uM > 45 uM WS-3 |
| 1.C1 | 9.1 | 0.6552 | | |
| 1.C2 | 3.7 | 1.6314 | | |
| 1.C3 | 15.5 | 0.3092 | | |
| 1.C4 | 3.8 | 1.2538 | | |
| 1.C5 | 9.0 | 0.5892 | | |
| 1.C6 | 13.3 | 0.3987 | | |
| 1.C7 | 3.9 | 1.0666 | | |
| 1.C8 | 24.5 | 0.1859 | | |
| 1.A3 | 46.0 | 0.0870 | 2316 | 10 uM~45 uM WS-3 |
| 1.C9 | 45.0 | 0.0870 | 150 | |
| 1.D1 | 60.5 | 0.0747 | | |
| 1.D2 | 60.7 | 0.0742 | | |
| 1.D3 | 1.7 | 2.3914 | | |
| 1.D4 | 973.4 | 0.0031 | 792 | 2 uM~45 uM WS-3 |
| 1.D5 | 189.0 | 0.0240 | 766 | 5 uM~45 uM WS-3 |
| 1.D6 | 117.3 | 0.0440 | | |
| 1.A2 | 564.0 | 0.0006 | 94 | 10 uM < 45 uM WS-3 |
| 1.D7 | 1.0 | 5.7741 | | |
| 1.D8 | 374.0 | 0.0170 | 639 | 2 uM~45 uM WS-3 |
| 1.A1 | 100.0 | 0.0340 | 337 | 5 uM~45 uM WS-3 |

### 2.2) Sensory Studies of Compounds of Formula (IIb)

Three typical sensory studies are described below each followed by a table summarizing sensory results of selected compounds of the invention that fall within Formula (IIb) (Tables 2.2 to 2.4).

Formulation:

All samples made with Low Sodium Buffer (LSB) pH ˜7.1 and contain 0.1% ethanol.

### General Protocol:

Compounds are rated on a 15 point line scale where 45 μM WS-3 (N-Ethyl-p-menthane-3-carboxamide) is ranked as a 5 in cool intensity. In most cases our compounds are tested to determine at what concentration the cooling intensity is equivalent to 45 μM WS-3. In each test, the panelist is presented with a 0 μM control sample, a 45 μM WS-3 control sample and the experimental compound sample and asked to rate the cooling intensity of each sample. Panelists are also asked to rate bitterness. In the table below there was no significant bitterness detected unless otherwise noted. Also, in the table below, n represents the number of tests completed for a given experiment (# panelists×# repetitions).

### Conclusions:

Panelists found 5 μM of Compound 1.A1 was significantly more cooling than 0 μM WS-3 (p<0.05) and not significantly different in cooling than 45 μM WS-3 (p>0.05). There were no significant bitter offtastes in any of the samples (p>0.05).

TABLE 2.2
Average Cooling, n = 30 (15 Panelists × 2 rep). Tukey's
Value = 0.993 (α = 0.05).

| Treatment | Average | SD | St Er | Tukey (5%) |
|---|---|---|---|---|
| 0 μM WS-3 | 0.8 | 1.8 | 0.3 | a |
| 45 μM WS-3 | 4.4 | 2.4 | 0.4 | b |
| 5 μM Compound 1.A1 | 4.7 | 2.8 | 0.5 | b |

### Conclusions:

Panelists found 10 μM of Compound 1.A3 was significantly more cooling than 0 μM WS-3 (p<0.05) and not significantly different in cooling than 45 μM WS-3 (p>0.05). There were no significant bitter offtastes in any of the samples (p>0.05).

TABLE 2.3
Average Cooling, n = 28 (14 Panelists × 2 rep). Tukey's
Value = 1.078 (α = 0.05).

| Treatment | Average | SD | St Er | Tukey (5%) |
|---|---|---|---|---|
| 0 μM WS-3 | 0.9 | 1.8 | 0.3 | a |
| 10 μM Compound 1.A3 | 4.5 | 2.6 | 0.5 | b |
| 45 μM WS-3 | 5.4 | 1.7 | 0.3 | b |

### Conclusions:

Panelists found 10 μM of compound 1.A2 was significantly more cooling than 0 μM WS-3 (p<0.05) and significantly less cooling than 45 μM WS-3 (p>0.05). There were no significant bitter offtastes in any of the samples (p>0.05).

TABLE 2.4
Average Cooling, n = 28 (14 Panelists × 2 rep). Tukey's
Value = 1.022 (α = 0.05).

| Treatment | Average | SD | St Er | Tukey (5%) |
|---|---|---|---|---|
| 0 μM WS-3 | 0.8 | 1.6 | 0.3 | a |
| 10 μM Compound 1.A2 | 2.7 | 2.1 | 0.4 | b |
| 45 μM WS-3 | 4.1 | 2.0 | 0.4 | c |

### 2.3 Biological Assay of Compounds of Formula (IIb)

A cell line which stably expresses hTRPM8 was used in biological assays in association with testing compounds with cool-tasting or -feeling properties. Typical compound concentrations tested were 100 μM, 30 μM, 10 μM, 3.3 μM, 1.1 μM, 0.37 μM, 0.12 μM, 0.04 μM, 0.01 μM and more dilutions for very potent compounds. The present compounds have shown activity as agonists of hTRPM8. Assay results for compounds are illustrated in the table below. EC₅₀ in micromoles (uM) is presented as well as the activity relative to WS3 (an established, commercially available cooling agent). EC₅₀ ratio is determined by taking the EC₅₀ of the example compound and dividing it by the EC₅₀ of WS3 when screened on the same day in the same screen. A value of 1 indicates the compound is equipotent to WS3 in the assay. A number greater than one indicates how many times more potent the compound is in the assay relative to WS3. It is noted that Compounds 2.A1 to 2. S6 in the table below are compounds described in herein falling with Formula (IIb).

| | EC₅₀ | EC₅₀ Ratio |
|---|---|---|
| Compound | (μM) | (WS3) |
| 2.A1 | 0.135 | 54.6 |
| 2.B1 | 0.374 | 13.5 |
| 2.C1 | 0.510 | 9.9 |
| 2.D1 | 0.459 | 13.2 |
| 2.E1 | 0.898 | 8.0 |
| 2.F1 | 2.881 | 1.6 |
| 2.G1 | 4.366 | 1.2 |
| 2.H1 | 0.192 | 240.6 |
| 2.I1 | 3.312 | 1.2 |
| 2.J1 | 0.593 | 14.2 |
| 2.K1 | 0.074 | 108.0 |
| 2.L1 | 6.379 | 0.7 |
| 2.M1 | 0.321 | 32.5 |
| 2.N1 | 1.091 | 3.5 |
| 2.O1 | 0.227 | 20.5 |
| 2.P1 | 0.333 | 11.5 |
| 2.Q1 | 0.273 | 15.9 |
| 2.R1 | 5.471 | 0.7 |
| 2.S1 | 0.760 | 7.0 |
| 2.T1 | 7.195 | 0.5 |
| 2.U1 | 0.140 | 39.2 |
| 2.V1 | 1.592 | 2.1 |
| 2.W1 | 3.357 | 1.0 |
| 2.X1 | 2.039 | 2.1 |
| 2.Y1 | 1.261 | 3.1 |
| 2.Z1 | 6.678 | 0.7 |
| 2.A2 | 0.253 | 22.1 |
| 2.B2 | 10.115 | 0.5 |
| 2.C2 | 0.493 | 12.5 |
| 2.D2 | 0.087 | 85.9 |
| 2.E2 | 0.293 | 13.0 |
| 2.F2 | 11.712 | 5.4 |
| 2.G2 | 0.621 | 8.8 |
| 2.H2 | 0.447 | 10.4 |
| 2.I2 | 0.927 | 7.7 |
| 2.J2 | 0.694 | 3.9 |
| 2.K2 | 0.724 | 5.5 |
| 2.L2 | 0.547 | 9.1 |
| 2.M2 | 7.542 | 0.5 |
| 2.N2 | 0.294 | 16.5 |
| 2.O2 | 0.588 | 8.0 |
| 2.P2 | 0.006 | 2566.9 |
| 2.Q2 | 0.029 | 202.6 |
| 2.R2 | 0.866 | 5.4 |
| 2.S2 | 1.985 | 2.3 |
| 2.T2 | 2.489 | 2.1 |
| 2.U2 | 0.044 | 178.7 |
| 2.V2 | 0.053 | 83.5 |
| 2.W2 | 0.066 | 77.3 |
| 2.X2 | 0.084 | 69.7 |
| 2.Y2 | 0.089 | 54.5 |
| 2.Z2 | 0.096 | 71.5 |
| 2.A3 | 0.100 | 73.1 |
| 2.B3 | 5.279 | 2810.1 |
| 2.C3 | 0.100 | 49.1 |
| 2.D3 | 0.124 | 33.4 |
| 2.E3 | 0.127 | 53.5 |
| 2.F3 | 0.138 | 44.0 |
| 2.G3 | 0.146 | 32.6 |
| 2.H3 | 0.156 | 34.3 |
| 2.I3 | 0.163 | 33.8 |
| 2.J3 | 0.166 | 32.1 |
| 2.K3 | 0.172 | 30.1 |
| 2.L3 | 0.182 | 25.9 |
| 2.M3 | 0.192 | 240.6 |
| 2.N3 | 0.205 | 24.1 |
| 2.O3 | 0.223 | 21.6 |
| 2.P3 | 0.230 | 38.1 |
| 2.Q3 | 0.233 | 20.3 |
| 2.R3 | 0.236 | 22.8 |
| 2.S3 | 0.246 | 18.1 |
| 2.T3 | 0.268 | 22.6 |
| 2.U3 | 0.284 | 17.2 |
| 2.V3 | 0.286 | 18.6 |
| 2.W3 | 0.292 | 19.6 |
| 2.X3 | 0.305 | 21.9 |
| 2.Y3 | 0.307 | 15.3 |
| 2.Z3 | 0.381 | 12.5 |
| 2.A4 | 0.415 | 8.5 |
| 2.B4 | 0.506 | 12.5 |
| 2.C4 | 0.527 | 9.5 |
| 2.D4 | 0.557 | 7.8 |
| 2.E4 | 0.574 | 9.5 |
| 2.F4 | 0.577 | 8.0 |
| 2.G4 | 0.580 | 7.4 |
| 2.H4 | 0.625 | 8.2 |
| 2.I4 | 0.705 | 6.4 |
| 2.J4 | 0.728 | 6.4 |
| 2.K4 | 0.734 | 5.8 |
| 2.L4 | 0.766 | 6.7 |
| 2.M4 | 0.783 | 6.6 |
| 2.N4 | 0.794 | 5.9 |
| 2.O4 | 0.807 | 6.0 |
| 2.P4 | 0.827 | 7.9 |
| 2.Q4 | 0.859 | 6.2 |
| 2.R4 | 0.945 | 4.6 |
| 2.S4 | 0.968 | 5.0 |
| 2.T4 | 0.971 | 3.5 |
| 2.U4 | 1.116 | 3.0 |
| 2.V4 | 1.154 | 3.1 |
| 2.W4 | 1.165 | 4.6 |
| 2.X4 | 1.204 | 4.4 |
| 2.Y4 | 1.263 | 3.9 |
| 2.Z4 | 1.404 | 3.3 |
| 2.A5 | 1.640 | 2.1 |
| 2.B5 | 1.470 | 2.2 |
| 2.C5 | 1.484 | 2.5 |
| 2.D5 | 1.503 | 3.4 |
| 2.E5 | 1.529 | 2.9 |
| 2.F5 | 1.722 | 2.8 |
| 2.G5 | 1.836 | 2.6 |
| 2.H5 | 1.902 | 2.5 |
| 2.I5 | 2.024 | 2.5 |
| 2.J5 | 2.064 | 2.4 |
| 2.K5 | 2.074 | 2.3 |
| 2.L5 | 2.222 | 2.1 |
| 2.M5 | 2.266 | 1.9 |
| 2.N5 | 2.461 | 1.9 |
| 2.O5 | 2.526 | 1.4 |
| 2.P5 | 2.596 | 1.8 |
| 2.Q5 | 3.274 | 1.3 |
| 2.R5 | 3.297 | 1.1 |
| 2.S5 | 3.365 | 0.9 |
| 2.T5 | 3.457 | 1.0 |
| 2.U5 | 3.719 | 0.9 |
| 2.V5 | 3.863 | 1.3 |
| 2.W5 | 3.873 | 1.2 |
| 2.X5 | 3.958 | 1.1 |
| 2.Y5 | 4.199 | 0.9 |
| 2.Z5 | 4.412 | 1.5 |
| 2.A6 | 4.424 | 1.0 |
| 2.B6 | 4.429 | 0.8 |
| 2.C6 | 4.655 | 1.0 |
| 2.D6 | 5.712 | 0.6 |
| 2.E6 | 6.556 | 0.6 |
| 2.F6 | 6.639 | 0.7 |
| 2.G6 | 8.072 | 0.5 |
| 2.H6 | 8.191 | 0.8 |
| 2.I6 | 8.443 | 0.5 |
| 2.J6 | 0.0004 | 13841.0 |
| 2.K6 | 0.134 | 19.7 |
| 2.L6 | 0.152 | 27.5 |
| 2.M6 | 0.749 | 6.9 |
| 2.N6 | 0.089 | 61.9 |
| 2.O6 | 0.161 | 48.2 |
| 2.P6 | 0.059 | 84.8 |
| 2.Q6 | 0.231 | 24.4 |
| 2.R6 | 0.036 | 67.3 |
| 2.S6 | 12.667 | 0.5 |

### 2.4) Sensory Studies of Compounds of Formula (IIb)

Sensory studies were conducted for representative compounds falling within Formula (IIb) and the results are summarized in the table below. The results are presented relative to a known concentration of WS-3.

| Compound | Sensory Results |
|---|---|
| 2.U2 | 30 uM~45 uM WS-3 |
| 2.Q2 | 30 uM~45 uM WS-3 |
| 2.K3 | 30 uM~45 uM WS-3 |
| 2.Z2 | 10 uM~45 uM WS-3 |
| 2.J3 | 15 uM~45 uM WS-3 |
| 2.T3 | 15 uM < 45 uM WS-3 |
| 2.P3 | 15 uM < 45 uM WS-3 |
| 2.R3 | 15 uM~45 uM WS-3 |
| 2.P2 | 10 uM~45 uM WS-3 |
| 2.J6 | 15 uM~45 uM WS-3 |
| 2.H1 | 10 uM~60 uM WS-3 |
| 2.G3 | 15 uM < 45 uM WS-3 |
| 2.N3 | 15 uM < 45 uM WS-3 |
| 2.V2 | 10 uM~45 uM WS-3 |
| 2.A1 | 10 uM < 45 uM WS-3 |
| 2.C1 | 50 uM < 45 uM WS-3 |
| 2.K1 | 10 uM~45 uM WS-3 |
| 2.C2 | 80 uM > 45 uM WS-3 |

A more specific detailed example of the sensory evaluation of selected examples are also presented below. Data generated in this fashion was used to generate the sensory summary presented in the table above.

Line Scale test with 10 uM Compound 2.H1 in LSB at pH 7.1:
10 μM Compound 2.H1 in LSB at pH 7.1 was not significantly different in average cool intensity from 60 μM 195001 (WS-3) (p<0.05)
10 μM Compound 2.H1 in LSB and 60 μM 195001 (WS-3) in LSB at pH 7.1 were significantly higher in cool intensity than LSB (p<0.05)

TABLE 2.5
Average cool scores, n = 10 (5 × 2 Rep) Tukey's
Value = 1.14 (α = 0.05), 0.98 (α = 0.1).

| | Ave | | St | Tukey | Tukey | Off-Taste |
|---|---|---|---|---|---|---|
| Test sample | Cool | SD | Er | (5%) | (10%) | (x number of panelists) |
| Low Sodium | 1.4 | 1.7 | 0.5 | a | a | |
| Buffer (LSB) | | | | | | |
| 10 μM | 3.6 | 1.6 | 0.5 | b | b | Bitter x2, Linger x2 |
| Compound | | | | | | |
| 2.H1 | | | | | | |
| 60 μM WS-3 | 3.7 | 1.6 | 0.5 | b | b | Linger x2, Tingly |

### Example 3

### Studies of Compounds of Formula (III)

### 3.1 Biological Assay of Compounds of Formula (III)

A mammalian cell line derivative which stably expresses TRPM8 was used in biological assays in association with testing the present compounds with cool-tasting or -feeling properties (Servant et al. US 2007/0259354 A1 and references cited therein, which is incorporated herein by reference in their entirety). Typical compound concentrations tested were 50 μM, 20 μM, 10 μM, 5 μM, 2 μM, 1 μM, 0.5 μM, 0.1 μM, 0.05 μM, 0.01 μM, and other concentration points in between. The present compounds have shown strong activity as agonists of hTRPM8. Assay results for compounds are illustrated in Table 3.1 below. Specifically, the Compounds listed in Table 3.1, i.e., Compounds 3.A1 to Compounds 3.G1 are specific compounds described above falling within Formula (III).

TABLE 3.1

| | EC50 | EC50 Ratio | Observed |
|---|---|---|---|
| Example | (uM) | (WS3) | [m/z + 1] |
| 3.A1 | 0.013 | 581.6 | 304.2 |
| 3.A2 | 0.010 | 577.8 | 288.2 |
| 3.A3 | 0.028 | 221.2 | 320.2 |
| 3.A4 | 0.041 | 132.4 | 294.1 |
| 3.A5 | 0.056 | 116.2 | 306.2 |
| 3.A6 | 0.080 | 99.6 | 306.2 |
| 3.A7 | 0.056 | 86.8 | 288.2 |
| 3.A8 | 0.061 | 84.4 | 300.2 |
| 3.A9 | 0.087 | 66.5 | 320.2 |
| 3.A10 | 0.059 | 62.5 | 294.1 |
| 3.B1 | 0.105 | 45.5 | 304.2 |
| 3.B2 | 0.138 | 38.1 | 288.2 |
| 3.B3 | 0.363 | 34.1 | 302.2 |
| 3.B4 | 0.230 | 26.5 | 289.2 |
| 3.B5 | 0.161 | 25.0 | 286.2 |
| 3.B6 | 0.193 | 20.6 | 277.2 |
| 3.B7 | 0.257 | 18.6 | 306.2 |
| 3.B8 | 0.420 | 16.3 | 302.2 |
| 3.B9 | 0.398 | 15.3 | 306.2 |
| 3.B10 | 0.309 | 13.7 | 314.2 |
| 3.C1 | 0.254 | 13.0 | 289.2 |
| 3.C2 | 0.471 | 12.3 | 294.1 |
| 3.C3 | 0.591 | 11.6 | 302.2 |
| 3.C4 | 0.449 | 10.0 | 277.2 |
| 3.C5 | 0.504 | 9.4 | 304.2 |
| 3.C6 | 0.489 | 9.2 | 294.1 |
| 3.C7 | 0.662 | 8.2 | 302.2 |
| 3.C8 | 1.369 | 5.5 | 302.2 |
| 3.C9 | 0.621 | 5.3 | 290.2 |
| 3.C10 | 0.954 | 4.7 | 274.2 |
| 3.D1 | 1.085 | 4.7 | 296.2 |
| 3.D2 | 1.914 | 4.5 | 276.2 |
| 3.D3 | 1.035 | 4.5 | 274.2 |
| 3.D4 | 1.427 | 4.3 | 318.2 |
| 3.D5 | 2.181 | 4.1 | 288.2 |
| 3.D6 | 2.348 | 3.9 | 282.1 |
| 3.D7 | 1.026 | 3.9 | 300.2 |
| 3.D8 | 1.395 | 3.9 | 274.2 |
| 3.D9 | 2.348 | 3.8 | 276.2 |
| 3.D10 | 2.362 | 3.5 | 300.1 |
| 3.E1 | 1.555 | 3.1 | 282.1 |
| 3.E2 | 1.337 | 2.8 | 308.1 |
| 3.E3 | 1.942 | 2.8 | 272.1 |
| 3.E4 | 1.781 | 2.7 | 300.2 |
| 3.E5 | 1.211 | 2.5 | 280.1 |
| 3.E6 | 4.468 | 2.5 | 296.1 |
| 3.E7 | 2.156 | 1.9 | 320.2 |
| 3.E8 | 4.740 | 1.9 | 296.1 |
| 3.E9 | 3.161 | 1.8 | 334.2 |
| 3.E10 | 2.923 | 1.7 | 308.1 |
| 3.F1 | 2.385 | 1.6 | 274.2 |
| 3.F2 | 1.784 | 1.6 | 280.1 |
| 3.F3 | 2.863 | 1.4 | 280.2 |
| 3.F4 | 5.938 | 1.4 | 276.2 |
| 3.F5 | 3.084 | 1.4 | 289.2 |
| 3.F6 | 3.213 | 1.3 | 320.2 |
| 3.F7 | 3.507 | 1.2 | 308.1 |
| 3.F8 | 4.173 | 1.1 | 290.2 |
| 3.F9 | 3.391 | 1.0 | 286.2 |
| 3.F10 | 6.103 | 1.0 | 318.2 |
| 3.G1 | 6.242 | 1.0 | 275.2 |

### 3.2) Sensory Studies of Compounds of Formula (III)

Two typical sensory studies are described below each followed by a table summarizing sensory results of selected compounds within Formula (III) (Tables 3.2 and 3.3).

### Formulation:

All samples made with Low Sodium Buffer (LSB) pH ˜7.1 and contain 0.1% ethanol.

### General Protocol:

The test is a cool line scale test with timed 30 seconds interval. Compounds are rated on a 15 point line scale where 45 μM WS-3 (N-Ethyl-p-menthane-3-carboxamide) is ranked as a 5 in cool intensity. The present compound was tested to determine at what concentration the cooling intensity is equivalent to 45 μM WS-3. In each test, the panelist was presented with a 0 μM control sample, a 45 μM WS-3 control sample and the experimental compound sample and asked to rate the cooling intensity of each sample. Panelists were also asked to rate bitterness. In the table below there was no significant bitterness detected unless otherwise noted. Also, in the table below, n represents the number of tests completed for a given experiment (# panelists×# repetitions).

### Conclusions:

Panelists found 5 μM COMPOUND 3.2 was significantly more cooling than 0 μM WS-3 (p<0.05) and not significantly different in cooling than 45 μM WS-3 (p>0.05). There were no significant bitter offtastes in any of the samples (p>0.05).

TABLE 3.2
Average Cooling, n = 30 (15 Panelists × 2 rep). Tukey's
Value = 0.926 (α = 0.05).

| Treatment | Average | SD | St Er | Tukey (5%) |
|---|---|---|---|---|
| 0 μM WS-3 | 0.7 | 1.5 | 0.3 | a |
| 5 μM Compound 3.2 | 4.0 | 1.8 | 0.3 | b |
| 45 μM WS-3 | 4.5 | 1.5 | 0.3 | b |

TABLE 3.3
Average Bitterness, n = 30 (15 Panelists × 2 rep). Tukey's
Value = 0.317 (α = 0.05).

| Treatment | Average | SD | St Er | Tukey (5%) |
|---|---|---|---|---|
| 0 μM WS-3 | 0.2 | 0.5 | 0.1 | a |
| 5 μM Compound 3.2 | 0.4 | 0.8 | 0.1 | a |
| 45 μM WS-3 | 0.5 | 1.1 | 0.2 | a |

TABLE 3.4
summarizes addition sensory studies that
were conducted on these compounds.

| Example | Sensory result | n |
|---|---|---|
| 3.A1 | Panelists found 5 uM 58758115 was significantly more | 24 |
| | cooling than 0 uM WS-3 (p < 0.05) and not significantly | |
| | different in cooling 45 uM WS-3 (p > 0.05) | |
| 3.A2 | Panelists found 5 uM 57962022 was significantly more | 30 |
| | cooling than 0 uM WS-3 (p < 0.05) and not significantly | |
| | different in cooling 45 uM WS-3 (p > 0.05) | |
| 3.A6 | Panelists found 5 uM 58751264 was significantly more | 20 |
| | cooling than 0 uM WS-3 (p < 0.05) and not significantly | |
| | different in cooling 45 uM WS-3 (p > 0.05) | |
| 3.B2 | Panelists found 5 uM 58750445 was significantly more | 24 |
| | cooling than 0 uM WS-3 (p < 0.05) and not significantly | |
| | different in cooling 45 uM WS-3 (p > 0.05) | |
| 3.C2 | Panelists found 7 uM 59227831 was significantly more | 32 |
| | cooling than 0 uM WS-3 (p < 0.05) and not significantly | |
| | different in cooling than 45 uM WS-3 (p > 0.05) | |

### Example 4

### Sensory Studies of Topically Applied Compound 101

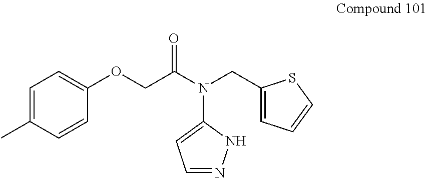 [SMILES: Cc1ccc(OCC(=O)N(Cc2cccs2)c2ccn[nH]2)cc1]

The response of subjects to topically applied Compound 101 was assessed. Subjects were exposed to a solid formulation of Compound 101 in small quantities. Upon exposure, all subjects reported a “tingling” and “cooling” sensation. In another assessment, subjects were exposed to a dilute airborne microparticulate Compound 101. Subjects reported that even in minute quantities, the composition exhibited a “cooling smell” and induced “tingling sensation” on the skin.

1. A topical composition, comprising a carrier and a compound of Formula 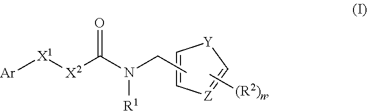
wherein
Ar is optionally substituted aryl, optionally substituted carbocyclyl, or optionally substituted heteroaryl;
Y is oxygen or sulfur;
Z is nitrogen or CR;
R is hydrogen or lower alkyl;
X¹-X² is O—CR^{2a}R^{2b}, CHR³—CHR⁴, or CR⁵═CR⁶;
R^{2a}, R^{2b}, R³, R⁴, R⁵, and R⁶ are independently hydrogen or lower alkyl;
R¹ is an optionally substituted five-membered heteroaryl;
n is 0, 1, 2, or 3; and
each R² is independently optionally substituted alkyl, optionally substituted heteroalkyl, optionally substituted alkenyl, alkoxy, hydroxyl, amino, N-alkyl amino, N-dialkyl amino, halo, nitro, cyano, acyl, carboxyl, carboxyl ester, or amide,
wherein each optional substituent is selected from the group consisting of alkyl, heteroalkyl, alkenyl, alkoxy, hydroxyl, amino, N-alkyl amino, N-dialkyl amino, halo, nitro, cyano, acyl, carboxyl, carboxyl ester, or amide; or two substituents, together with the atoms to which they are attached, form a carbocyclyl optionally substituted with alkyl or alkoxy; or two substituents, together with the atoms to which they are attached, form a heterocyclyl containing one or more heteroatom(s) selected from nitrogen, oxygen, and sulfur.

2. The composition of claim 1, wherein the compound of Formula (I) is selected from the group consisting of: 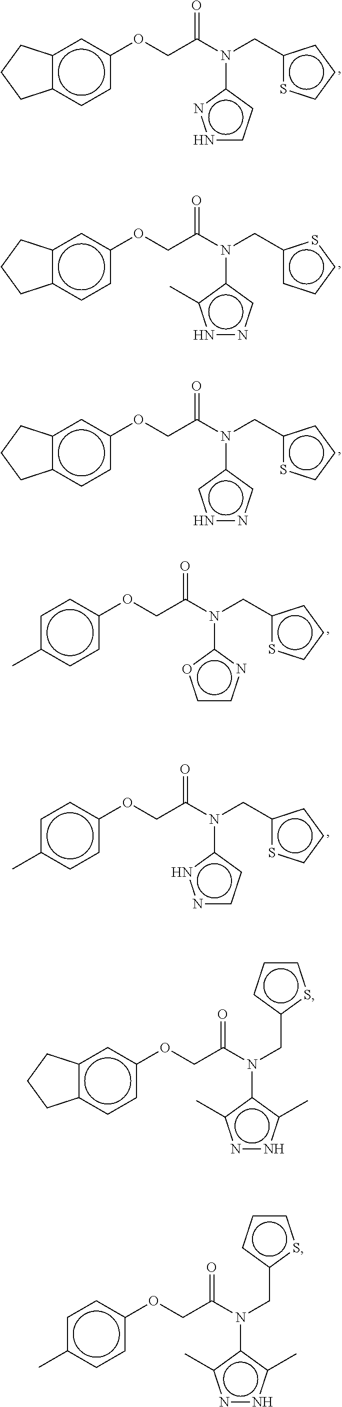 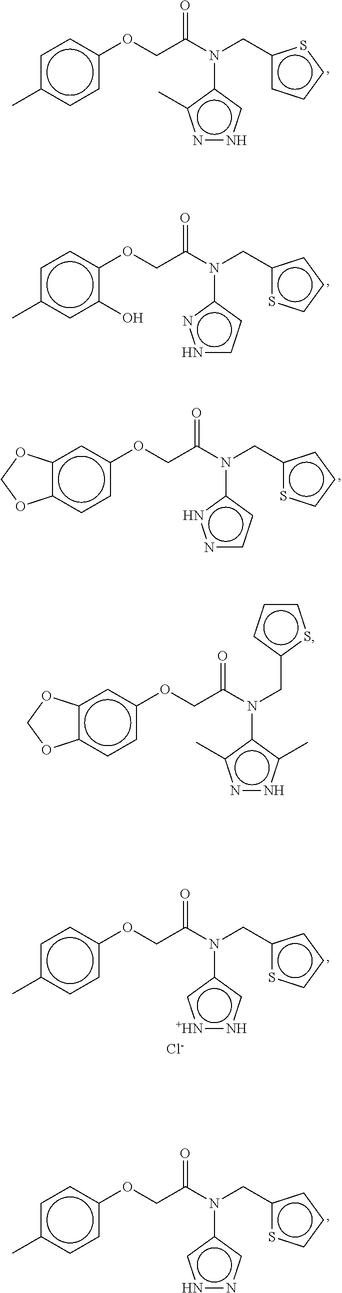 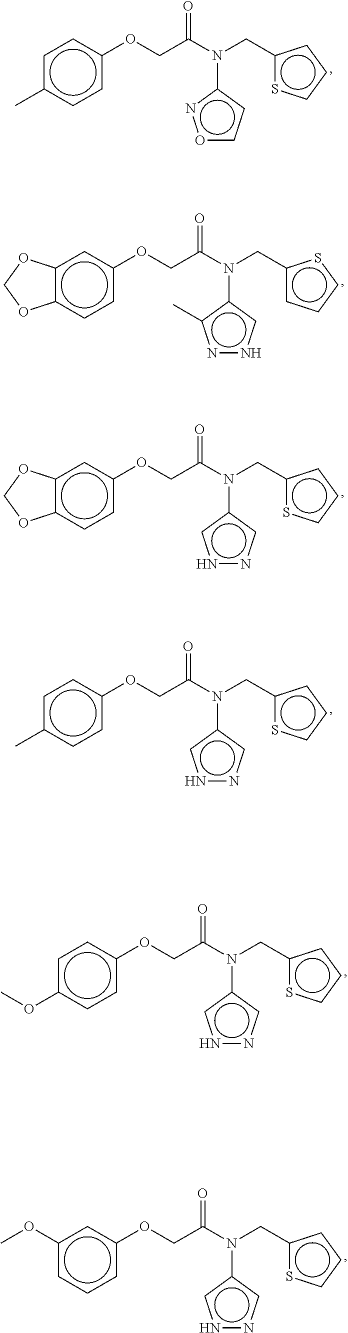 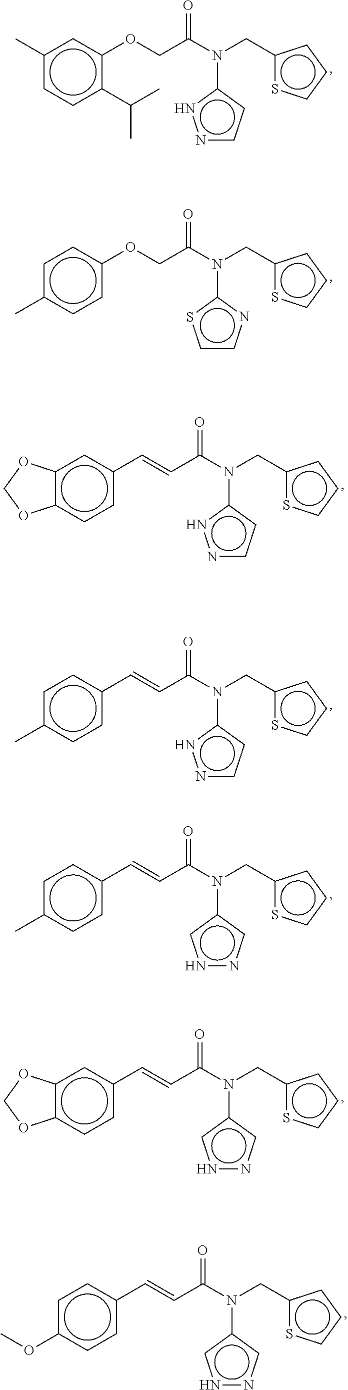 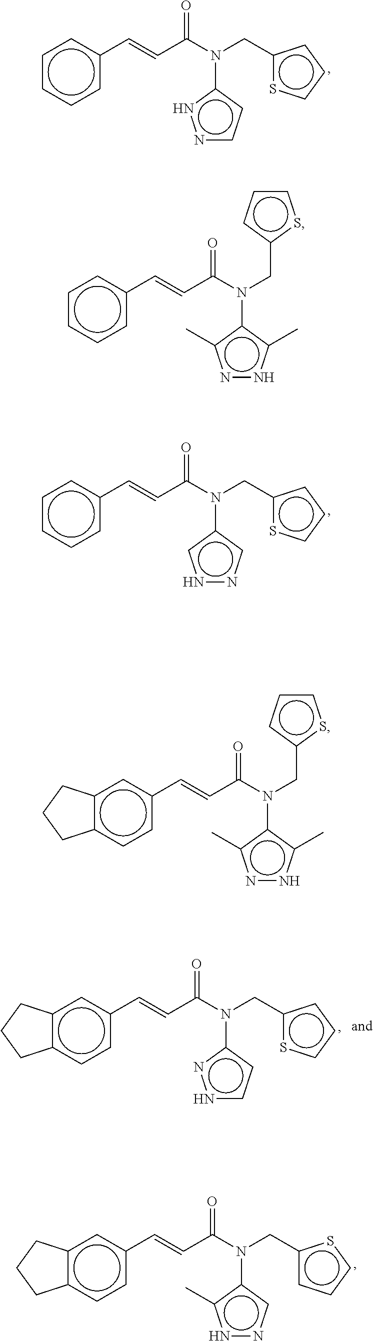
or a salt or solvate thereof.

3. The composition of claim 1, wherein the compound of Formula (I) is 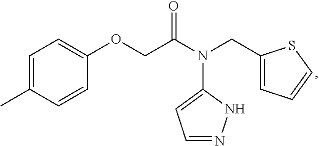 [SMILES: Cc1ccc(OCC(=O)N(Cc2cccs2)c2ccn[nH]2)cc1]
or a salt or solvate thereof.

4. The composition of claim 1, wherein the compound of Formula (I) is selected from the group consisting of 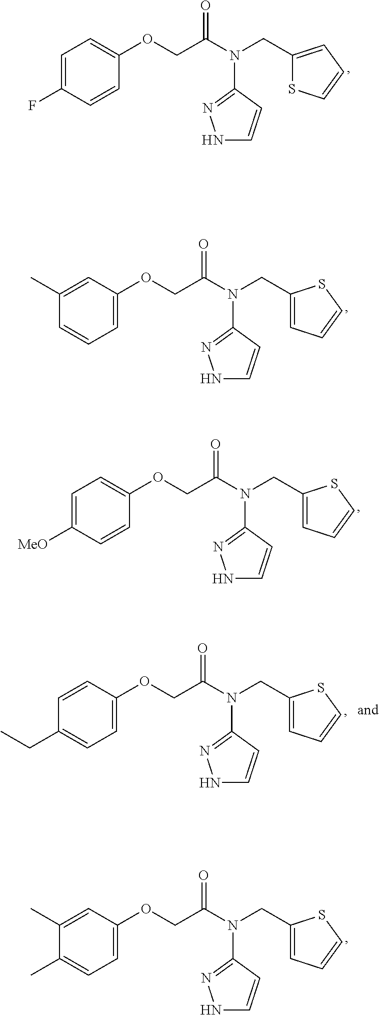 [SMILES: O=C(COc1ccc(F)cc1)N(Cc1cccs1)c1cc[nH]n1 ; Cc1cccc(OCC(=O)N(Cc2cccs2)c2cc[nH]n2)c1 ; COc1ccc(OCC(=O)N(Cc2cccs2)c2cc[nH]n2)cc1 ; CCc1ccc(OCC(=O)N(Cc2cccs2)c2cc[nH]n2)cc1 ; Cc1ccc(OCC(=O)N(Cc2cccs2)c2cc[nH]n2)cc1C]
or a salt or solvate thereof.

5. A topical composition comprising a carrier and a compound of Formula (IIa) or (IIb): 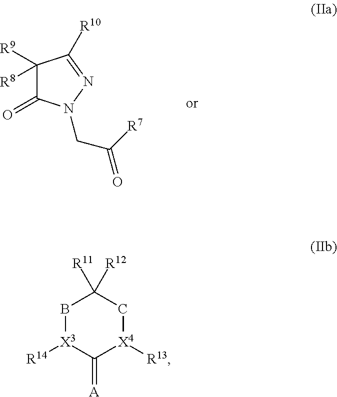
or a salt or solvate thereof;
wherein
R⁷ is optionally substituted aryl, optionally substituted carbocyclyl, optionally substituted heteroaryl, or optionally substituted heterocyclyl;
R⁸ and R⁹ are independently selected from the group consisting of optionally substituted alkyl, optionally substituted alkenyl, optionally substituted aryl, optionally substituted carbocyclyl, optionally substituted heteroaryl, and optionally substituted heterocyclyl;
R¹⁰ is optionally substituted alkyl, optionally substituted alkenyl, optionally substituted aryl, optionally substituted carbocyclyl, optionally substituted heteroaryl, or optionally substituted heterocyclyl;
either R¹¹ or R¹² is optionally substituted C₁-C₃ alkyl; and the remaining R¹¹ or R¹² is selected from the group consisting of optionally substituted alkyl, optionally substituted alkenyl, optionally substituted alkynyl, optionally substituted alkoxy, optionally substituted alkylaryl, optionally substituted alkoxyaryl, optionally substituted aryl, optionally substituted aryloxy, optionally substituted heteroalkyl, optionally substituted carbocyclyl, or optionally substituted heterocylcyl; or alternatively, R¹¹ and R¹², taken together with the atoms to which they are attached, form an optionally substituted carbocyclyl;
X³ and X⁴ are independently CH or N; provided that X³ and X⁴ are not both CH;
R¹³ and R¹⁴ are independently selected from the group consisting of hydrogen, halogen, hydroxyl, cyano, carboxy, optionally substituted C₁-C₈ alkyl, optionally substituted alkenyl, optionally substituted alkynyl, optionally substituted heteroalkyl, optionally substituted alkylaryl, optionally substituted alkoxyaryl, optionally substituted aryl, optionally substituted aryloxy, optionally substituted heteroaryl, optionally substituted heteroaryloxy, optionally substituted carbocyclyl, or optionally substituted heterocyclyl, -alkylene-carbonyl-aryl, -alkylene-carbonyl-heteroaryl, -alkylene-carbonyl-(substituted aryl), -alkylene-carbonyl-(substituted heteroaryl), -alkylene-carbonyl-O-aryl, -alkylene-carbonyl-O-(substituted aryl), -alkylene-carbonyl-NR¹⁵-aryl, -alkylene-carbonyl-NR¹⁵-(substituted aryl), -alkylene-carbonyl-O-heteroaryl, -alkylene-carbonyl-O-(substituted heteroaryl), -alkylene-carbonyl-NR¹⁵-heteroaryl, -alkylene-carbonyl-NR¹⁵-(substituted heteroaryl), 9R¹⁵, and NR¹⁵R¹⁶; or alternatively, X³ and R¹⁴, or X⁴ and R¹³, taken together, is independently O or S;
A is O, S, or NR¹¹;
B and C are independently CH₂, C═O, or a covalent bond; provided that B and C are not both covalent bonds; and
R¹⁵ and R¹⁶ are independently selected from the group consisting of hydrogen, optionally substituted alkyl, optionally substituted aryl, optionally substituted arylalkyl, optionally substituted heteroalkyl, optionally substituted heteroaryl, optionally substituted heteroarylalkyl, optionally substituted acylamido, and optionally substituted diacylamido; or alternatively, R¹⁵ and R¹⁶, together with the atoms to which they are bonded, form an optionally substituted cycloheteroalkyl.

6. The composition of claim 4, wherein the compound of Formula (IIa) or (IIb) is selected from the group consisting of:

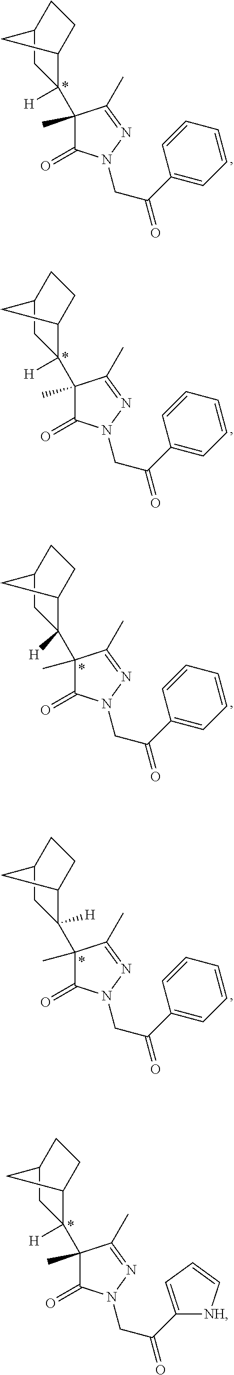 [SMILES: CC1=NN(CC(=O)c2ccccc2)C(=O)[C@]1(C)C1CC2CCC1C2 ; CC1=NN(CC(=O)c2ccccc2)C(=O)[C@@]1(C)C1CC2CCC1C2 ; CC1=NN(CC(=O)c2ccccc2)C(=O)C1(C)[C@@H]1CC2CCC1C2 ; CC1=NN(CC(=O)c2ccccc2)C(=O)C1(C)[C@H]1CC2CCC1C2 ; CC1=NN(CC(=O)c2ccc[nH]2)C(=O)[C@]1(C)C1CC2CCC1C2]

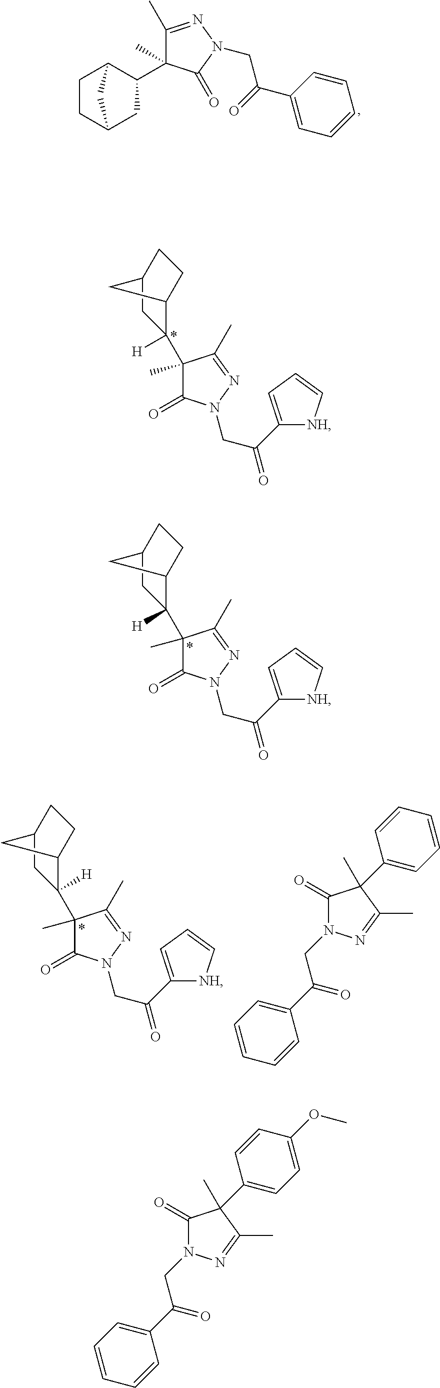 [SMILES: CC1=NN(CC(=O)c2ccccc2)C(=O)[C@@]1(C)[C@@H]1C[C@H]2CC[C@@H]1C2 ; CC1=NN(CC(=O)c2ccc[nH]2)C(=O)[C@@]1(C)C1CC2CCC1C2 ; CC1=NN(CC(=O)c2ccc[nH]2)C(=O)C1(C)[C@@H]1CC2CCC1C2 ; CC1=NN(CC(=O)c2ccc[nH]2)C(=O)C1(C)[C@H]1CC2CCC1C2 ; CC1=NN(CC(=O)c2ccccc2)C(=O)C1(C)c1ccccc1 ; COc1ccc(C2(C)C(=O)N(CC(=O)c3ccccc3)N=C2C)cc1]

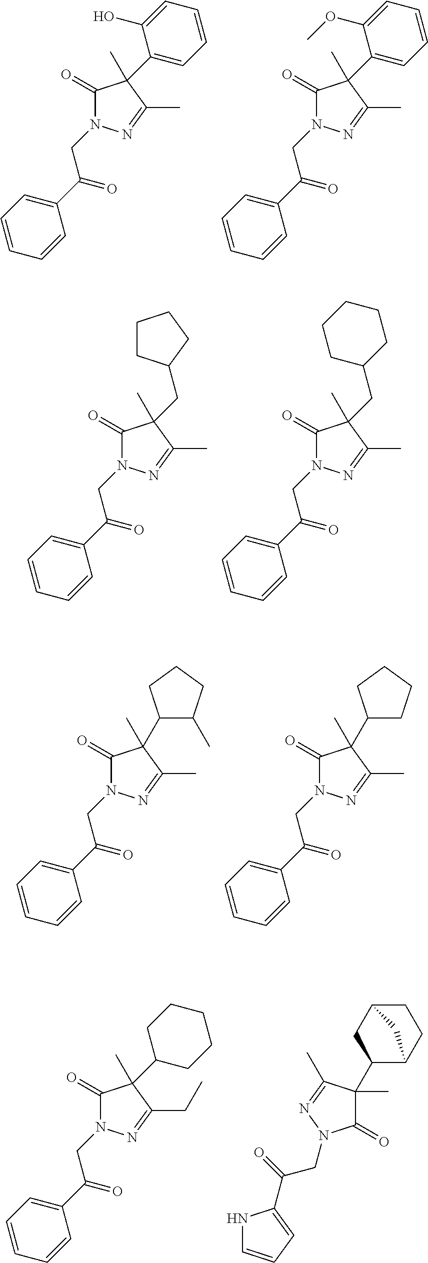

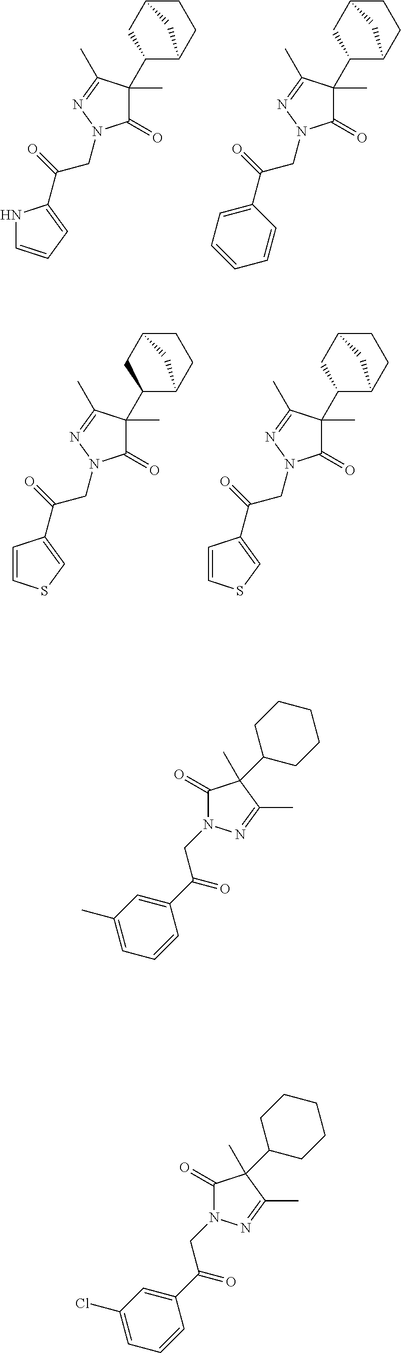 [SMILES: CC1=NN(CC(=O)c2ccc[nH]2)C(=O)C1(C)[C@@H]1C[C@H]2CC[C@@H]1C2 ; CC1=NN(CC(=O)c2ccccc2)C(=O)C1(C)[C@@H]1C[C@H]2CC[C@@H]1C2 ; CC1=NN(CC(=O)c2ccsc2)C(=O)C1(C)[C@H]1C[C@H]2CC[C@@H]1C2 ; CC1=NN(CC(=O)c2ccsc2)C(=O)C1(C)[C@@H]1C[C@H]2CC[C@@H]1C2 ; CC1=NN(CC(=O)c2cccc(C)c2)C(=O)C1(C)C1CCCCC1 ; CC1=NN(CC(=O)c2cccc(Cl)c2)C(=O)C1(C)C1CCCCC1]

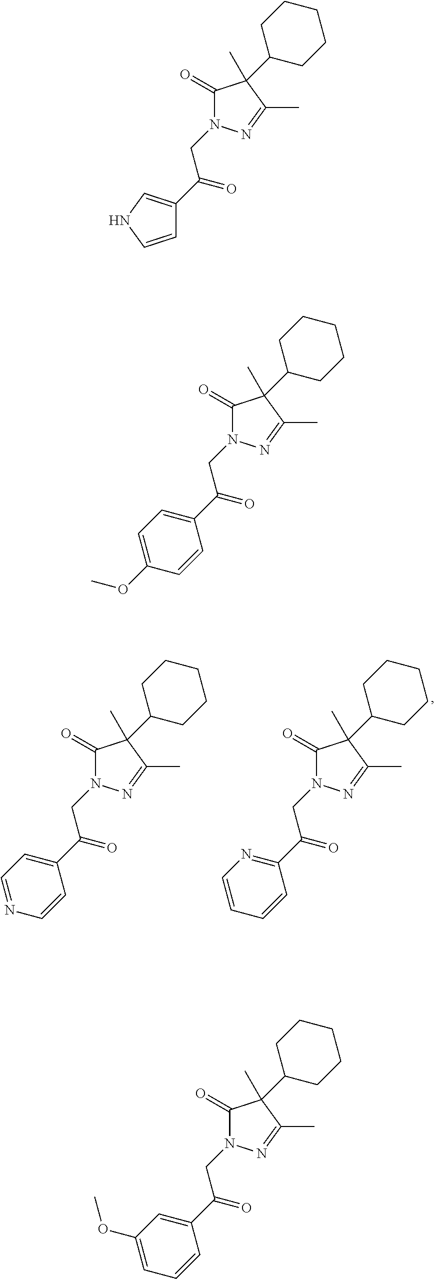 [SMILES: CC1=NN(CC(=O)c2cc[nH]c2)C(=O)C1(C)C1CCCCC1 ; COc1ccc(C(=O)CN2N=C(C)C(C)(C3CCCCC3)C2=O)cc1 ; CC1=NN(CC(=O)c2ccncc2)C(=O)C1(C)C1CCCCC1 ; CC1=NN(CC(=O)c2ccccn2)C(=O)C1(C)C1CCCCC1 ; COc1cccc(C(=O)CN2N=C(C)C(C)(C3CCCCC3)C2=O)c1]

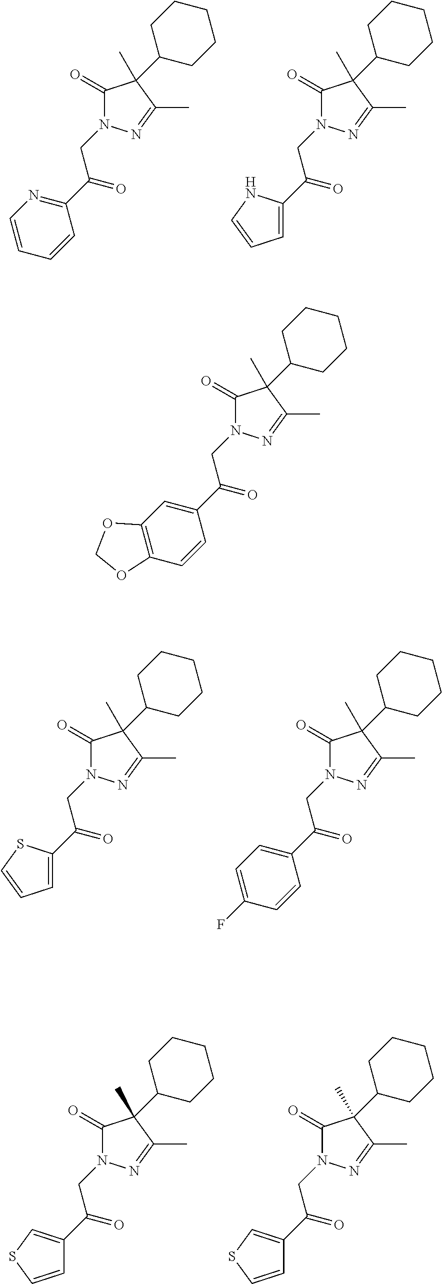

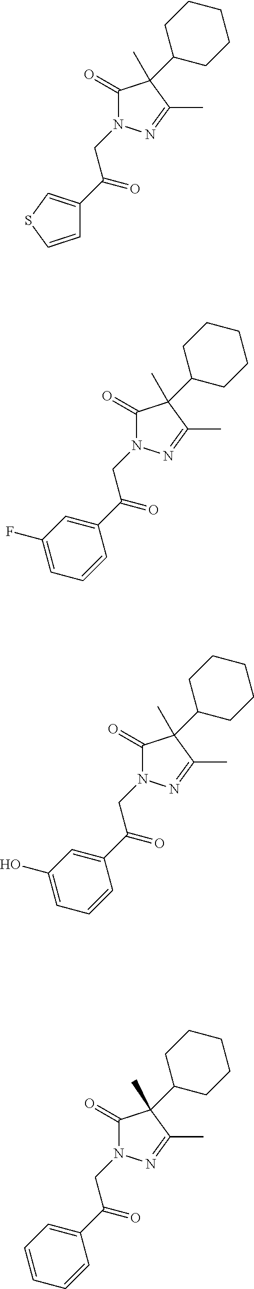 [SMILES: CC1=NN(CC(=O)c2ccsc2)C(=O)C1(C)C1CCCCC1 ; CC1=NN(CC(=O)c2cccc(F)c2)C(=O)C1(C)C1CCCCC1 ; CC1=NN(CC(=O)c2cccc(O)c2)C(=O)C1(C)C1CCCCC1 ; CC1=NN(CC(=O)c2ccccc2)C(=O)[C@]1(C)C1CCCCC1]

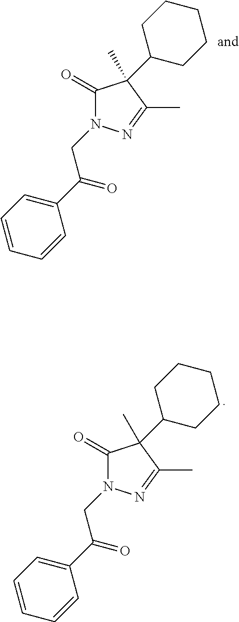 [SMILES: CC1=NN(CC(=O)c2ccccc2)C(=O)[C@@]1(C)C1CCCCC1 ; CC1=NN(CC(=O)c2ccccc2)C(=O)C1(C)C1CCCCC1]

7. The composition of claim 4, wherein the compound of Formula (IIa) or (IIb) is selected from the group consisting of:

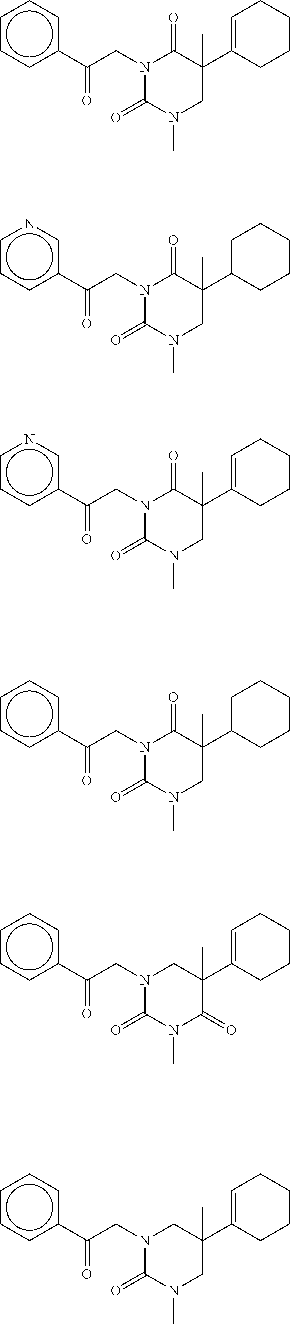

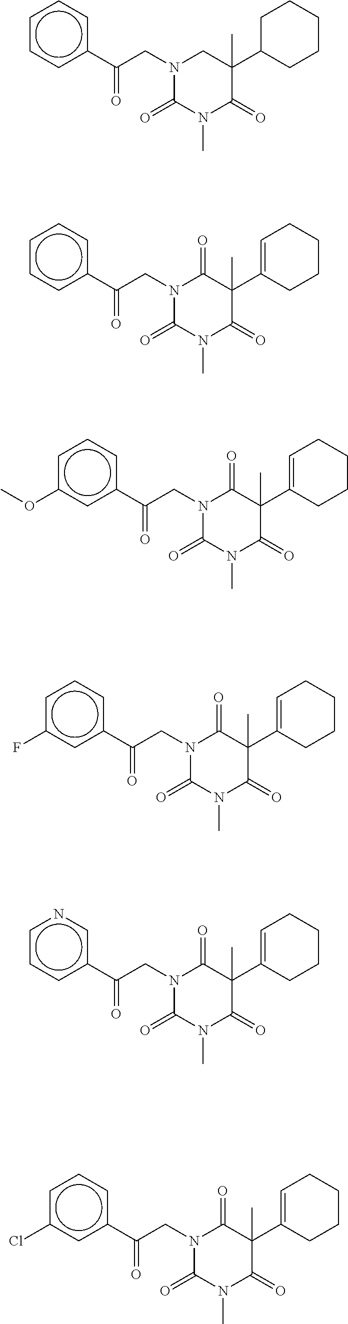

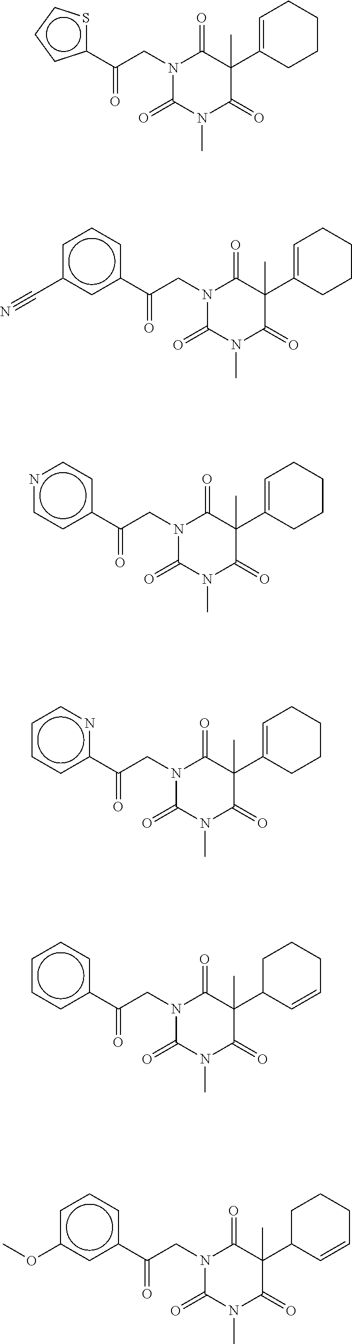

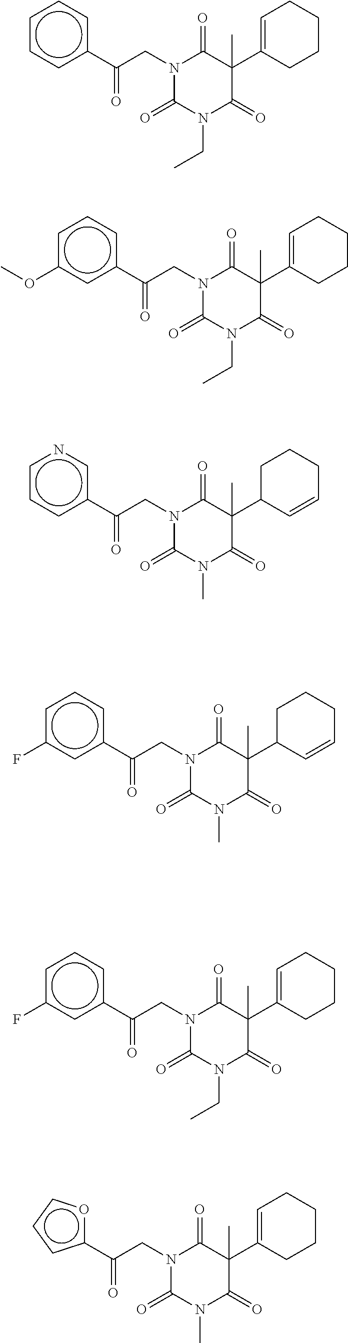

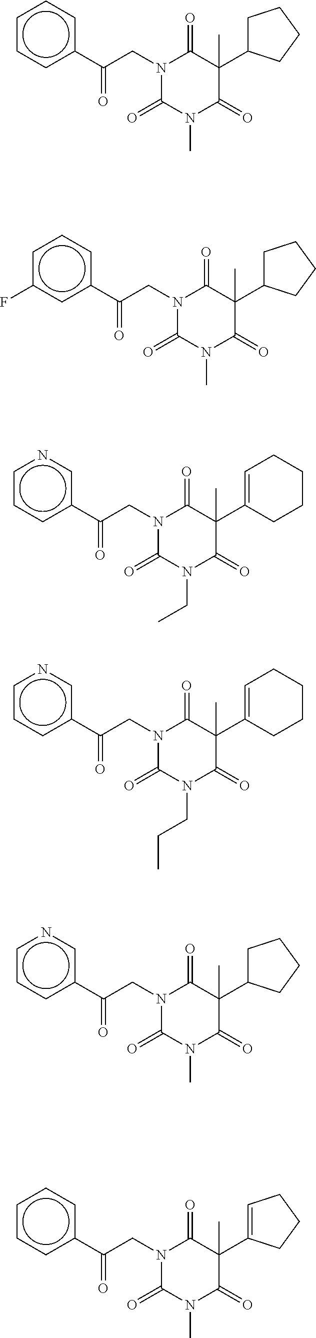

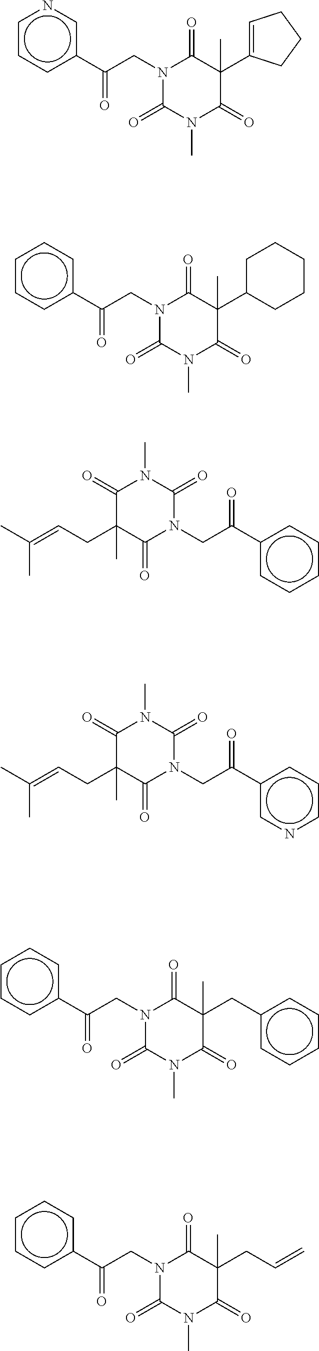

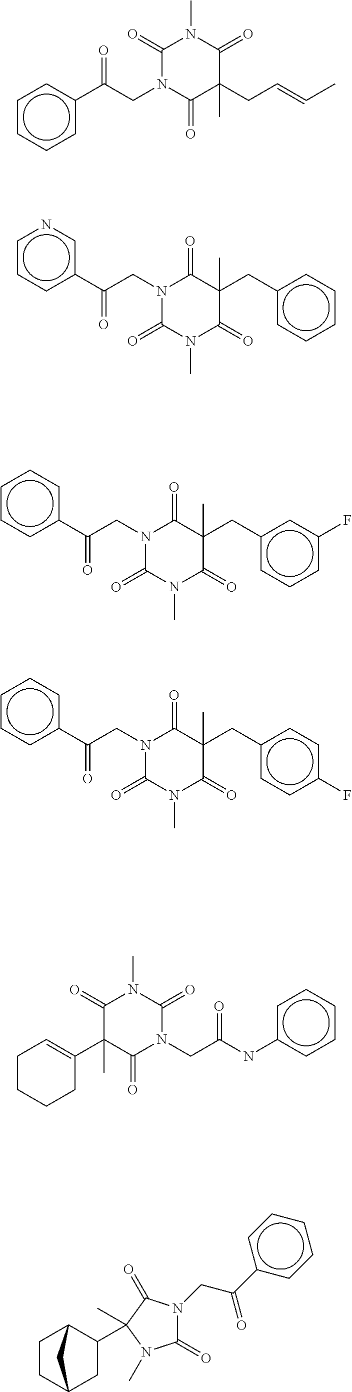

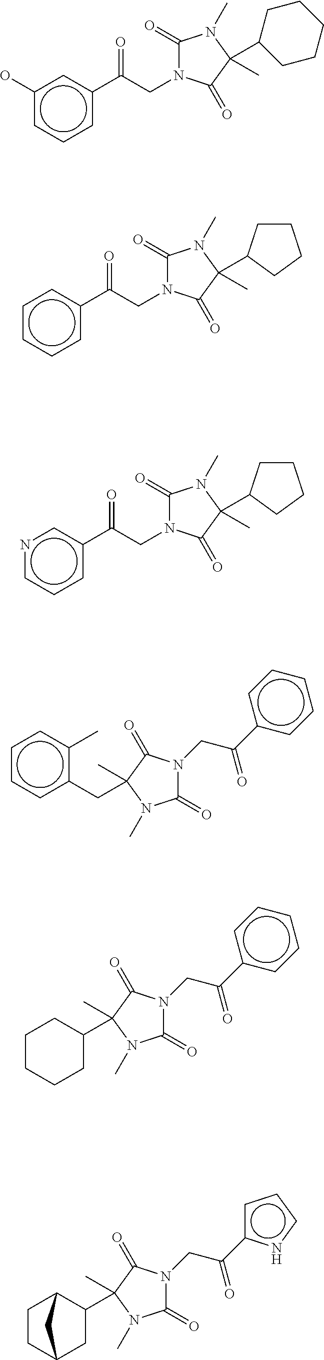

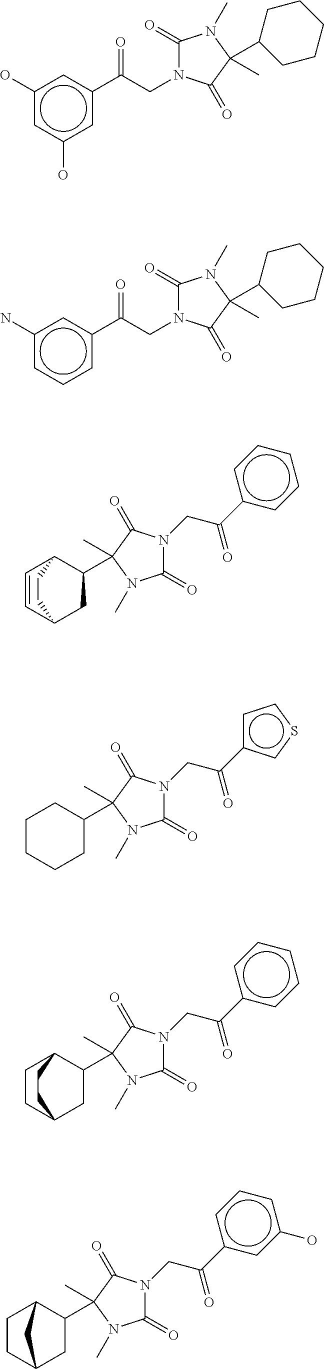

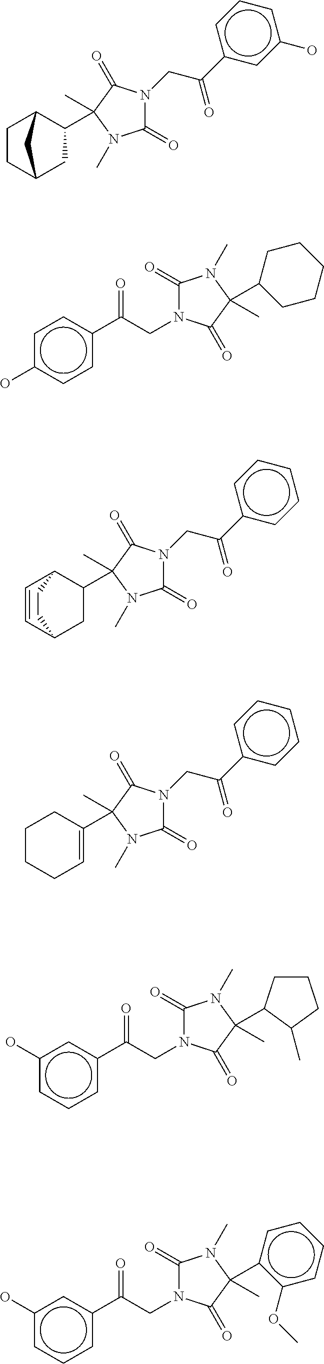

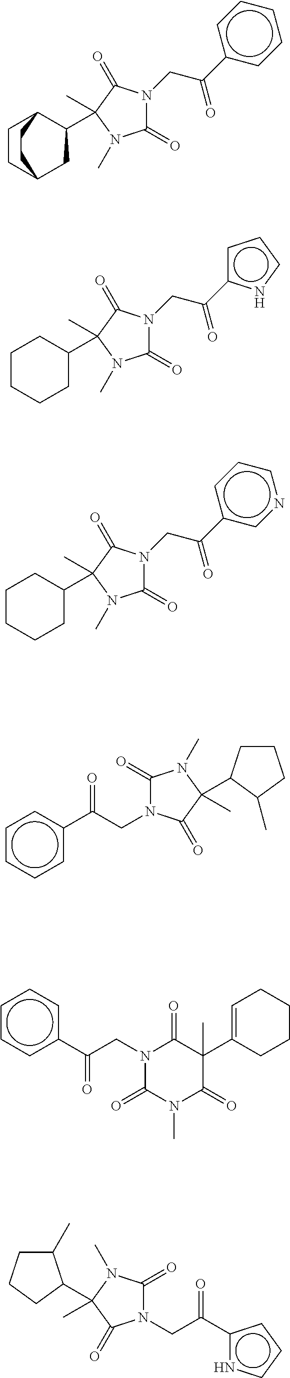

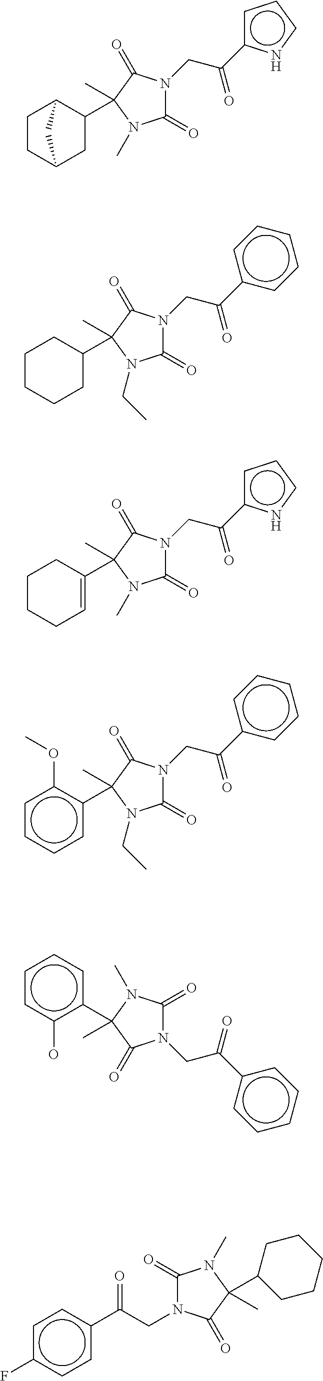

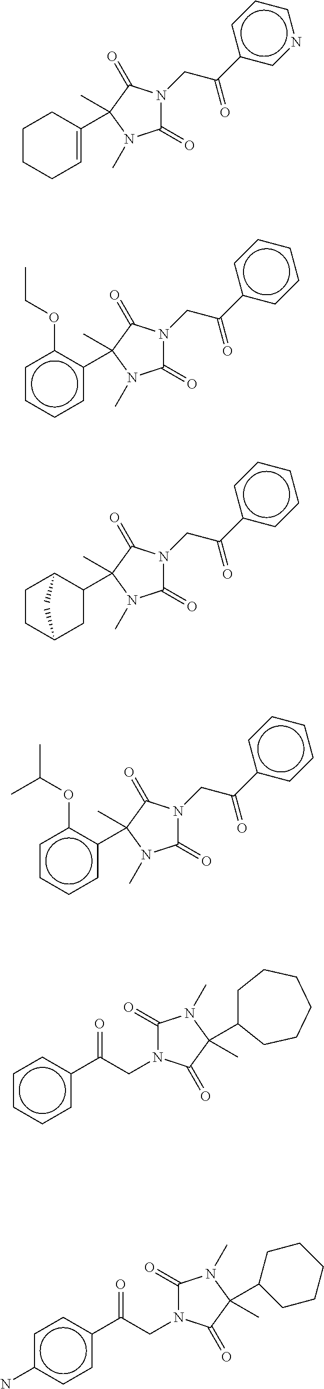

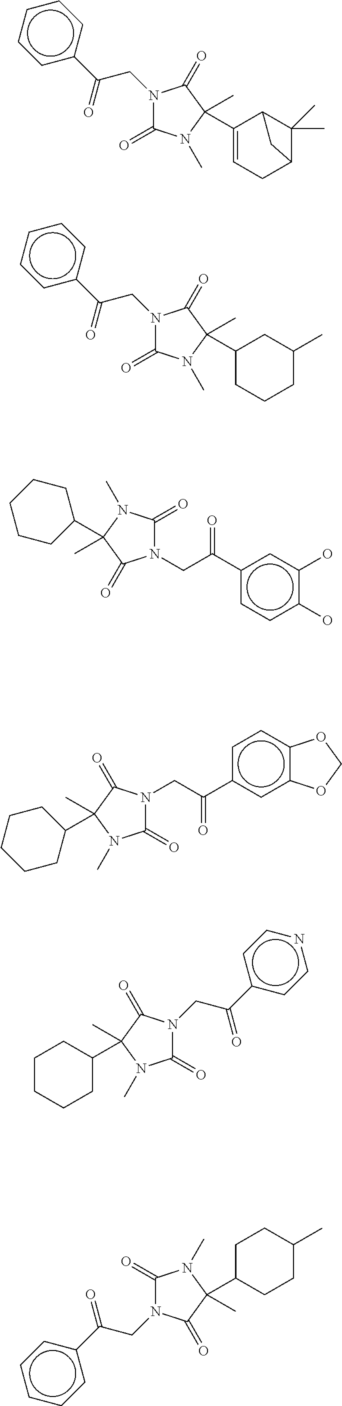

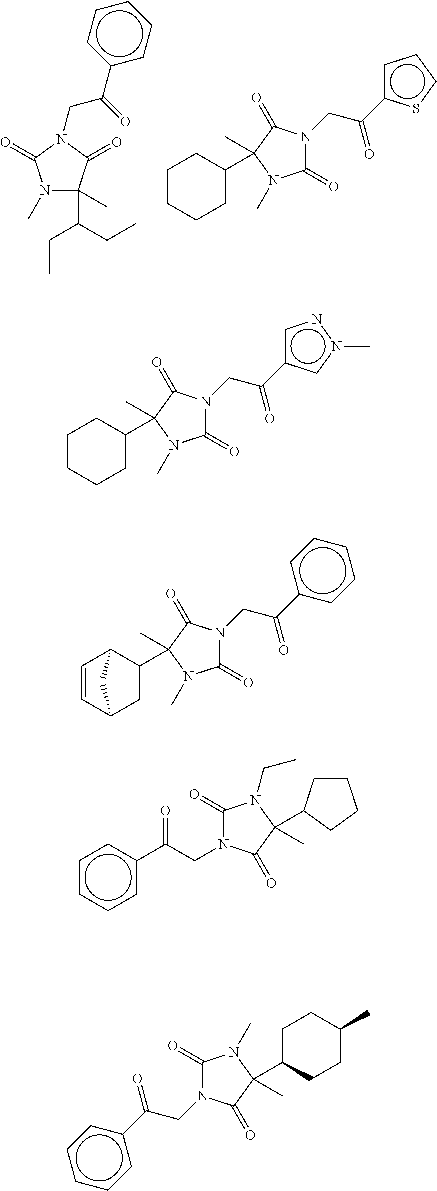

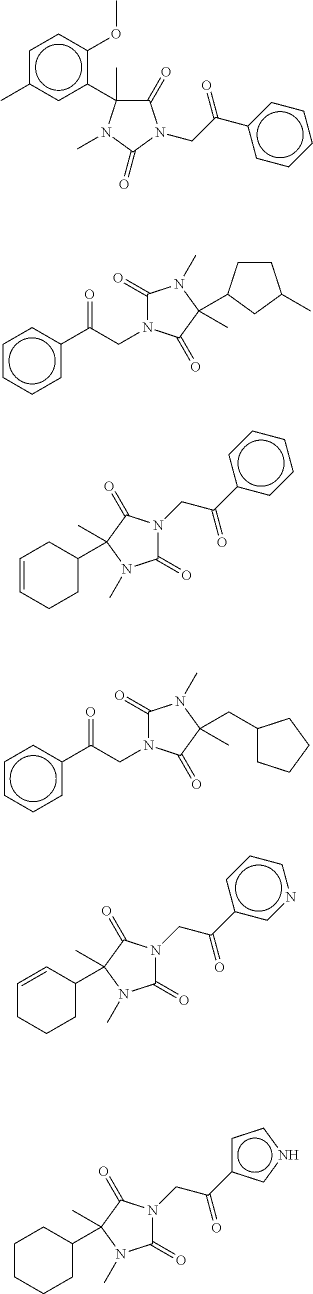

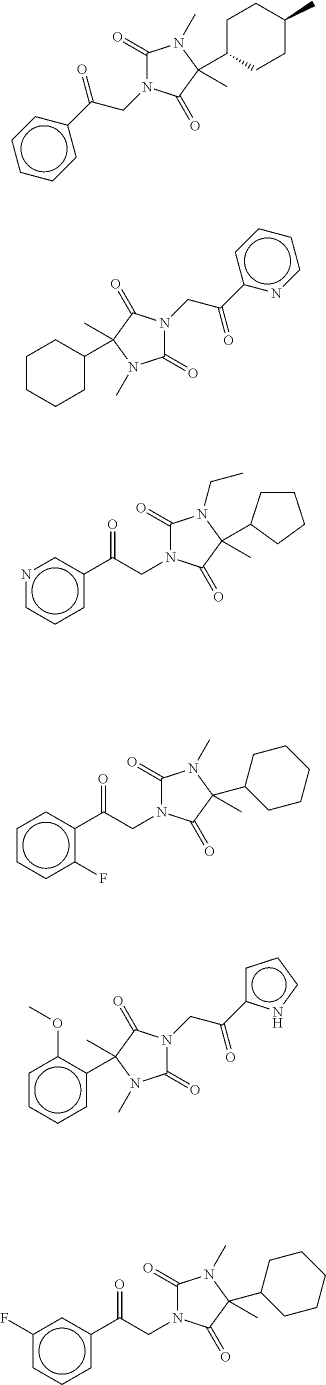

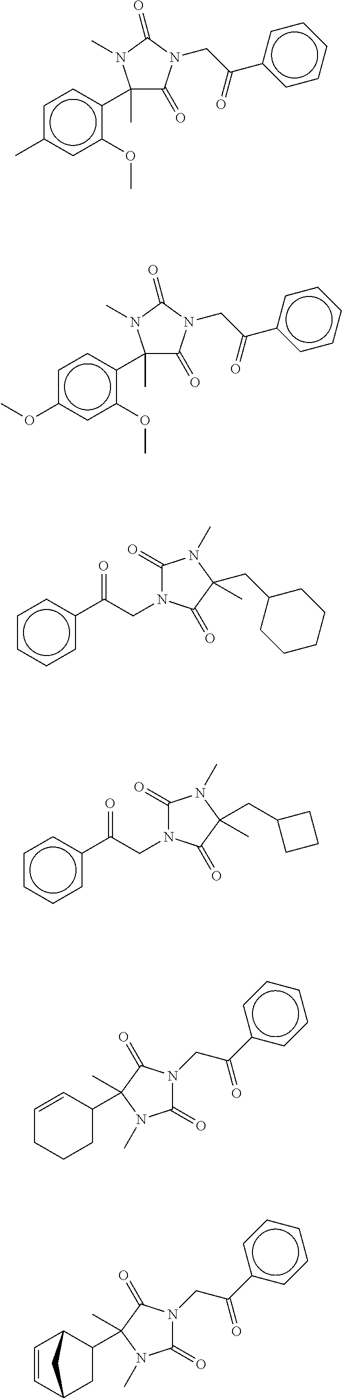

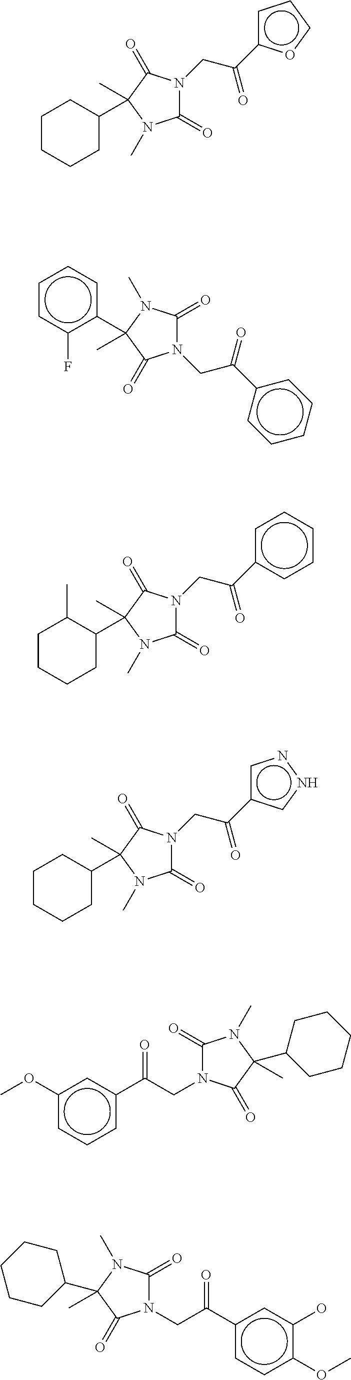

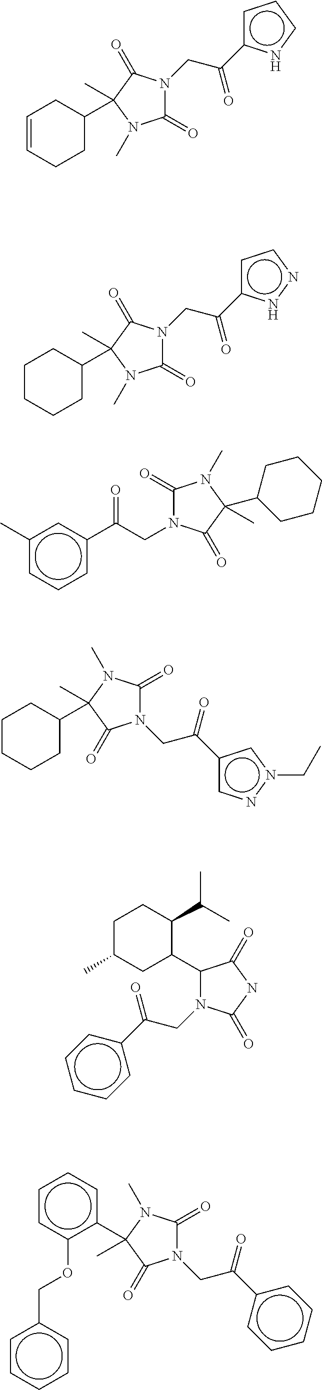

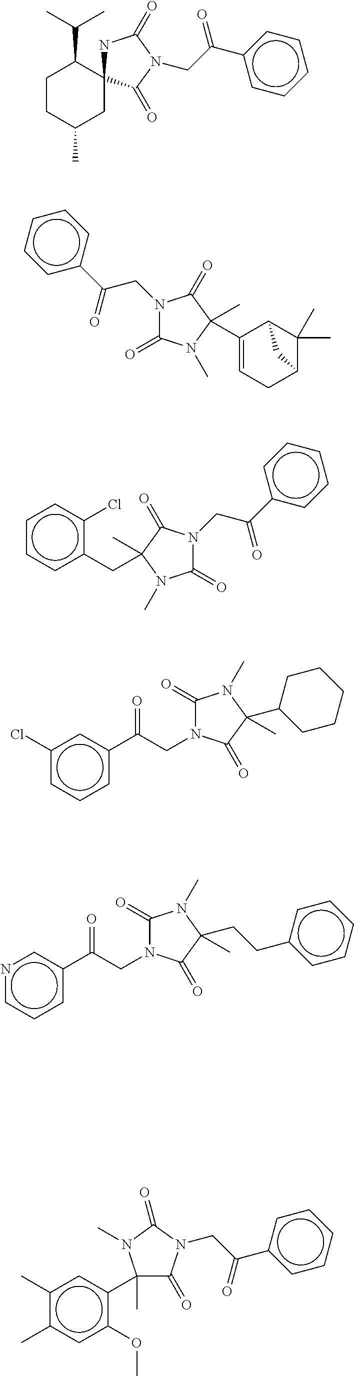

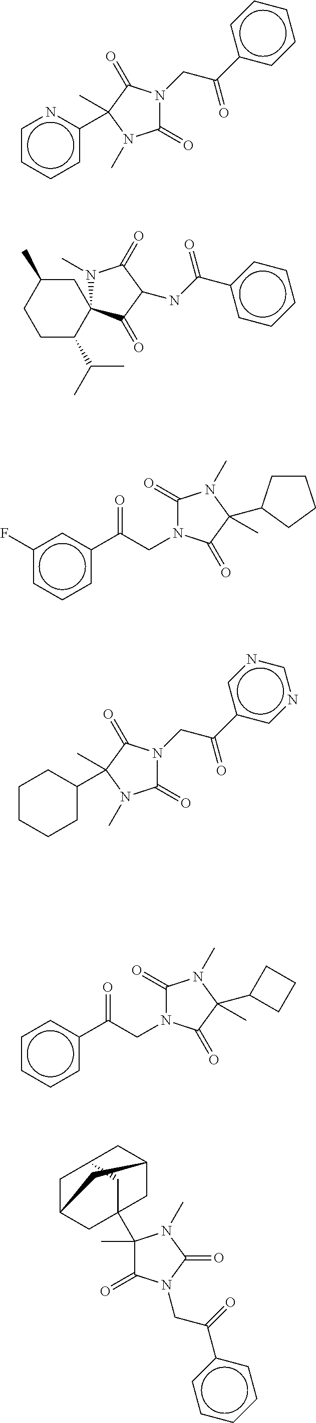

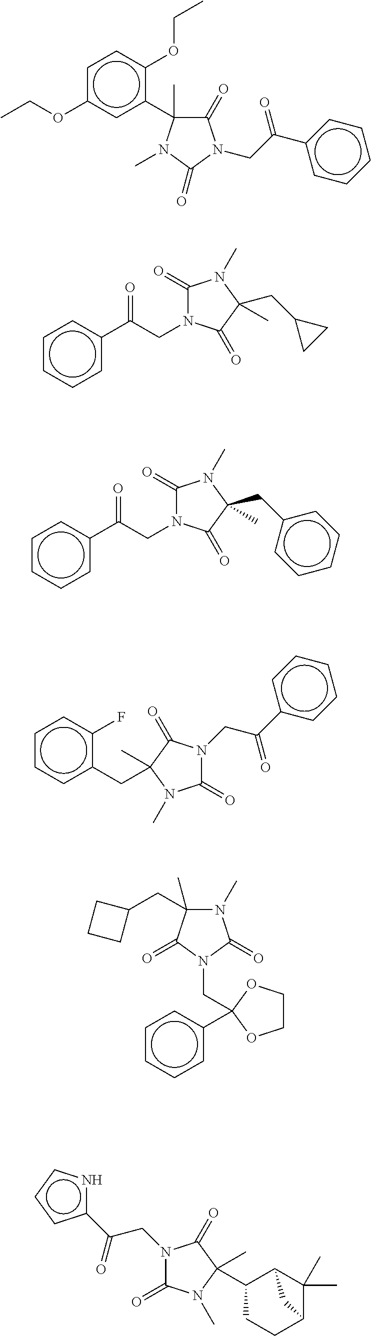

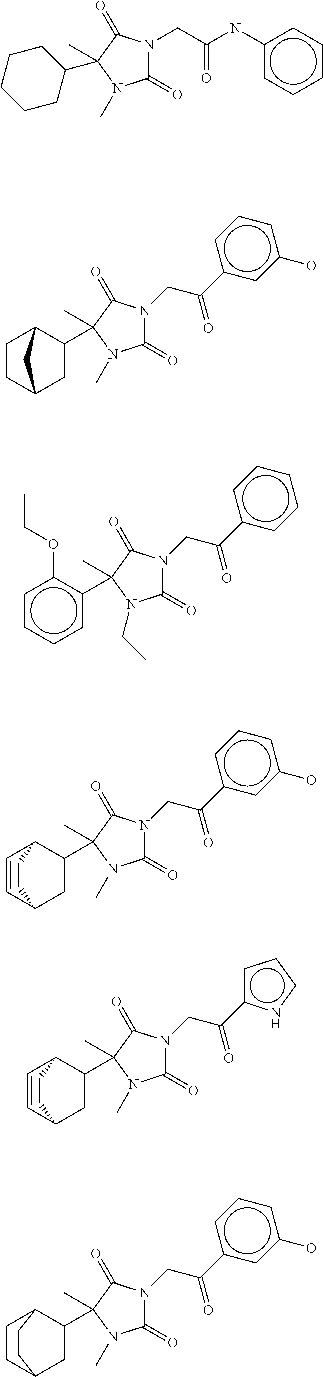

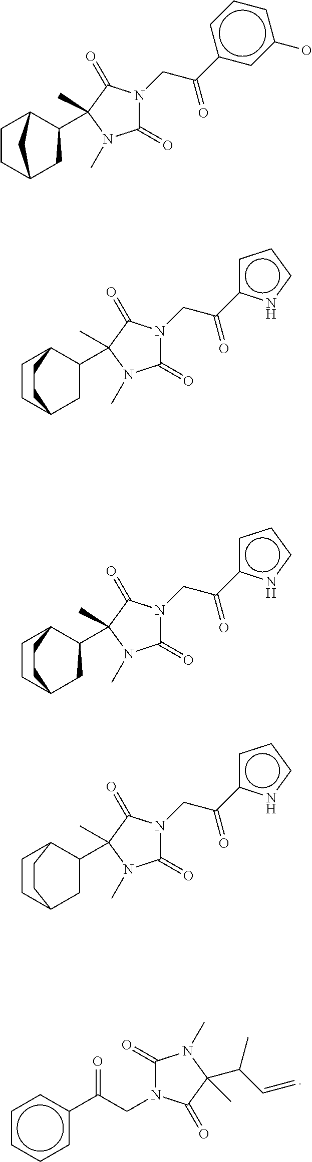

8. A topical composition comprising a carrier and a compound of Formula (III): 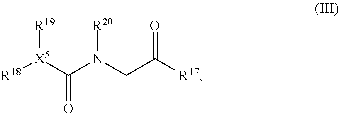
or a salt or solvate thereof;
wherein
X⁵ is CR²¹ or N;
R¹⁷ is optionally substituted aryl, optionally substituted carbocyclyl, optionally substituted heteroaryl, or optionally substituted heterocyclyl;
R¹⁸ and R¹⁹ are the same or different and are independently selected form the group consisting of hydrogen, optionally substituted alkyl, optionally substituted alkenyl, optionally substituted aryl, optionally substituted carbocyclyl, optionally substituted heteroaryl, and optionally substituted heterocyclyl;
R²⁰ is hydrogen, hydroxyl, alkoxy, optionally substituted alkyl, optionally substituted alkenyl, optionally substituted aryl, optionally substituted carbocyclyl, optionally substituted heteroaryl, or optionally substituted heterocyclyl; and
R²¹ is hydrogen, optionally substituted alkyl, optionally substituted alkenyl, optionally substituted aryl, optionally substituted carbocyclyl, optionally, substituted heteroaryl, or optionally substituted heterocyclyl; or alternatively, R¹⁹ and R²¹, or R¹⁹ and R²⁰, taken together with the atoms to which they are attached, form an optionally substituted carbocyclyl or optionally substituted heterocyclyl.

9. The composition of claim 7, wherein the compound of Formula (III) is selected from the group consisting of:

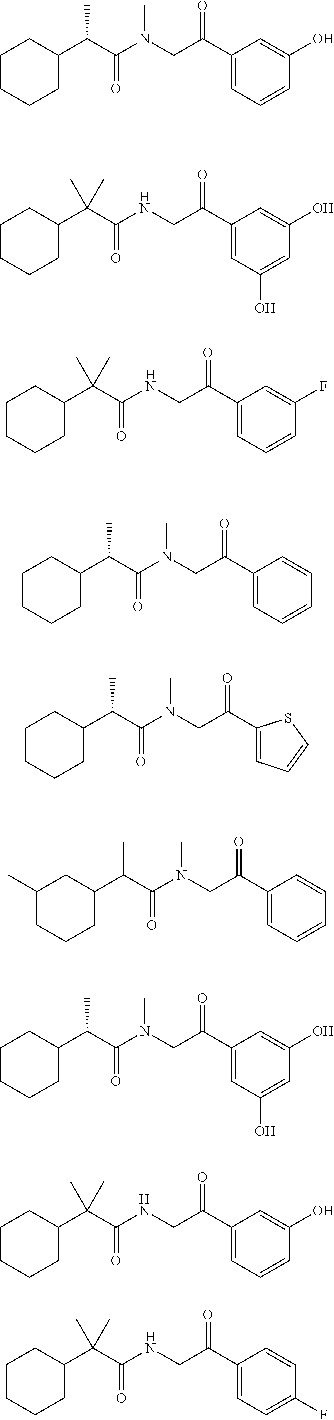

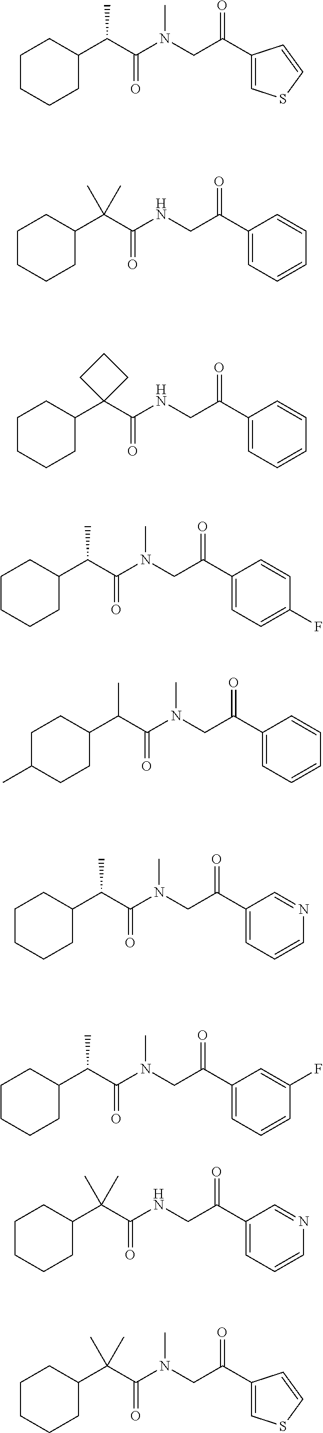

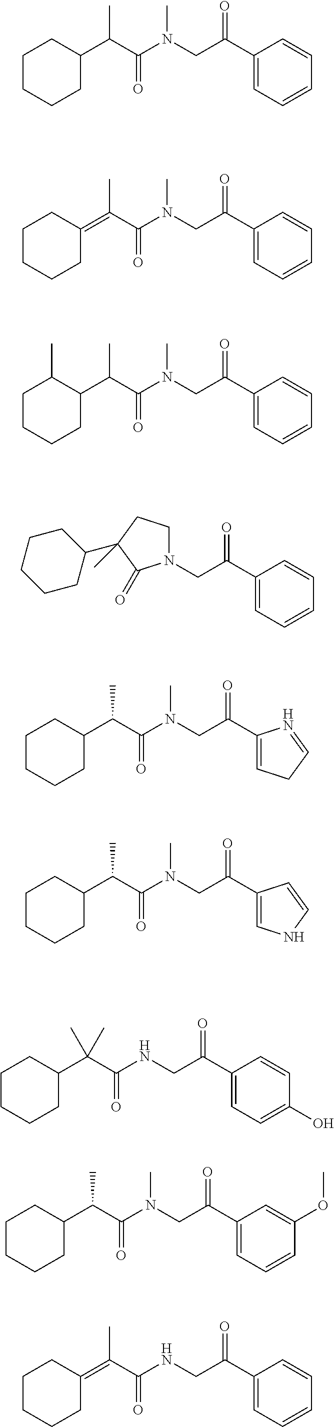

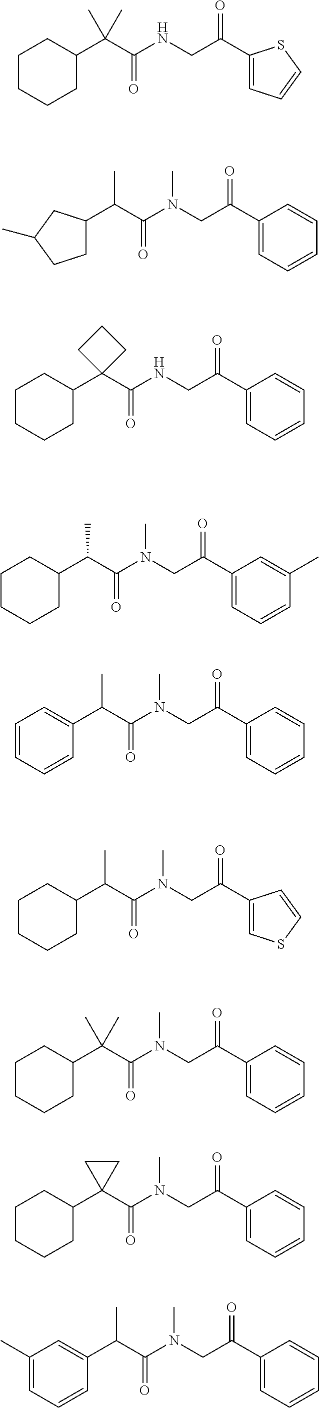

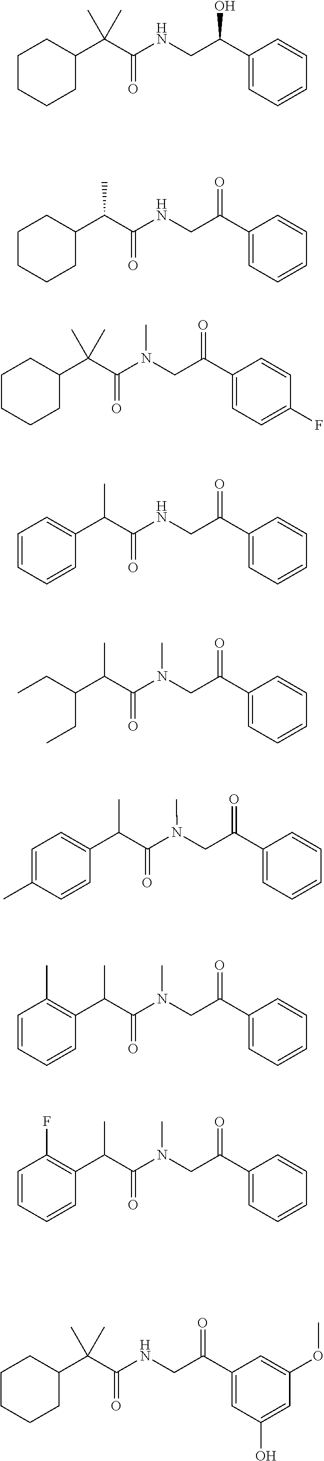

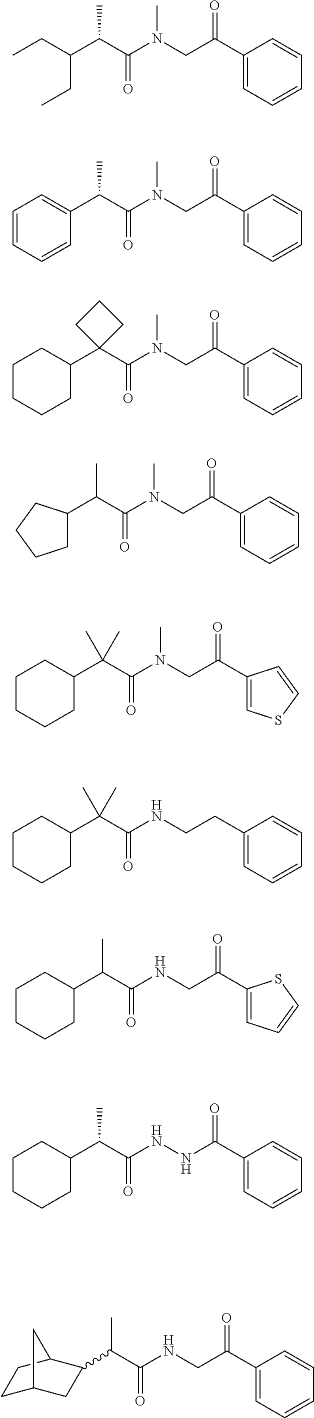

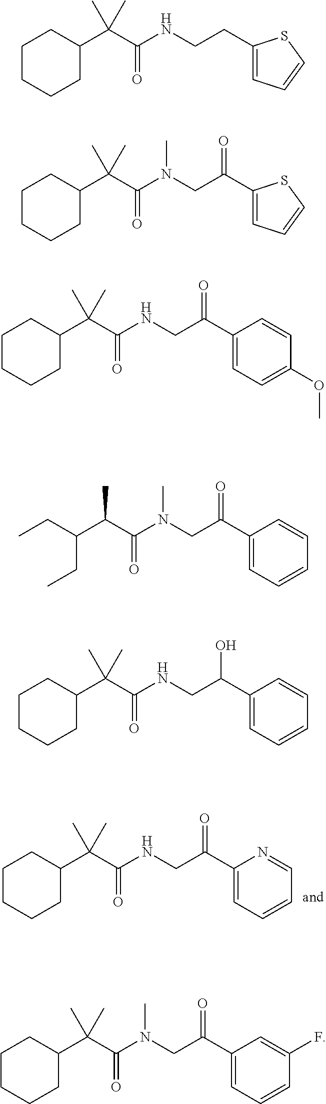

10. The composition of claim 1, wherein the composition is in the form of a solid, semi-solid, plaster, solution, suspension, lotion, cream, foam, gel, paste, poultice, emulsion, or a combination thereof.

11. The composition of claim 10, wherein the composition is selected from the group consisting of a poultice for the treatment of burns, an anti-itch cream, an antibiotic ointment, an after-sun gel, and an after-sun lotion.

12. A method of treating insect bites, insect stings, allergenic effects, burns, scrapes, cuts, abrasions, psoriasis, dandruff, pruritus, itching, nasal complaints, sore throats, upper respiratory ailments, acne, athlete's foot, or skin irritation as a result of contact with poison ivy, poison oak, or poison sumac comprising applying the composition of claim 1 to a subject in need thereof.

13. The composition of claim 1, wherein the composition is a personal care product selected from the group consisting of shaving products, deodorants, odorants, insect repellants, facial care products, body care products, cosmetics, soap products, and lip products.

14. The composition of claim 13, wherein the composition is selected from the group consisting of a shaving cream, a shaving lotion, an after-shave lotion, a roll-on deodorant, a spray deodorant, an air freshener, a room deodorizer, a perfume, a cologne, a hand-soap, a facial soap, a lipstick, a lip balm, a lip gloss, a body lotion, and a shower gel.

15. A method of enhancing massage therapy treatment comprising applying the composition of claim 1 to a subject in need thereof.

16. The composition of claim 1, wherein the composition is an aphrodisiac.
